(19) United States
(12) Patent Application Publication
(10) Pub. No.: US 2015/0367144 A1
(43) Pub. Date: Dec. 24, 2015

(54) ADVANCED ROTATING-SHIELD BRACHYTHERAPY AND PLANNING OF THE SAME

(71) Applicant: University of Iowa Research Foundation, Iowa City, IA (US)

(72) Inventors: Ryan Flynn, Iowa City, IA (US); Xiaodong Wu, Coralville, IA (US); Yunlong Liu, Iowa City, IA (US); Quentin Adams, Iowa City, IA (US); Drake Edwards, Iowa City, IA (US); Shirin Enger, Uppsala (SE); Brittany Rae Woodin, Milledgeville, IL (US)

(21) Appl. No.: 14/418,274

(22) PCT Filed: Mar. 15, 2013
(86) PCT No.: PCT/US13/32071

§ 371 Date: Jan. 29, 2015

Related U.S. Application Data
(60) Provisional application No. 61/678,082, filed on Jul. 31, 2012.
(60) Provisional application No. 61/678,080, filed on Jul. 31, 2012.
(60) Provisional application No. 61/740,086, filed on Dec. 20, 2012.

(51) Int. Cl. A61N 5/10

(52) U.S. Cl. CPC A61N 5/1031; A61N 5/1027; A61N 5/1007; A61N 5/1039; A61N 5/1048; A61N 2005/1094; A61N 2005/1008; A61N 2005/1035; A61N 2005/1074

(57) ABSTRACT

Systems and methods for rotating shield brachytherapy. In an aspect, some of the systems and methods can be used to facilitate shield selection for use in rotating shield brachytherapy. In an aspect, the invention is a shielded needle or catheter system with a rotational controller for delivering radioisotope-based interstitial rotating shield brachytherapy (I-RSBT), In an aspect, I-RSBT needles can deliver dose distributions that can be non-radially symmetric about each needle, enabling reduced doses to sensitive normal tissues. Further provided are methods and systems for selecting an emission angle for use in S-RSBT and for sequencing the rotating shields. Further provided are methods and system for the multiple application of M-RSBT in a single setting.

### CROSS-REFERENCE TO RELATED APPLICATIONS

This application claims priority from U.S. Provisional Patent Applications 61/678,080, filed on Jul. 31, 2012; 61/678,082, filed on Jul. 31, 2012; and 61/740,086 filed Dec. 20, 12012. The contents of each of the foregoing documents are incorporated herein by reference.

### BACKGROUND

High-dose-rate brachytherapy (HDR-BT) is a technique for treating cancerous tumors in which needles are inserted inside or close to the tumor. A radiation source travels inside each needle, depositing a radiation dose pattern inside the tumor over one or more treatment sessions, with the goal of killing all of the tumor cells and sparing radiation-sensitive normal tissue as much as possible.

### SUMMARY OF THE INVENTION

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Certain embodiments of the disclosure relate to methods for facilitating shield selection for use in single rotating shield brachytherapy. Such methods decouple the sequencing procedure from the dose optimization resulting in a reduction in treatment planning time as well as allow treatment providers to quickly select the optimal emission angle for a clinical case given the prescribed treatment time and dose. Certain other embodiments of the disclosure relate to methods for facilitating a method for sequencing the rotating shields in dynamic rotational shield brachytherapy. Such methods improve dose conformity without compromising adjacent healthy tissue within an acceptable delivery time relative to conventional techniques.

The invention is a shielded needle or catheter system with a rotational controller for delivering radioisotope-based interstitial rotating shield brachytherapy (I-RSBT). The I-RSBT system is applicable in the fields of radiation oncology and urology. The I-RSBT system overcomes the primary limitation of conventional interstitial HDR-BT, which is that individual needles can only deliver dose distributions that are radially symmetric about each needle. With I-RSBT, the shielded needles deliver dose distributions that are deliberately non-radially symmetric about each needle, enabling reduced doses to sensitive normal tissues. For prostate cancer patients, for example, a dose reduction to normal tissues enables reduced urethral (incontinence, urethral stricture) and rectal (bowel dysfunction) complications relative to conventional BT. In addition, reduced normal tissue doses could enable increased doses to the prostate cancer, potentially reducing the number of treatment sessions needed to deliver the therapy, which is typically two to four. In one aspect, I-RSBT is of significant commercial value because it could be the least expensive, lowest-complication-rate therapy for the nearly 180,000 patients who are diagnosed with localized prostate cancer per year in the U.S.

Embodiments of the invention can comprise an apparatus and method for modulating the intensity of x-rays or gamma-rays from a radiation source used to treat cancerous tumors, called multiple rotating shield brachytherapy (M-RSBT) and is applicable in the field of radiation oncology. Conventional brachytherapy (BT) entails the insertion of radioactive sources into tumors through interstitial needles or intracavitary applicators, and delivers very high radiation doses to tumors but often with poor tumor dose conformity, as conventional BT dose distributions are radially symmetric and tumors are usually not. This is of concern since tumor underdosage can lead to recurrence and tumor overdosage can damage nearby healthy tissue. Single rotating-shield brachytherapy (S-RSBT) uses a shielded BT source that emits more radiation at conventionally underdosed tumor regions and less radiation at conventionally overdosed tumor regions. However, the time necessary to treat a tumor with S-RSBT is inversely proportional to the shield emission angle, thus small emission angle shields produce long delivery times. M-RSBT significantly reduces intensity modulated brachytherapy (IMBT) treatment time by using intelligent combinations of varying emission angle shields. This invention is an apparatus that enables the fast, remote-controlled changing of radiation shields, and a method for rapidly determining which combination of radiation shields should be used for a given patient. The selection of shields is computer-optimized by specified source positions, tumor shape, and a desired dose distribution. The shields are composed of a high-density material such as tungsten, lead, gold, silver, or bismuth. M-RSBT is of commercial value because it is a method that provides a significant improvement over conventional BT and S-RSBT methods. Examples of cancers that we believe can be treated more effectively with M-RSBT include cervical, vaginal, endometrial, colorectal, prostate, and breast cancers.

Further provided are methods and systems for selecting an emission angle for use in S-RSBT. An example method can comprise calculating a dose, optimizing the calculated dose, generating a first treatment plan based on the optimized dose, generating a second treatment plan, and selecting one of the first treatment plan or the second treatment plan.

Further provided are methods and systems for sequencing the rotating shields. An exemplary method can comprise calculating a dose, optimizing the dose, and generating a treatment plan based on an optimal sequence of the dose. Further provided are methods and system for the multiple application of M-RSBT in a single setting.

Additional aspects, features, or advantages of the subject disclosure will be set forth in part in the description which follows, and in part will be obvious from the description, or may be learned by practice of the subject disclosure. The advantages of the subject disclosure will be realized and attained by means of the elements and combinations particularly pointed out in the appended claims. It is to be understood that both the foregoing general description and the following detailed description are exemplary and explanatory only and are not restrictive of the subject disclosure.

### BRIEF DESCRIPTION OF THE DRAWINGS AND APPENDICES

The accompanying drawings are incorporated and illustrate exemplary embodiments of the disclosure and together with the description and claims appended hereto serve to explain various principles, features, or aspects of the subject disclosure.

FIG. 1A illustrates a cross-sectional view of an exemplary partially shielded BT source in RSBT. FIG. 1B illustrates a longitudinal-sectional view of the beamlets arrangement.

FIG. 2 illustrates a three dimensional view of an exemplary clinical cervical cancer case.

FIG. 3 is a flowchart showing the treatment planning steps of an exemplary method for treatment plan selection using exhaustive replanning (either simulated or gradient-based) and using optimal sequencing with anchor plans.

FIG. 4 illustrates exemplary dose distributions of different RSBT treatment plans generated for Patient 1 on MRI scan slice 30.

FIG. 5 illustrates exemplary dose distributions of different RSBT treatment plans generated for Patient 2 on MRI scan slice 30.

FIG. 6 is a table of an exemplary quantitative comparison between the dose quality of different delivery plans for Patient 1.

FIG. 7 is a table of an exemplary quantitative comparison between the dose quality of different delivery plans for Patient 2.

FIG. 8 is an exemplary Pareto plot generated using rapid emission angle selection (REAS) for Patient 1.

FIG. 9 is an exemplary Pareto plot generated using REAS for Patient 2.

FIGS. 10(a-b) are schematic illustrations of emission angles according to an aspect.

FIGS. 11(a-d) illustrate a sequencing optimization model according to an aspect.

FIG. 12 illustrates an MRI 2D slice of a cervical cancer case used for verification in an aspect.

FIG. 13 is a table that compares plan quality and delivery times of the S-RSBT sequencing algorithm under different fan-angle and the conventional isotropic one according to an aspect. The prescription dose is normalized to 100, the volumes are also measured in percentage, and the delivery times are also listed with a relative time unit.

FIG. 14 is a table that compares plan quality and delivery times of the S-RSBT sequencing algorithm under different fan-angle with the maximum allowed combination of unshielded source and the conventional isotropic one. The prescription dose is normalized to 100, the volumes are also measured in percentage, and the delivery times are also listed with a relative time unit.

FIG. 15 shows a comparison of Dose-Volume Histogram (DVH) plots for IMBT with different settings and conventional BT.

FIGS. 16(a)-16(b) show graphs of delivery time vs. D90 for every possible azimuthal emission angle and time bound for two exemplary clinical cases.

FIG. 17 illustrates exemplary dose distributions for selective delivery configurations for two exemplary clinical cases.

FIG. 18 is a table showing dose quality and delivery time comparison between different delivery configurations for two patients according to an aspect.

FIG. 19 illustrates a circular integral block decomposition (CIBD) problem according to an aspect.

FIG. 20 illustrates a cross-sectional view of an exemplary rotationally shielded source in D-RSBT according to an aspect.

FIG. 21 is an exemplary optimal sequencing (OSD) generated Pareto plot.

FIG. 22 is a table comparing different delivery methods with OSD.

FIG. 23 is a flowchart showing the treatment planning steps of an exemplary method for treatment plan selection using optimal sequencing methods for D-RSBT.

FIG. 24 shows a comparison of DVH plots for baseline anchor plans and selected OSD generated plans.

FIG. 25 is a table of an exemplary quantitative comparison between the dose quality between the baseline anchor plans and selected OSD generated plans.

FIG. 26 illustrates mapping from ( [symbol] , [symbol] ) to ( [symbol] , [symbol] ).

FIG. 27(a) illustrates the impact parameter n can have on the running time in the five exemplary clinical cases used. FIG. 27(b) illustrates the impact parameter H can have on the running time in the five exemplary clinical cases used.

FIG. 28 is a table showing the plan quality comparisons for five exemplary clinical cases generated using the disclosed methods for optimizing treatment delivery of D-RSBT.

FIG. 29 illustrates one example of a DVH plot for one of the five exemplary clinical cases generated using the disclosed methods for optimizing treatment delivery of D-RSBT.

FIG. 30 illustrates a comparison of the results obtained by conventional BT versus using the disclosed methods for optimizing treatment delivery of D-RSBT for another exemplary clinical case.

FIG. 31(a) shows one example of a dose distribution for conventional ¹⁹²Ir unshielded source for an exemplary clinical case. FIG. 31(b) shows a dose distribution for IMBT using the Axxent Xoft eBT source configured with 60 divisions and no overlapping applied to the same exemplary clinical case. FIG. 31(c) shows a dose distribution for IMBT using the Axxent Xoft eBT source as well as the disclosed methods for optimizing treatment delivery of D-RSBT applied to the dame exemplary clinical case.

FIG. 32 illustrates a computing environment that enables various aspects of treatment planning and/or automation of treatment planning in accordance with aspects described herein.

FIG. 33 illustrates example interstitial brachytherapy (BT) for localized prostate cancer.

FIG. 34 illustrates radii of candidate source/shield combinations for RSBT, showing the different source sizes that would be required to produce the same dose rate in water at 1 cm lateral to the source axis as a 10 Curie ¹⁹²Ir source.

FIG. 35 illustrates an example embodiment of a shielded catheter for I-RSBT delivery with a sharp tip for use as a needle in accordance with one or more aspects of the disclosure. Blunt-tipped versions are also possible, which can be used as catheters.

FIGS. 36A-F illustrate examples of I-RSBT catheter cross sections having a centered lumen and an off-center lumen according to aspects of the present invention

FIGS. 37A-37B: Example prototypes of (a) 17 gauge stainless steel I-RSBT catheter with a 180° platinum shield and a plastic docking device (left). The grasping device mounts to a translating rotational motor and connects to each needle, enabling the delivery of 1-RSBT plans in series, (b) needle grasping device and connector to permit rotation.

FIG. 38A illustrates examples of simulated localized prostate cancer treatments with unshielded BT and I-RSBT. The top row: dose-volume histograms (DVHs). Second row: conventional unshielded interstitial BT dose distributions. Bottom row: I-RSBT dose distributions. FIG. 38B illustrates another example of simulated localized prostate cancer treatments with unshielded BT and I-RSBT according to an aspect.

FIGS. 39A-39C illustrate example embodiments of docking devices in accordance with one or more aspects of the disclosure.

FIG. 40: (a) 3-D rendering of cervical cancer treatment geometry for T&O delivery. (b) The conventional BT dose distribution, underdoses the clinical target volume (CTV). (c) The IMBT dose distribution enhances CTV coverage and spares bladder/rectum.

FIGS. 41A-C are schematic representations of dwell times for a 90° emission angle with four locations using S-RBST and M-RSBT according to an aspect.

FIGS. 42A-42D illustrate example cross sections of example intensity modulated brachytherapy (IMBT) insertion devices, which could be an intracavitary applicator of inner radius r_{ID} and outer radius rₜₒₜ.

FIG. 43 illustrates an additional or alternative example cross section of the RS-IMBT delivery system.

FIG. 44 illustrates an example process that permits an M-RSBT apparatus to change radiation shields in order to deliver M-RSBT in accordance with one or more aspects of the disclosure.

FIG. 45 presents definitions of shield emission angle, direction, and beamlet combination in accordance with one or more aspects of the subject disclosure.

FIGS. 46(a-b) present the dose distribution for a patient that would benefit less from M-RSBT using a minimum emission angle of 180° and the analogous distribution using a minimum emission angle of 22.5°.

FIG. 47 presents a comparison of M-RSBT treatment times (green) and RSBT treatment times (blue) plotted against the D₉₀ for a tumor surface in accordance with one or more aspects of the disclosure.

FIG. 48: (a) The dose distribution for a patient that would benefit significantly from M-RSBT using a minimum emission angle of 180°. (b) The analogous distribution using a minimum emission angle of 22.5°.

FIG. 49 presents a comparison of M-RSBT treatment times (green) and RSBT treatment times (blue) plotted against the D₉₀ for a tumor surface in accordance with one or more aspects of the disclosure.

FIG. 50 illustrates an example comparison of treatment times for RSBT and M-RSBT (also referred to as MRS-IMBT) in accordance with one or more aspects of the disclosure.

FIG. 51 illustrates an example computing environment that permits implementation of therapy design in accordance with one or more aspects of the disclosure.

FIGS. 52(a)-(b) illustrate an exemplary capsule, radiation source and wire.

FIG. 53 is a flowchart illustrating an exemplary method of forming a therapeutic radiation capsule.

FIG. 54 is a flowchart illustrating an exemplary method of providing therapeutic radiation.

FIG. 55 is a flowchart illustrating an exemplary method for rotating shield brachytherapy (RSBT).

FIG. 56 is a flowchart illustrating an exemplary method for selecting an emission angle for use in single rotating-shield brachytherapy.

FIG. 57 is a flowchart illustrating an exemplary method for sequencing rotating shields.

FIG. 58 is a schematic illustration of a RSBT application system according to an aspect.

FIGS. 59-60 are perspective see-through views of a catheter control cartridge of the RSBT application system according to an aspect.

FIG. 61 is a cross sectional view of at the distal end of a RSBT catheter of the RSBT application system according to an aspect.

FIG. 62 is a partial bottom perspective view of a portion of the catheter control cartridge of FIGS. 59-60.

FIG. 63 is a top perspective view of a cartridge magazine of the RSBT application system according to an aspect.

FIG. 64 is a back plane view of the proximal end of cartridge magazine of FIG. 63, seeing through the interior of the distal end.

FIG. 65 is a front plane view of a mount of the RSBT application system according to an aspect.

FIG. 66a-66d are schematic representations of a mount of the RSBT application system according to an aspect.

FIG. 67 is a front plan view of a shelf and a cartridge of a RSBT application system according to an aspect.

FIG. 68 is a partial top perspective see-through view of components of a RSBT application system according to an aspect.

FIG. 69 is a schematic illustration of the orientation for needles within a subject according to an aspect.

FIGS. 70-71 are schematic illustrations of the use of the RSBT application system according to an aspect.

Appendices A-B described various exemplary features of M-RSBT and I-RSBT in accordance with one or more aspects of the disclosure.

Appendix C shows additional information about optimal sequencing in rotating shield intensity modulated brachytherapy.

Appendix D shows additional information about optimal emission angle selection in rotating shield brachytherapy

Appendix E shows additional information about intensity modulated brachytherapy.

Appendix F shows additional information about dynamic rotating shield intensity modulated brachytherapy.

Appendix G shows additional information about multiple rotating shield brachytherapy.

Appendix H shows additional information about interstitial rotating shield brachytherapy.

Appendix I provides terminology pertinent to the disclosure and bibliographic information.

### DETAILED DESCRIPTION

The subject disclosure may be understood more readily by reference to the following detailed description of exemplary embodiments of the subject disclosure and to the Figures and their previous and following description.

Before the present compounds, compositions, articles, devices, and/or methods are disclosed and described, it is to be understood that the subject disclosure is not limited to specific systems and methods for shield-based brachytherapy and related devices. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In the subject specification and in the claims which follow, reference may be made to a number of terms which shall be defined to have the following meanings: “optional” or “optionally” means that the subsequently described event or circumstance may or may not occur, and that the description includes instances where said event or circumstance occurs and instances where it does not.

As employed in this specification and annexed drawings, the terms “unit,” “component,” “interface,” “system,” “platform,” “stage,” and the like are intended to include a computer-related entity or an entity related to an operational apparatus with one or more specific functionalities, wherein the computer-related entity or the entity related to the operational apparatus can be either hardware, a combination of hardware and software, software, or software in execution. One or more of such entities are also referred to as “functional elements.” As an example, a unit may be, but is not limited to being, a process running on a processor, a processor, an object, an executable computer program, a thread of execution, a program, a memory (e.g., a hard disc drive), and/or a computer. As another example, a unit can be an apparatus with specific functionality provided by mechanical parts operated by electric or electronic circuitry which is operated by a software or a firmware application executed by a processor, wherein the processor can be internal or external to the apparatus and executes at least a part of the software or firmware application. In addition or in the alternative, a unit can provide specific functionality based on physical structure or specific arrangement of hardware elements. As yet another example, a unit can be an apparatus that provides specific functionality through electronic functional elements without mechanical parts, the electronic functional elements can include a processor therein to execute software or firmware that provides at least in part the functionality of the electronic functional elements. An illustration of such apparatus can be control circuitry, such as a programmable logic controller. The foregoing example and related illustrations are but a few examples and are not intended to be limiting. Moreover, while such illustrations are presented for a unit, the foregoing examples also apply to a component, a system, a platform, and the like. It is noted that in certain embodiments, or in connection with certain aspects or features thereof, the terms “unit,” “component,” “system,” “interface,” “platform” can be utilized interchangeably.

Throughout the description and claims of this specification, the words “comprise,” “include,” and “have” and variations of the word, such as “comprising,” “comprises,” “including,” “includes,” “has,” and “having” mean “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Reference will now be made in detail to the various embodiment(s), aspects, and features of the subject disclosure, example(s) of which are illustrated in the accompanying drawings. Wherever possible, the same reference numbers are used throughout the drawings to refer to the same or like parts.

As described in greater detail below, this disclosure relates to methods for optimization of intensity modulated brachytherapy (IMBT). This disclosure relates, in one aspect, to a method for selecting the emission angle for use in single rotating-shield brachytherapy (S-RSBT) called rapid emission angle selection (REAS). In one aspect, REAS can enable a significant reduction in treatment time necessary to deliver an RSBT treatment by decoupling the planning and delivery of that treatment. In another aspect, decoupling the treatment planning and delivery can enable the treatment provider to quickly select a treatment plan that balances the delivery time and dose quality in RSBT based on the time budget for treatment and designated goal of the treatment plan. In yet another aspect, this disclosure relates to a method for sequencing the rotating shields with dynamic-sized opening in IMBT. In another aspect, this disclosure relates to a method for optimizing treatment delivery by approximating the dose distribution to the prescription within a given delivery time constraint.

Various aspects or features of the disclosure can be applied to the field of radiation oncology. Conventional brachytherapy entails the insertion of radioactive sources into tumors through interstitial needles or intracavitary applicators, and delivers very high radiation doses to tumors but often with poor tumor dose conformity. Without wishing to be bound by theory and/or simulation, such poor tumor dose conformity is due to the fact that conventional BT dose distributions typically are radially symmetric and tumors usually are not. It should be appreciated that poor dose conformity is of clinical concern since tumor underdosage leads to recurrence and tumor overdosage excessively damages nearby healthy tissue. One or more embodiments of the disclosure can rectify such deficiency by optimizing treatment delivery by at least one of approximating the dose distribution to the prescription within a given delivery time constraint and reducing the planning time cost while maintaining an acceptable approximation of the dose-volume optimization.

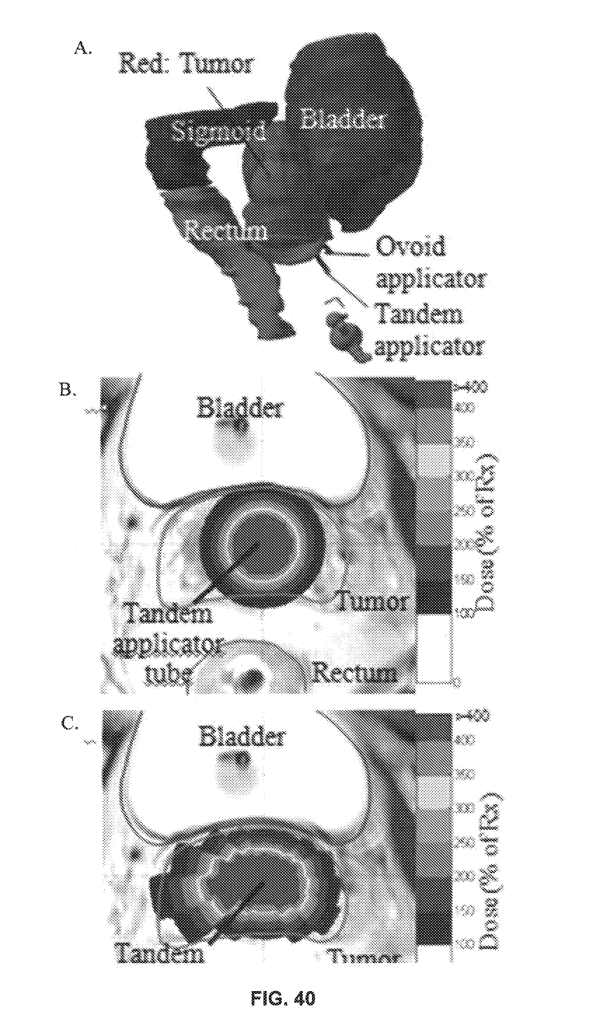

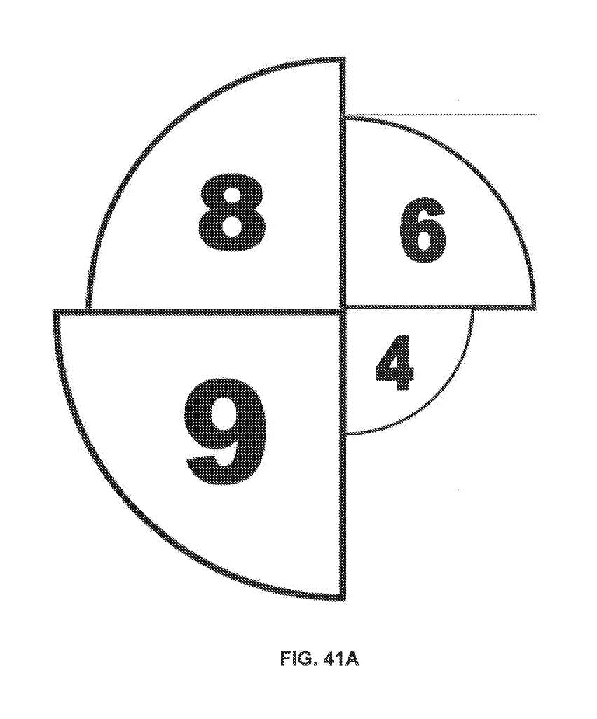

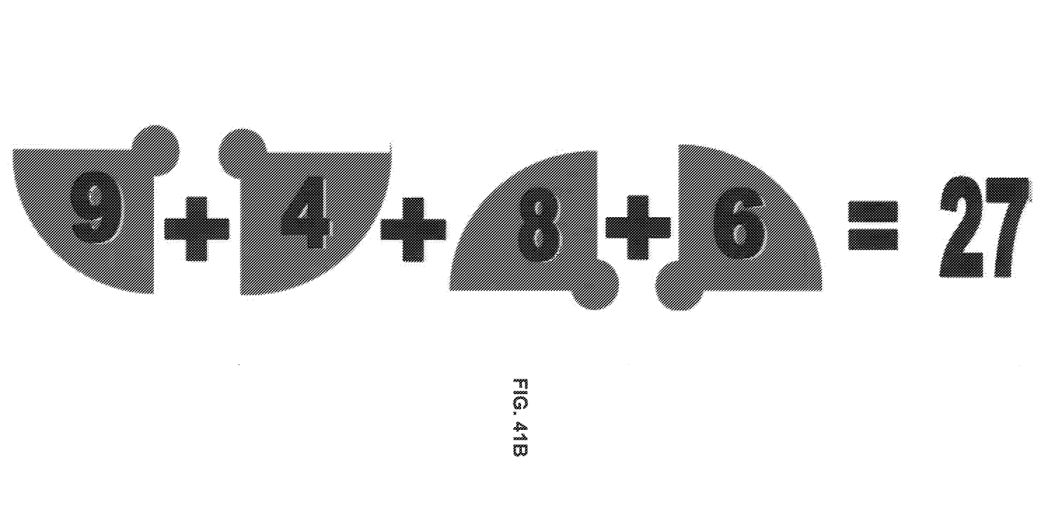

Brachytherapy, or “short-distance therapy,” treats target tissues, such as cancerous tumors, with radiation sources that can be placed inside or directly adjacent to the target tissue using some applicator. Example target tissues include cervical, vaginal, endometrial, breast, and skin cancers. Brachytherapy can be delivered with both interstitial and intracavitary applicators The advantage of brachytherapy over external beam radiation therapy (EBRT) is that EBRT beams usually must pass through healthy tissue in order to reach their targets, while the radiation used in brachytherapy may not. As a result brachytherapy can be used to treat targets with very high radiation doses relative to those achievable with EBRT, with less concern for overdosing nearby healthy tissue. The application of 3-D imaging systems such as USI, CT, and MRI for brachytherapy guidance has revealed that the dose conformity to tumors is often poor. Without wishing to be bound by theory and/or simulation, it is believed that poor conformity of conventional brachytherapy (BT) typically is delivered with isotopes or electronic sources that emit radiation in a radially symmetric manner, yet tumors often are not radially symmetric. For example, FIG. 2 illustrates MRI-generated 3D renderings of the anatomy of a patient being treated for cervical cancer, including the tumor and nearby critical structures: bladder, rectum, and sigmoid colon. The radiation is delivered with an X-ray or gamma-ray emitting source that travels through a set of rigid tandem and ovoid (T&O) applicators inserted into the anesthetized patient. The radially symmetric dose distribution emitted by conventional BT sources, however, results in the poor tumor coverage as shown in FIG. 40. The desired radiation dose to the tumor, shown as the red outline, is 100% of the prescribed radiation dose, which is clearly not being achieved in a large fraction of the tumor. Improved tumor coverage can be achieved with intensity modulated brachytherapy (IMBT), which uses shielding of the radiation source to achieve a better dose distribution. Improved tumor coverage obtained with IMBT can be expected to increase local tumor control probability in any applicable tumor, improving patient outcomes.

The feasibility of IMBT has been investigated and it has been demonstrated that IMBT could be delivered using radioisotopes and the Xoft (Sunnyvale, Calif.) Axxent electronic brachytherapy source, respectively, by collimating the source with high-density shields that create fan beams. The fan beam source is rotated inside the patient in a manner such that the amount of time the source spends irradiating a given direction is optimized to ensure better tumor coverage and better critical structure avoidance than conventional brachytherapy. Although both approaches support the potential benefits of IMBT, there are two major challenges associated with the rotating shield approach to IMBT delivery. First, rotating and verifying the location of a moving shield inside a curved applicator is non-trivial. Second, the delivery times associated with IMBT are increased relative to conventional BT. This is due to the loss of emitted radiation in the rotating shield, which must remove a large fraction, possibly around 90%, of the radiation in order to achieve an advantage over conventional BT. If the rotating fan beam accounts for only 10% of the radiation emitted by the BT source, with the rest lost in the shield, then delivering the same dose distribution as conventional BT will require at least ten times as long with rotating-shield IMBT. This is because the fan will have to be pointed in 10 directions and stay pointed in each direction for the same amount of time necessary to deliver an entire conventional BT plan, which loses 0% of the radiation due to shielding.

In another aspect, of the nearly 11,000 annual cases of newly-diagnosed cervical cancer in the U.S., about 45% (5,000) are of stage IB2 or higher. Cervical cancer of stage IB2 or higher has 5-year survival rates of up to about 70%, and 5-year survival and local control ranges from 0-20% and 18-48%, respectively, for stage IVA tumors. Such cancers typically are treated with a combination of chemotherapy, EBRT, and an intracavitary BT boost to the tumor. The advent of MRI-guided BT has revealed that the close proximity of the bladder, rectum, and sigmoid to the tumor restrict the radiation dose that can be delivered to the non-symmetric extensions of bulky (e.g., greater than about 40 cc) tumors with conventional BT, likely reducing the chances of local control. Tumor dose conformity for such bulky tumors can be significantly improved through the use of supplementary BT through interstitial needles, which is more invasive than intracavitary BT, may cause complications, and can add 35-70 minutes to the BT procedure. As increasing tumor dose using supplementary interstitial BT has improved cervical cancer outcomes relative to intracavitary BT alone, it can be expected that RSBT based on eBT could be a less-invasive alternative to intracavitary plus interstitial BT, while still improving patient outcomes relative to intracavitary BT alone.

Rotating shield brachytherapy (RSBT) is one particular implementation of IMBT that can enable enhanced tumor conformity of the BT dose distribution through use of a partially-shielded radiation source. RSBT was first described theoretically as a means of improving tumor conformity of brachytherapy dose distributions for single-catheter and multi-catheter treatments. In early studies, RSBT dose distributions were modeled from a partially-shielded radiation source with the dosimetric characteristics of ¹⁹²Ir, but shielded with an unknown material that provided a sufficient, hypothetically-low, transmission to enable RSBT to be beneficial. Although the ideal transmission for an RSBT shield is dependent on the clinical case and the emission angle, a shield transmission of 50% was shown to be unacceptable. Since the half-value layer of the gamma ray emissions from ¹⁹²Ir is about 2.5 mm, relatively few cancer sites are treatable with ¹⁹²Ir-based RSBT.

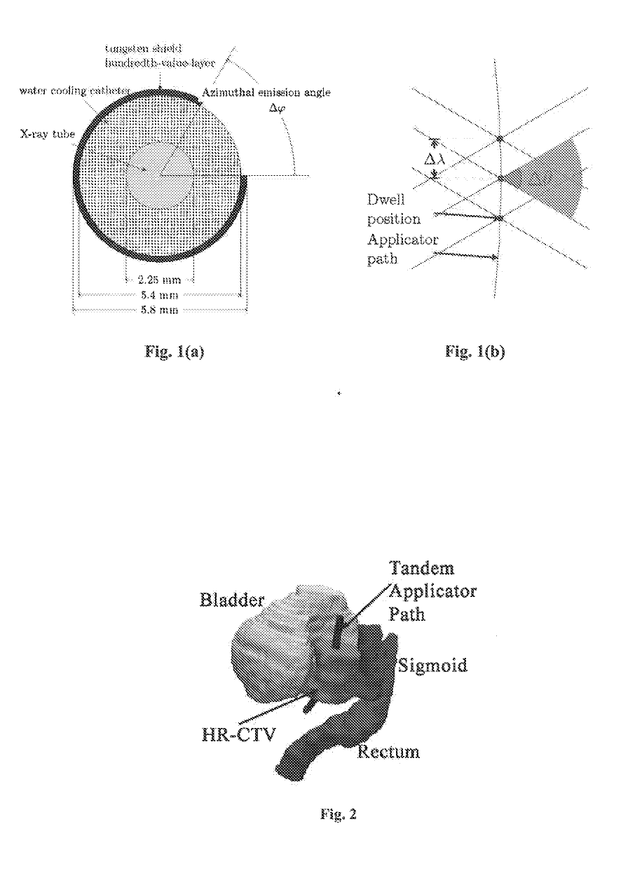

The advent of high-dose-rate electronic brachytherapy (eBT) sources such as the 40-50 kVp Xoft Axxent™ (Xoft Inc., Sunnyvale, Calif.) can enable RSBT in intracavitary applicators with diameters small enough to enable RSBT treatment of cervical cancer. The Xoft Axxent, shown in FIG. 1, is a 2.25 mm diameter x-ray tube, contained in a 5.4 mm diameter water cooling catheter, and emits x-rays with a hundredth-value-layer of 0.2 mm of tungsten. The Xoft Axxent, rotating shield, and applicator combination can provide an RSBT system with an overall diameter of less than 10 mm.

With RSBT, a shield partially-occludes the radiation source and rotates about the source in an optimized fashion, directing less radiation dose toward sensitive tissues than tumor tissues. However, for a given radiation source, single catheter RSBT treatment planning and delivery can be more costly and time consuming than conventional single-catheter BT treatment planning and delivery for multiple reasons. Since each source, e.g., a Xoft Axxent™ eBT source, has a finite lifetime, efficient usage of each source can be an important factor to ensure the treatment modality is cost-effective. Second, the treatment planning process for RSBT can be more time-consuming than that for conventional BT. Without wishing to be bound by theory and/or simulation, the number of optimization variables for RSBT is greater than that of conventional BT by a factor of K, where K is the number of allowed irradiation directions per dwell position. For example, it has been reported that multi-directional breast BT treatment planning and delivery times can take 120 minutes and 37 minutes, respectively, and conventional BT treatment planning and delivery times can both take only 5 minutes. Since patients tend to be under general or spinal anesthesia during BT treatment planning and delivery, prolonging any part of the treatment process is expensive and inefficient. Also, since BT radiation sources have a finite lifetime, efficient usage of each source is desirable to decrease the cost of BT therapy.

For patients treated with RSBT, it can be expected that treatment providers will have access to multiple shields with a range of emission angles. Without wishing to be bound by theory and/or simulation, the optimal emission angle for single-catheter, single-shield RSBT will be tumor-dependent, which can be illustrated clearly when a target with an ellipsoidal cross section and a catheter that passing through the center of mass of the target cross section is considered. For a target having an ellipsoidal cross section with a width of three times the height, an emission angle smaller than 180° will be desirable in order to treat the lateral tumor extensions without overdosing the normal tissue anterior and posterior to the tumor. For increasingly cylindrical targets, as the width and height of the tumor approach each other, larger emission angles become increasingly attractive, and the treatment times will decrease accordingly. For the limiting case of a target with a cylindrical cross section, the ideal source is an unshielded one, and the conventional BT case is desirable.

The choice of shield emission angle can be an important component in single-catheter, single-shield, RSBT planning. For difficult cases, determining the ideal shield angle for a given case by exhaustive treatment planning can be challenging due to the high computational cost, as the treatment planning time would scale with the number of available shields.

One or more embodiments of the disclosure can rectify such deficiencies by enabling a rapid RSBT emission angle selection method that can further enable the clinician to intuitively select an optimized balance between RSBT treatment time and dose distribution quality for a given clinical case. In one or more aspects, the shield angle selection method for several shield angles can require only half a minute of computational time beyond the time to generate a full RSBT treatment plan for a single shield angle. Other aspects, methods disclosed herein enable treatment providers to select the proper shield, balancing the delivery time and dose quality for each individual case, in a reasonable time with an REAS-generated Pareto plot as shown in FIG. 2. Each point on the Pareto plot represents the highest D₉₀ that can be achieved with the delivery time specified by its x-coordinate or the least delivery time required for achieving the D₉₀ specified by its y-coordinate. The emission angles for the delivery plans can also be indicated. Emission angles are invariant on the same curve segment, as plans located on the same curve segment are essentially the same plans with different scaling factors. In certain cases, the delivery time can be controlled below a certain time budget while minimizing the quality loss. In other cases, the dose quality (e.g., HR-CTV D₉₀) can be controlled above a certain goal while minimizing the delivery time. In one aspect, REAS can be of commercial value because it is a feasible method that can provide improvement over conventional S-RSBT treatment planning and delivery and can result in improved patient care. In another embodiment, the methods of the disclosure can enable shield selection methods requiring as little as about half of a minute of computational time beyond the time used to generate a full RSBT treatment plan for a single shield angle. Examples of cancers that can be treated more effectively with use of REAS in S-RSBT treatment comprise vaginal, cervical, endometrial, breast, lung, liver/bile duct, skin and/or prostate tumors.

One or more of the principles can be utilized in various therapeutic radiation treatments. In one aspect, an exemplary application of REAS is in the field of radiation oncology. More specifically, yet not exclusively, REAS can be utilized in conjunction with S-RSBT for the treatment of tumors that are not radially symmetric about a certain axis. In one example, REAS, in conjunction with S-RSBT, can overcome one or more limiting factors of treating cervical cancer tumors, which rarely are radially symmetric. In one embodiment, an electronic brachytherapy source, such as the Xoft Axxent™ can be inserted through a water cooling catheter and placed adjacent to or inside a target tissue using some applicator. Example applicators can include interstitial needles and intracavitary applicators. In S-RSBT as described herein, BT sources are not limited to electronic brachytherapy sources.

In one aspect, radiation source model and dose calculation can be accomplished by the following method. An RSBT beamlet, b_{i,j,k}(Δφ,Δθ), can be defined as the dose rate at the point \vec{r}ᵢ due to a shielded radiation source at dwell position \vec{s}ⱼ (i=0, . . . , J−1). As shown in FIG. 1(b), the shield has an azimuthal emission angle of Δφ and a zenith emission angle of Δθ. The irradiation direction of the beamlet is defined by φₖ, which is the lower of the two azimuthal angles defining the aperture: φₖ=(k mod K)δφ(k=0, . . . , K−1), where δφ=360°/K is the azimuthal step size between neighboring beamlets. The mod operation denotes modular arithmetic, enabling beamlet referencing with arbitrary integer k-values such that φ_{k+K+1}=φₖ₊₁. The upper azimuthal edge of beamlet k is located at angle φₖ+Δφ. The total dose delivered to point i from a shielded source with azimuthal and zenith emission angles of Δφ and Δθ, respectively, can be calculated, in one aspect, as a time-weighted sum of the appropriate beamlets over all dwell positions and emission angles:

$${d}_{i} ( Δ ϕ , Δ θ ) = {\sum }_{j = 0}^{J - 1} {\sum }_{k = 0}^{K - 1} {\dot{D}}_{i , j , k} ( Δ ϕ , Δ θ ) {t}_{j , k} , \qquad Eq . ( 1 )$$

where t_{j,k} is the dwell time, which is always greater than or equal to zero, for which the source is pointed in direction φₖ while it is located at dwell position j. The source step length along the source trajectory, Δλ, was set to 3 mm. As with φₖ, \dot{D}_{i,j,k}(Δφ,Δθ) and t_{i,k} are periodic functions of the index k with a period of K.

For exemplary and illustrative purposes only, RSBT source was assumed to be a 50 kVp Xoft Axxent™ with a 0.5 mm tungsten shield providing less than 0.01%, or effectively zero, transmission. RSBT beamlets were obtained by multiplying unshielded 3-D dose rate distributions obtained using a TG-43 dose calculation model by a binary function that was zero at all points blocked by the shield and unity at all other points. Thus, the point source approximation was used and the effects of shield emission angle size on the x-ray scatter component of the Xoft Axxent™ dose distribution were neglected. These approximations for this example are suitable since the emission angle selection method can be applied regardless of the accuracy of the beamlet calculation technique. The exact result of the method can have a slight, although currently unknown, dependence on the beamlet calculation technique. Also, for illustrative purposes, the shield emission angle selection problem is limited to azimuthal angles, and the zenith angle is held constant throughout the current work at Δθ=120°. Practically, the source emission direction would be controlled by rotating the shield about the source.

For exemplary and illustrative purposes only, two cervical cancer cases can be considered, and exemplary anatomy is shown in FIG. 2. The target region for each case is defined as the high-risk clinical target volume (HR-CTV), which was delineated by a radiation oncologist using the GEC-ESTRO recommendations and larger than 40 cm³ for both cases considered. The organs at risk (OARs) were the rectum, sigmoid colon, and bladder. Prior external beam radiotherapy (EBRT) doses of 45 Gy in 25 fractions of 1.8 Gy were delivered to the HR-CTV and OARs for both patients, which was accounted for in the BT treatment planning. The BT delivery was assumed to take place over 5 treatment fractions. Doses to the HR-CTV and OARs were expressed as equivalent doses in 2 Gy fractions (EQD2) using α/β values of 10 Gy and 3 Gy, respectively.

The RSBT and conventional (unshielded) BT treatment goal was to escalate tumor dose without exceeding the OAR tolerances. Specifically, the minimum dose to the hottest 90% (D₉₀) of the HR-CTV was maximized under the constraint that the minimum doses to the hottest 2 cm³ (D_{2cc}) of the rectum, sigmoid colon, and bladder could not exceed the tolerance doses of 75, 75, and 90 Gy₃, respectively. The Δφ-dependent treatment plan quality metrics were D₉₀ for the HR-CTV and the total delivery time.

In one aspect, the implementation of REAS can comprise the steps of generating beamlets by combining baseline beamlets, selecting a set of anchor plans, and generating a treatment plan.

In another aspect, generating beamlets can be accomplished by the following methods. The baseline beamlets can be defined as the beamlets generated using the baseline azimuthal angle, δφ. The baseline beamlets at a given dwell position j can be assumed to be non-overlapping, thus the shadows cast by the shields of neighboring beamlets (k and k+1 for a given dwell position j) do not overlap. An integer number, W (W>1), of neighboring baseline beamlets can be combined by superposition to produce a beamlet with a larger emission angle, Δφ_{W}=Wδφ, in one aspect, as follows:

$${\dot{D}}_{i , j , k} ( Δ {ϕ}_{W} , Δ θ ) = {\sum }_{p = 0}^{W - 1} {\dot{D}}_{i , j , k + p} ( δ ϕ , Δ θ ) , \qquad Eq . ( 2 )$$

generating a set of “W-beamlets.” Equation Eq. (2) is exact for the case of zero shield transmission, which is a safe assumption for the example of a Xoft Axxent™ shielded with 0.5 mm of tungsten.

In an example in which the W neighboring baseline beamlets, with indices from k to k+W−1, all share delivery times of t_{j,k}=τ, it follows from Equation Eq. (2) that the W neighboring beamlets can be replaced with a single beamlet with an emission angle Δφ_{W} and a delivery time of t_{j,k}^{W}=τ, where the t-superscript indicates that the delivery time is associated with a beamlet with an emission angle of Δφ_{W}. Conversely, a beamlet with an emission angle of Δφ_{W} and a delivery time of τ can be replaced with the baseline beamlets with indices between k and k+W−1, which will have delivery times of t_{j,k}¹=τ. Thus an entire set of dwell times associated with beamlets of emission angle Δφ_{W} can be written as a set of baseline dwell times (W=1), in one aspect, as follows:

$${t}_{j , k}^{W - 1} = {\sum }_{{k}^{′} = 0}^{K - 1} {t}_{j , {k}^{′}}^{W} Π ( \frac{( k - {k}^{′} ) mod K}{W} ) . \qquad Eq . ( 3 )$$

where

$$( \frac{a}{W} )$$

is unity when 0≦a≦W−1 (a is an integer) and zero otherwise. The purpose of the Π-function is to spread the dwell times from the Δφ_{W} emission angle beamlets over multiple baseline beamlets. The modular arithmetic in its argument makes Π a periodic function of k′ with period W. Equation Eq. (3) can, in one aspect, be simplified by changing summation indices for k′ to p=k−k′ as follows:

$${t}_{j , k}^{W \to 1} = {\sum }_{p = 0}^{K - 1} {t}_{j , k - p}^{W} Π ( \frac{p mod K}{W} ) = {\sum }_{p = 0}^{W - 1} {t}_{j , k - p}^{W} . \qquad Eq . ( 4 )$$

Since the sum over k′ in Equation Eq. (3) is over one period of a periodic function of k′, the summation over p in the middle expression of Equation Eq. (4) can be done over the same range, even after changing variables.

In yet another aspect, a treatment plan can be generated from anchor plans using the following methods. A treatment plan generated using the W-beamlets (W>0) and an in-house dose-volume optimizer can be denoted as \hat{P}^{W}, which has dwell times of \hat{t}_{j,k}^{W} and a dose distribution \hat{d}ᵢ^{W}. The baseline equivalent plan of \hat{P}^{W} is denoted as \hat{P}^{W→1}, which has dwell times of \hat{t}_{j,k}^{W→1} for baseline beamlets and the same dose distribution \hat{d}ᵢ^{W}. As the dose-volume optimization is a non-convex optimization problem and no polynomial algorithm exists, simulated annealing technique, in one aspect, can be applied to solve the dose-volume optimization. In order to make the simulated annealing efficient, initial solutions can be generated with a surface optimizer which uses a gradient-based least squares method to optimize the dose homogeneity on the HR-CTV surface. The simulated annealing can require 10˜20 minutes to converge even with initial guesses from the surface optimizer. Therefore, it is not practical to generate plans with all possible W-values under the time requirement of clinical practices. In order to overcome this obstacle, RSBT plans can be used to limit the number of calls to the optimizer.

In one aspect, an anchor plan \hat{P}^{W} for a given patient is the treatment plan generated with W-beamlets, by finding \hat{t}_{j,k}^{W}, which is the optimal t_{j,k}^{W} for (j=0, . . . , J−1, k=0, . . . , K−1). The baseline equivalent plan \hat{P}^{W→1} can then be obtained directly from \hat{P}^{W} without modifying the delivered dose distribution, \hat{d}ᵢ^{W}. Then, an expedient treatment plan \tilde{P}^{W′}, which has dwell times \tilde{t}_{j,k}^{W′}, is rapidly generated from an anchor plan \hat{P}^{W}, in one aspect, by solving the following optimization problem:

$$min {\sum }_{j = 0}^{J - 1} {\sum }_{k = 0}^{K - 1} {( {\hat{t}}_{j , k}^{W \to 1} - {\tilde{t}}_{j , k}^{{W}^{′} \to 1} )}^{2} • \\ s . t . {\tilde{t}}_{j , k}^{{W}^{′} \to 1} = {\sum }_{p = 0}^{W - 1} {\tilde{t}}_{j , k - p}^{{W}^{′}} \\ {\sum }_{j = 0}^{J - 1} {\sum }_{k = 0}^{K - 1} {\tilde{t}}_{j , k}^{{W}^{′}} \leq {T}^{max } \qquad Eq . ( 5 )$$

However, due to the inevitable error between \hat{t}_{j,k}^{W→1} and \check{t}ᵢₖ^{W′→1} in most real-world cases, \tilde{P}^{W′} may not be able to reproduce the dose distribution of \hat{P}^{W} perfectly. The plan quality tends to degenerate as W′ increases. As a result, expedient plan \tilde{P}^{W′} can be regarded as an approximation of dose-volume optimized plan \hat{P}^{W}, however, the approximation quality will decrease as W′ increases.

With the solution to Equation Eq. (5), \tilde{t}ᵢₖ^{W′} is then escalated to maximize D₉₀ in the HR-CTV. Tₘₐₓ is a constraint on the total delivery time of \tilde{P}^{W′} which can be imposed to reduce treatment time at the expense of HR-CTV D₉₀. Obtaining \tilde{P}^{W′} by solving the sequencing problem in Equation Eq. (5) enables a much faster result than by obtaining the full optimization needed to obtain \hat{P}^{W′}, since the problem concerns times only, rather than doses.

In one aspect, in order to balance the time cost spent on exhaustive re-optimization and the plan quality, a small set of anchor plans \hat{P}^{a}, \hat{P}¹⁴ and \hat{P}²⁴ can be selected. The corresponding azimuthal emission angles are 90°, 180° and 240°. Since the emission angles selected are, evenly-spaced among all possible emission angles, they can be considered as a sampling of the full-set of simulated-annealing optimized plans. The optimal sequencing algorithm can then be applied for each anchor plan and a Pareto-front generated, showing the trade-off between D₉₀ and delivery times for all possible W's.

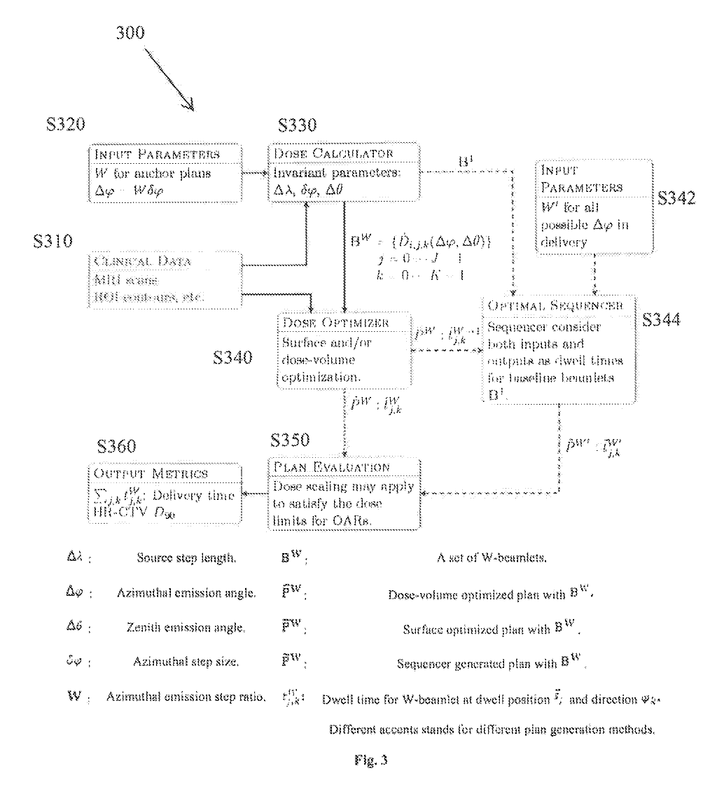

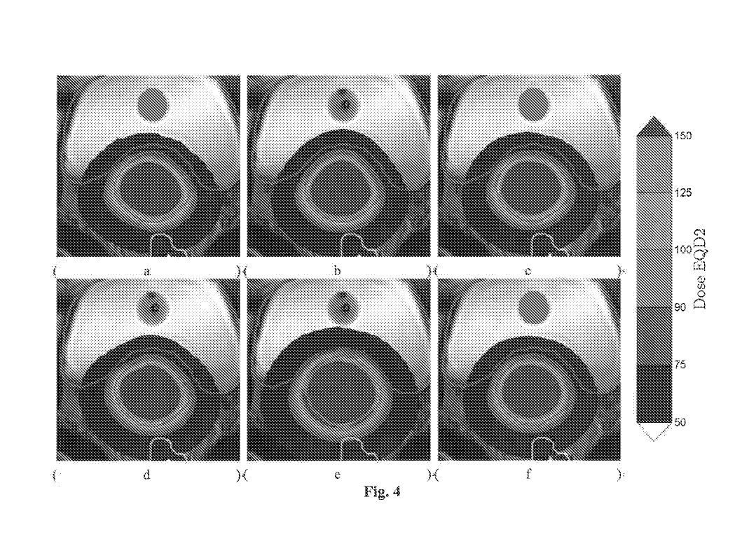

In view of the aspects described hereinbefore, an exemplary method that can be implemented in accordance with the disclosed subject matter can be better appreciated with reference to the flowchart in FIG. 3. For purposes of simplicity of explanation, the exemplary method disclosed herein is presented and described as a series of acts; however, it is to be understood and appreciated that the claimed subject matter is not limited by the order of acts, as some acts may occur in different orders and/or concurrently with other acts from that shown and described herein. For example, the various methods or processes of the subject disclosure can alternatively be represented as a series of interrelated states or events, such as in a state diagram. Moreover, when disparate functional elements implement disparate portions of the methods or processes in the subject disclosure, an interaction diagram or a call flow can represent such methods or processes. Furthermore, not all illustrated acts may be required to implement a method in accordance with the subject disclosure. Further yet, two or more of the disclosed methods or processes can be implemented in combination with each other, to accomplish one or more features or advantages herein described. It should be further appreciated that the exemplary methods disclosed throughout the subject specification can be stored on an article of manufacture, or computer-readable medium, to facilitate transporting and transferring such methods to computers for execution, and thus implementation, by a processor or for storage in a memory.

FIG. 3 is a flowchart of an exemplary method 300 for selecting an emission angle for use in S-RSBT in accordance with aspects of the subject disclosure. Also shown in FIG. 3 is an exhaustive replanning method (either simulated annealing or gradient-based) for comparison. A computer or computing device can implement (e.g., execute) exemplary method 300. In one aspect, a processor within or functionally coupled to the computer or computing device can be configured to execute computer-executable instructions and, in response to execution, the processor can carry out the various steps that comprise exemplary method 300. Similarly, yet not identically, the computer or computing device can execute the various methods, or portion(s) thereof, disclosed herein. The exemplary method 300 can comprise various steps. At step S310, receiving data indicative of a radiation treatment and topology of a region to be treated. At step S320, receiving input parameters for azimuthal emission angle and azimuthal emission step ratio for at least one and, preferably, three anchor plans. In an aspect, three anchor plans are needed to ensure that the generated plans are comparable to those that would be generated with less time-efficient planning methods. At step S330, calculating a dose. At step S340, optimizing the calculated dose using at least one of surface optimization and dose-volume optimization. At step S342, enumerating all possible input parameters. In an aspect, such a step can be done enumerating all possible positive integers that are less than or equal to 360°/δφ (the parameters supplied describe the azimuthal emission angles). At step S344, approximating the dose distribution of the anchor plan using beamlets from shields with azimuthal emission angle W′Δφ. At step S350, evaluating a treatment plan which may include dose scaling to satisfy dose limits for any organs at risk. At step S360, generating output metrics which may include generating a Pareto plot.

Steps S342 and S344 can be related to an optimal sequencing algorithm for REAS, which intends to reproduce the dose distribution of the anchor plan with beamlets that have larger azimuthal emission angles thus reduce the delivery time. The possible azimuthal emission angles were enumerated by step S342 to determine the best angle choice.

For further comparison, examples of three different planning methods are applied on two clinical cases for comparison. The two clinical cases are denoted Patient 1 and Patient 2, respectively. In FIG. 3, the exhaustive replanning method using a dose-volume optimizer, and the corresponding plans are denoted as \hat{P}^{W}; the exhaustive replanning using a surface optimizer, and the corresponding plans are denoted as P̅^{W}, and the exemplary method 300 for selecting an emission angle for use in S-RSBT in accordance with aspects of the subject disclosure as \tilde{P}^{W}.

For the example of treatment plan generation by the exhaustive replanning method using a dose-volume optimizer, each additional optimization takes about 10 minutes. In one example where the computational budget is limited to about 10 minutes to avoid too much time cost, a planning procedure with 32 plans for W=1˜32, takes about 7 hours to complete.

For the example of treatment plan generation by the exhaustive replanning method using a surface optimizer, the replanning procedure can take about 10 minutes to finish and the entire planning procedure can be completed in about 20 minutes.

For the example of treatment plan optimization via the exemplary method 300 for selecting an emission angle for use in S-RSBT in accordance with aspects of the subject disclosure, treatment plan generation requires about half of a minute beyond the generation of the anchor plans. Generating three anchor plans, in one aspect, takes about 40 minutes. In another aspect, the anchor plans can be generated in parallel and the entire planning procedure can be finished in about 20 minutes.

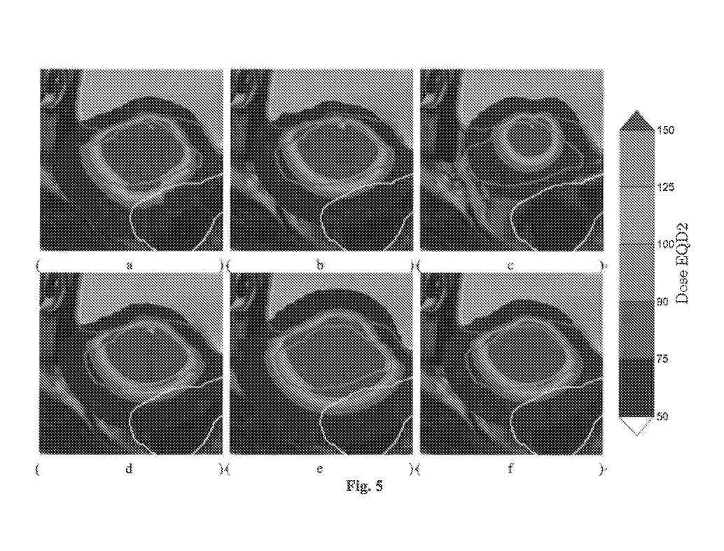

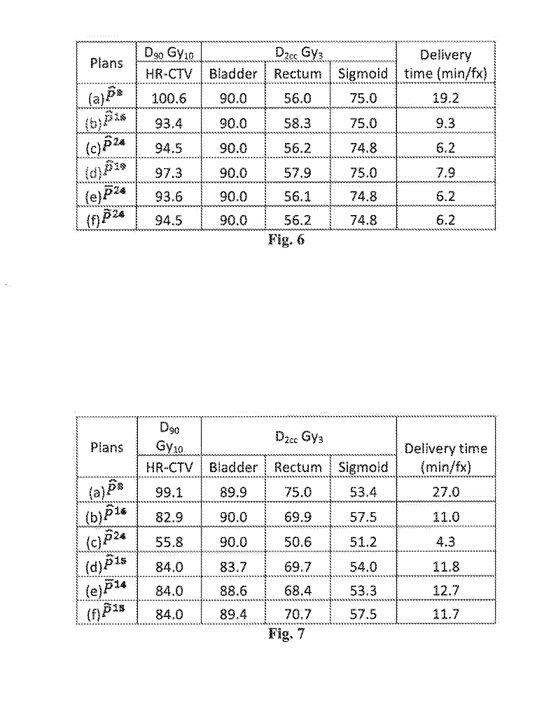

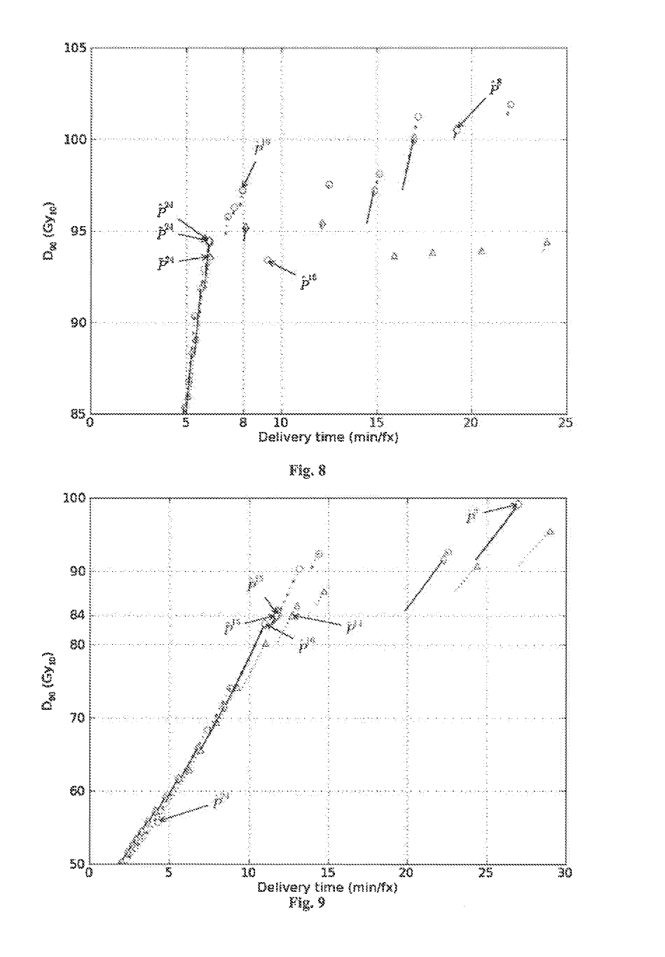

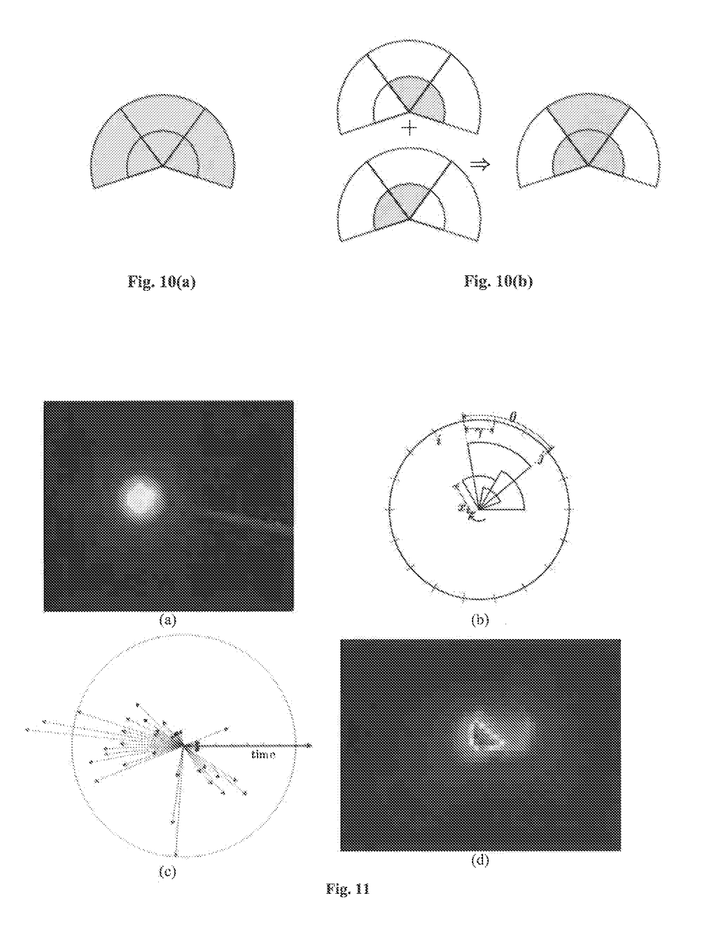

Visual comparisons of the dose distributions between the different plans described above are shown in FIG. 4 and FIG. 5. The corresponding quantitative comparisons are shown in FIG. 6 and FIG. 7 and the corresponding Pareto plots are shown in FIG. 8 and FIG. 9. For Patient 1, besides the anchor plans, the three additional plans (d)-(f) were selected as the optimal treatment plans to achieve minimal delivery time at the D₉₀ level 84 Gy₁₀. For Patient 2, the three additional plans (d)-(f) were selected as the optimal treatment plans to achieve the highest D₉₀ at the goal delivery time of 8 min/fx.

In certain embodiments, rapid emission angle selection can be achieved by combining dose-volume optimization and the sequencing algorithm, with setting either a goal for D₉₀ or a budget for the delivery time on the final Pareto plots. In one aspect, by selecting 3 anchor plans, sequenced plans can result in better approximations for dose-volume optimized plans compared with surface optimized plans, as shown in FIG. 8 and FIG. 9. In another aspect, the computational cost for sequencing algorithms can be negligible compared to the computational costs for dose calculations and optimizations. In another aspect, selection of the azimuthal emission angle Δφ can be case-dependent. In yet another aspect, smaller azimuthal emission angles do not necessarily result in a better dose distribution due to use of a fixed emission angle. If the larger emission angle is not a multiple of the smaller one, we cannot always expect getting a better dose distribution by using the smaller one. As illustrated by FIG. 10, suppose the ideal dose distribution is shown in (a), and it can be perfectly reproduce by set Δφ=3δφ. However, with a smaller emission angle Δφ=2δφ, it is impossible to perfectly reproduce the dose distribution, as shown in Fig. (b).

In yet another aspect, an Xoft Axxent electronic brachytherapy source shielded to less than about 1% transmission using less than about 0.2 mm of gold, tungsten, lead, or some other high-Z material is used. The shield design could, for example, be one of those shown in U.S. Pat. No. 7,686,755. The shield rotation can be accomplished by rotating the entire source wire inside the applicator, or rotating the shield about the source-containing catheter. In the present aspect, as illustrated in FIGS. 11(a-d), the source is partially shielded and the transmission rate at the shield is negligible or can be controlled at a low level. The dose distribution of a single beamlet through the shield is fan-like on a 2D slice with a certain thickness, as shown in FIG. 11(a). The color shows the difference of the dose contribution to different positions. The tumor surface can be equally divided into several divisions by angle in a polar system centering at the tandem point, with each division prescribed a desired dose, a maximum dose and a minimum dose it should receive. The shield can be rotated such that the fan-window opened in the shield can be selectively directed to any division or divisions. Typically, the fan window has an angle which can cover multiple adjacent divisions. Thus, each beamlet (corresponding to each direction the fan-window points to), will have a certain contribution to the surrounding tissues. FIGS. 11(b-c) illustrate an optimal dwelling time sequence for the fan-window to stop for the present aspect. As shown in FIG. 11(c), the case illustrated has 18 divisions, and the fan-window can cover 3 divisions at a time. The difference between the fans demonstrates the dwell-time difference for the fan-window when pointing to different divisions. The resulting dose distribution from FIG. 11(c) is illustrated in FIG. 11(d).

In an aspect, for the optimization objective, any of the following three options can be pursued: (i) minimizing the treatment time with all divisions receiving doses within the interval between the corresponding maximum and minimum (also referred to as the “MINTIME” problem); (ii) minimizing the total sum of errors within a given delivery time in addition to all constraints mentioned in (i) (also referred to as the “MINERR” problem); and (iii) minimizing the delivery time with the total sum of errors bounded in addition to all constraints mentioned in (i) (also called the “BALANCE” problem). It is a further aspect of this example to incorporate the combination of using an unshielded source together with the sequencing of the fan-window, which is equivalent with controlling the transmission rate through the shield, to gain further reduction of the delivery time.

A further feature of this example is the dual transformation. Although general integer programming problems are NP hard, which means that the global optimum cannot be guaranteed to be found in a short time, here the problems have a nice circular I's property in the constraint matrix which enables them to be converted to network problems and then solved in polynomial time. After the dual transformation, in one aspect, the MINTIME problem can be solved by using a parametric shortest path algorithm reported by Dorit Hochbaum, et al. in 2005. The MINERR and BALANCE problems can also be solved by applying the net surface detection technique by Wu, et al. in 2002.

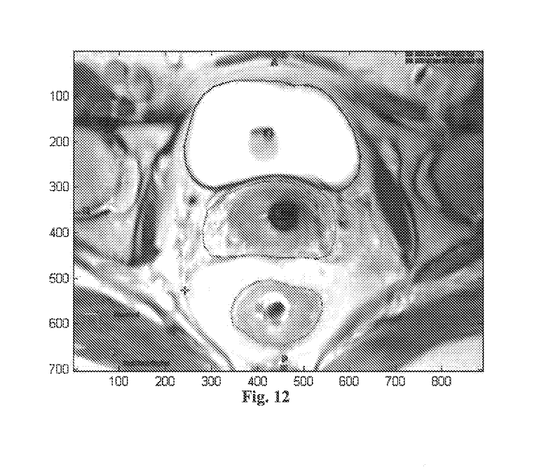

In one aspect, after the sequencing algorithm is completed, the outputs can be fed back to create new dose distributions and these new dose distributions are subjected to a dose rescale to ensure the tumor is not underdosed as long as the OARs are not overdosed according to GEC-ESTRO recommendations. The present method can be verified with a 2D slice from clinical cervical cancer case, as shown in FIG. 12. The tumor surface (contour) can be equally divided into 90 divisions by angle. The 3 contoured objects from top to bottom are bladder, tumor site and rectum. For the dose calculation, a single raytrace method that accounts for polyenergetic source model, attenuation in the patient, and inverse square law can be used. Scatter and anisotropy are not accounted for in this example.

The verification was performed by using a software implementation of the algorithms of this example with a computer workstation, and every single optimization procedure was performed within about 2 seconds. Testing of the software implementation of the algorithms can be under several different settings (varying fan angles, toggle between using combination with unshielded source or not) to see the impact of different parameters.

In order to simplify the verification process, all the doses were normalized such that the prescription dose is 100 units and, for simplicity in this 2-D example, it can also be assumed that each pixel in the image corresponds to 0.02cc volume. Under this assumption, we will get 32.8cc for the tumor, 59.98cc for the bladder and 16.7cc for the rectum. By following the GEC-ESTRO recommendations for DVH parameters, all the constraints for the dose rescaling procedure specifically for our test case are obtained:
1. At least 90% percent of the tumor should receive 100% prescription dose;
2. 2cc of the bladder tissue should receive no more than 86% prescription dose;
3. 2cc of the rectum tissue should receive no more than 58% prescription dose.

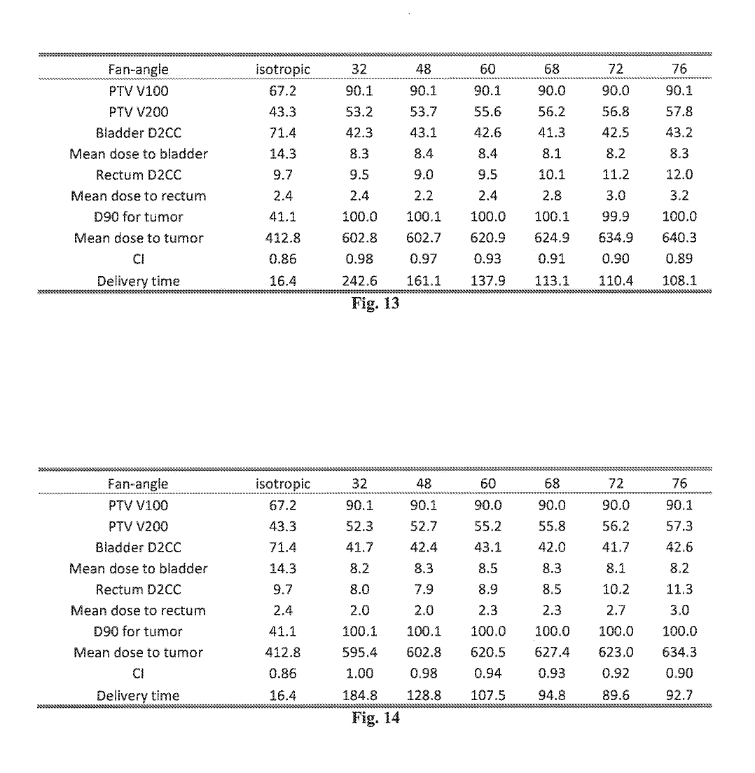

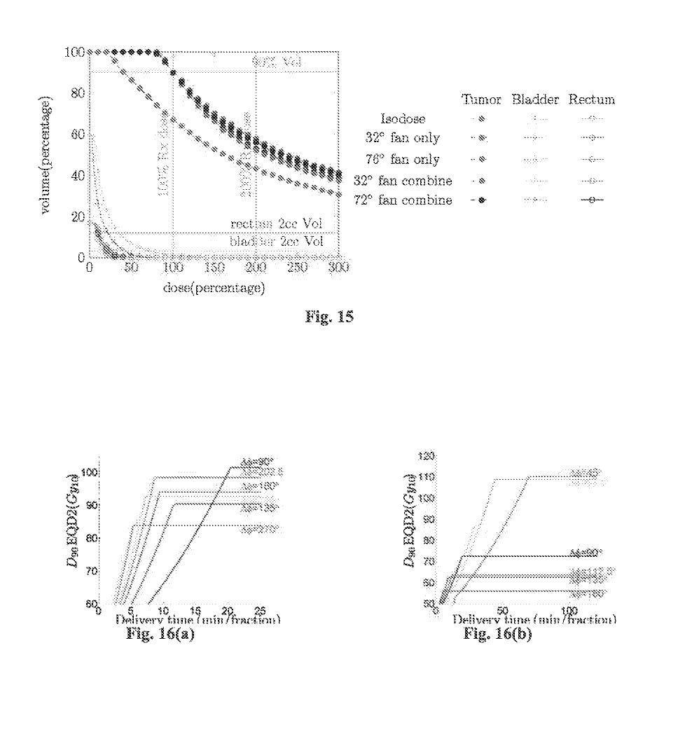

As presented in FIGS. 13-14, the results indicate that IMBT can noticeably increase the dose distribution quality compared with conventional isotropic radiation source, as the conventional method will inevitably underdose the tumor within the safety constraints. However, with IMBT, the price paid for increasing the dose distribution quality is a longer delivery time. The best result in FIG. 13 is that the IMBT delivery uses about 5.6 times more than conventional brachytherapy. It is also shown that, as the fan angle increases, both the delivery time and the conformity indices decrease. However, the treatment time cannot be decreased by increasing the fan-angle as it would lead to problems of feasibility. In the case we show in FIG. 15, a solution with all constraints satisfied cannot be determined when the fan-angle is 80° or more. This can be because the problem will degenerate to the conventional isotropic problem when the fan-angle increased to its maximum 360°. As shown in FIG. 15, the delivery time can be further reduced while suffering little from the dose distribution with the combination of using a unshielded source. Note that the maximum allowed percentage of dose from unshielded source while keeping the problem feasible is used here. Here, 4.46 times more delivery time compared to conventional isotropic source is achieved.

In yet another example, an efficient inverse planning system is need for making RSBT practical given the time constraints imposed by the anesthetized patient. Existing dose optimization methods can take a long time to reach a desired solution (e.g., simulated annealing), or can compromise the quality of the plan (e.g., using surface-based optimization instead of dose-volume based optimization). In this example, in order to optimize the balance between the dose quality and the delivery time, a rapid method for the dose quality for each possible delivery configuration is provided. However, the delivery configuration (i.e. azimuthal emission angle δφ in this study) can vary significantly between cases, and dose-volume optimization with simulated annealing can take about 20 minutes for each delivery configuration. Therefore, it is unlikely to make repeated dose-volume optimization for each delivery configuration in clinical practices. In one aspect, this problem is addressed by decoupling the dose optimization and the plan delivery. For each of the two exemplary clinical cases used in this study, an anchor plan which maximizes the minimum dose received by the hottest 90% of the tumor (D₉₀) to the tumor but with infeasible delivery time can be generated. This anchor plan can utilize micro-azimuthal-angle δφ as the azimuthal emission angle, and the dose-volume optimization can be accomplished with simulated annealing. Solutions generated from surface-based optimization can be used as initial solutions to speed up the optimization procedure. The whole optimization procedure can take about 20 minutes to finish. The RSBT emission angle selector can determine the optimal emission angle for a given clinical case by combining neighboring beamlets from the anchor plan to form the beamlets corresponding to larger emission angles Δφ=Wδφ(W>1). The delivery times for each beam direction for the larger emission angles can be determined by efficiently solving a globally-optimal quadratic programming problem that closely reproduces the angular distribution of beam intensities from the anchor plan. The dosimetric quality assessment for each emission angle Δφ can take less than about one second.

In an aspect, a Pareto plot of the dosimetric plan quality metric, such as D₉₀ versus the delivery time, is generated for the treatment provider. Examples of such an aspect are shown in FIGS. 16(a-b). For each patient, points on a Pareto front (black curve) are generated for each available azimuthal emission angle. A subset of the emission angles is shown for the sake of clarity. Based on these Pareto plots, an appropriate azimuthal emission angle for patient 1 is Δφ=202.5° and it is superior to the choice of 180° and 135°. For patient 2, Δφ=67.5° would be a good choice for balancing the dose quality and delivery time in this example. It follows that, the treatment provider can determine the most appropriate emission angle for a given clinical case by considering the tradeoff between the dose quality and the delivery time. In this example, two cervical cancer cases were considered to test the emission angle selection algorithm and associated methods disclosed herein. The RSBT system can be a Xoft Axxent™ electronic brachytherapy source with a 0.2 mm tungsten shield. The goal of each treatment plan was to maximize tumor D₉₀ while respective the GEC-ESTRO recommended constraints on the D_{2cc} values to the bladder, rectum, and sigmoid colon of 90, 75, and 75 Gy₃, respectively, which includes the dose from external beam radiotherapy (EBRT). For the purpose of comparison, the quantitative dosimetric plan qualities and delivery times for several different delivery configurations were calculated, including:

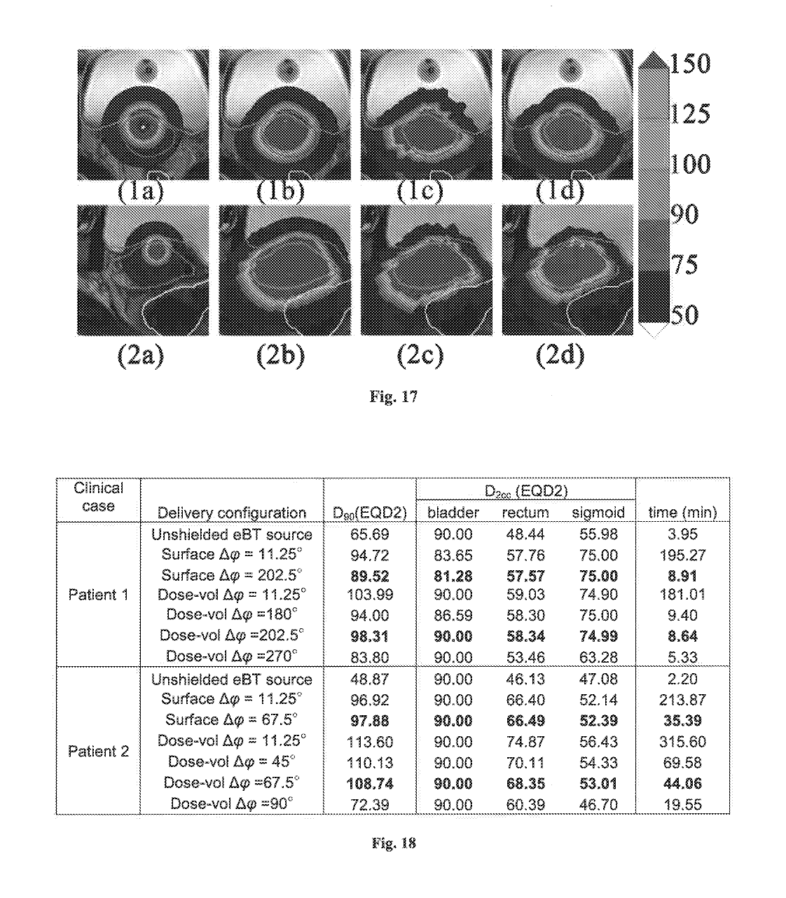

FIG. 17 illustrates exemplary dose distributions for selective delivery configurations for two exemplary clinical cases. FIG. 17(1a) and FIG. 17(2a) illustrate the delivery of doses with an unshielded eBT source, optimized with a surface-based optimizer for the two patients. A delivery using RSBT with all possible azimuthal emission angles, optimized with surface-based optimizer are shown for the two patients in FIG. 17(1b) and FIG. 17(2b). Patient 1 had the delivery applied at azimuthal emission angle Δφ=202.5° and Patient 2 at Δφ=67.5°.

FIG. 17(1c) and FIG. 17(2c) illustrate a delivery using RSBT with anchor plans optimized by both surface-based and dose-volume based optimizer, where azimuthal delivery angle Δφ equals the micro-azimuthal-angle δφ=11.25° in this study. FIG. 17(1d) and FIG. 17(2d) illustrate the delivery using RSBT with all possible azimuthal emission angles, using RSBT emission angle selector based on the dose-volume optimized anchor plan. (Patient 1 with Δφ=202.50) and FIG. 17(2d) (Patient 2 with Δφ=67.5°).

Appendix C illustrates yet another example of optimal emission angle selection in rotating shield BT illustrating another example of decoupling the sequencing procedure from dose optimization as described in the present disclosure.

Appendix D illustrates yet another example of a combinatorial optimization method for sequencing the rotating shields in IMBT described in the present disclosure.

Appendix E illustrates yet another example of a combinatorial optimization method for sequencing the rotating shields in IMBT described in the present disclosure.

In other embodiments of the disclosure, a method for facilitating the procedure of determining the delivery in dynamic rotating-shield brachytherapy (D-RSBT), called optimal sequencing for D-RSBT (OSD) is provided. D-RSBT, as an intensity modulation technique, can enable the delivery of deliberately non-symmetric, tumor-conformal, dose distributions that would be impossible to deliver with conventional radiation sources by using a source encapsulated by two partial shields. Using a small emission angle (referred as azimuthal step angle δφ), the dose optimizers can achieve high tumor dose conformity. However, the time necessary to treat a tumor with RSBT is inversely proportional to the shield emission angle, thus small emission angle shields increase delivery times. By decoupling the delivery step from the optimization step, OSD can enable treatment providers to make a quick decision on the trade-off between the dose quality and the delivery time based on the time budget for treatment and designated goal of plan quality. OSD is a method that can enable a reduction in the treatment time necessary to deliver an RSBT treatment.

Further, S-RSBT performs well in clinical cases where the targets are regularly shaped, however, when the shapes of targets become increasingly irregular, the REAS techniques applied to S-RSBT as described above may become less useful. Without wishing to be bound by theory and/or simulation, approximating a wavy curve is generally harder than approximating a smoother curve using the same window with fixed size. In one aspect, by using a coupled partial-shield, the D-RSBT applicator can enable adjustment of the emission angle even during the delivery, as shown in FIG. 20. Therefore, in those cases, D-RSBT can enable better delivery plans than S-RSBT as it can have a much wider selection of beams than S-RSBT.

Unlike S-RSBT, the delivery time is the primary factor influencing the dose quality in D-RSBT. As D-RSBT allows use of any azimuthal emission angle less than 180°, the OSD algorithm can enable improved reproduction of the dose distribution of anchor plans while reducing the delivery times to produce the dose distribution given an adequate delivery time budget. Even when presented with a suboptimal delivery time budget, the plan generated by OSD algorithm can be of comparable or improved quality compared to one generated using REAS as the set of beams used in S-RSBT (with azimuthal emission angle less than 180°) are a subset of the beams used in D-RSBT. Thus, in one aspect of the present disclosure, D-RSBT using OSD algorithm can enable the most accurate approximation of the anchor plans under any given delivery time budget.

One or more embodiments of the present disclosure provide for a method for optimal sequencing in D-RSBT that can enable a treatment provider to make a rapid and appropriate shield selection while (i) controlling the delivery time below a certain time budget to minimize loss of dose quality; or (ii) controlling the dose quality (e.g. HR-CTV D₉₀) above a certain goal while minimizing the delivery time. In another aspect, a treatment provider can balance the delivery time and the dose quality for each individual clinical case. Other aspects of the present methods enable treatment providers to select the proper shield, balancing the dose quality and the delivery time for each individual clinical case, in a reasonable time with an OAS-generated Pareto plot, an example of which is shown in FIG. 21. Each point on the Pareto plot represents the highest D₉₀ that can be achieved with the delivery time specified by its x-coordinate or the least delivery time required for achieving the D₉₀ specified by its y-coordinate.

In one aspect, radiation source model and dose calculation can be accomplished by the following method. An RSBT beamlet, D_{i,j,k}(Δφ,Δθ), can be defined as the dose rate at the point \vec{r}ᵢ due to a shielded radiation source at dwell position \vec{s}ᵢ (j=0, . . . , J−1). As shown in FIG. 20, the shield has an azimuthal emission angle of Δφ and a zenith emission angle of Δθ. The irradiation direction of the beamlet is defined by φₖ, which is the lower of the two azimuthal angles defining the aperture: φₖ=(k mod K)δφ(k=0, . . . , K−1), where δφ=360° and K is the azimuthal step size between neighboring beamlets. The mod operation denotes modular arithmetic, enabling beamlet referencing with arbitrary integer k-values such that φ_{k+K+1}=φₖ₊₁. The upper azimuthal edge of beamlet k is located at angle φₖ+Δφ. The total dose delivered to point i from a shielded source with azimuthal and zenith emission angles of Δφ and Δθ, respectively, can be calculated, in one aspect, as a time-weighted sum of the appropriate beamlets over all dwell positions and emission angles:

$${d}_{i} ( Δϕ , Δθ ) = {\sum }_{j = 0}^{J - 1} {\sum }_{k = 0}^{K - 1} {\dot{D}}_{i , j , k} ( Δϕ , Δθ ) {t}_{j , k} . \qquad Eq . ( 6 )$$

where t_{j,k} is the dwell time, which is always greater than or equal to zero, for which the source pointed in direction φₖ while it is located at dwell position j. The source step length along the source trajectory, Δλ, was set to 3 mm. As with φₖ, \dot{D}_{i,j,k}(Δφ,Δθ) and t_{j,k} are periodic functions of the index k with a period of K.

In another aspect, generating beamlets can be accomplished by the following methods. The baseline beamlets can be defined as the beamlets generated using the baseline azimuthal angle, δφ. The baseline beamlets at a given dwell position j can be assumed to be non-overlapping, thus the shadows cast by the shields of neighboring beamlets (k and k+1 for a given dwell position j) do not overlap. An integer number, W(W>1), of neighboring baseline beamlets can be combined by superposition to produce a beamlet with a larger emission angle, Δφ_{W}=Wδφ, in one aspect, as follows:

$${\dot{D}}_{i , j , k} ( {Δϕ}_{W} , Δθ ) = {\sum }_{p = 0}^{W - 1} {\dot{D}}_{i , j , k + p} ( δϕ , Δθ ) . \qquad Eq . ( 7 )$$

generating a set of “W-beamlets.” Equation (7) is exact for the case of zero shield transmission, which is a safe assumption for the example of a Xoft Axxent™ shielded with 0.5 mm of tungsten.

In an example in which the W neighboring baseline beamlets, with indices from k to k+W−1, all share delivery times of t_{j,k}=τ, it follows from Equation (7) that the W neighboring beamlets can be replaced with a single beamlet with an emission angle Δφ_{W} and a delivery time of t_{j,k}^{W}=τ, where the t-superscript indicates that the delivery time is associated with a beamlet with an emission angle of Δφ_{W}. Conversely, a beamlet with an emission angle of Δφ_{W} and a delivery time of T can be replaced with the baseline beamlets with indices between k and k+W−1, which will have delivery times of t_{j,k}¹=τ. Thus an entire set of dwell times associated with beamlets of emission angle Δφ_{W} can be written as a set of baseline dwell times (W=1), in one aspect, as follows:

$${t}_{j , k}^{W \to 1} = {\sum }_{{k}^{′} = 0}^{K - 1} {t}_{j , {k}^{′}}^{W} ( \frac{( k - {k}^{′} ) mod K}{W} ) , \qquad Eq . ( 8 )$$

where

$$( \frac{a}{W} )$$

is unity when 0≦a≦W−1 (a is an integer) and zero otherwise. The purpose of the Π-function is to spread the dwell times from the Δφ_{W} emission angle beamlets over multiple baseline beamlets. The modular arithmetic in its argument makes Π a periodic function of k′ with period W. Equation (8) can, in one aspect, be simplified by changing summation indices for k′ to p=k−k′ as follows:

$${t}_{j , k}^{W \to 1} = {\sum }_{p = 0}^{K - 1} {t}_{j , k - p}^{W} ( \frac{p mod K}{W} ) = {\sum }_{p = 0}^{W - 1} {t}_{j , k - p}^{W} . \qquad Eq . ( 9 )$$

Since the sum over k′ in Equation (8) is over one period of a periodic function of k′, the summation over p in the middle expression of Equation (9) can be done over the same range, even after changing variables.

In yet another aspect, a treatment plan can be generated from anchor plans using the following methods. A treatment plan generated using the W-beamlets (W>0) and in-house dose-volume optimizer can be denoted as \hat{P}¹ (also referred as the “baseline anchor plan”), which has dwell times of \hat{t}_{j,k}¹ and a dose distribution \hat{d}ᵢ¹. As the dose-volume optimization is a non-convex optimization problem and no polynomial algorithm exists, simulated annealing techniques, in one aspect, can be applied to solve the dose-volume optimization. In order to make simulated annealing efficient, initial solutions can be generated with a surface optimizer which uses a gradient-based least squares method to optimize the dose homogeneity on the HR-CTV surface. The simulated annealing can require about 10-20 minutes to converge, even with initial guesses from the surface optimizer.

In another aspect, baseline anchor plans can have a high dose quality due to their small emission angle; however, they also have impractical delivery times as the plans only utilize the W-beamlets.

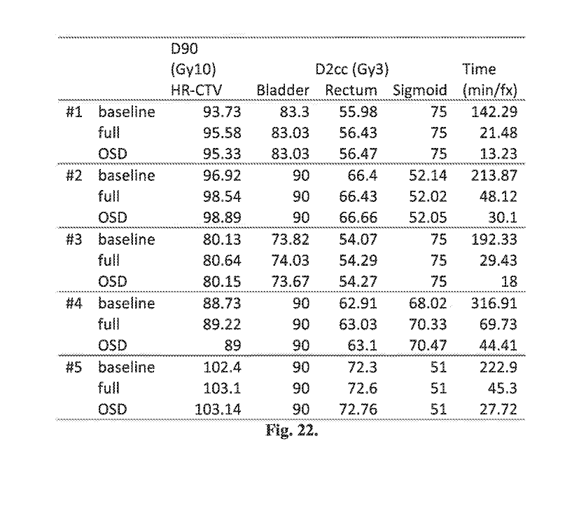

In other aspects, in order to utilize the full range of possible W-beamlets, there are two different options: First, delivery plans for D-RSBT can be generated directly by using the union of all W-beamlets that satisfies Wδφ≦180° in the dose optimizer. However, that this option can place high demand on available computational resources (e.g., memory and CPU time) and the simulated annealing optimizer can, in one aspect, be rendered unable to compute due memory overflow (e.g., on a 4 GB workstation); the computational time for the gradient method can increase by about 5 times; and, meanwhile, delivery time can be decreased. Second, delivery plans for D-RSBT can be generated using the baseline anchor plan and applying OSD which can access all possible W-beamlets to reproduce the dose distribution of the baseline anchor plan within a given delivery time. This option places less demand on available computational resources. In another aspect, as shown in FIG. 22, using the full range of W-beamlets in the optimizer can enable achievement of approximately the same quality as the baseline anchor plans with a shorter delivery time; and the OSD algorithm can enable further reductions of about 40% in the delivery time. Five exemplary clinical cases are used for this comparison. For each exemplary clinical case, a baseline anchor plan was first computed (using gradient-based optimizer as the simulated annealing optimizer cannot finish for the latter optimization with a full range of beamlets) and is referred to as “baseline” in the table. Next, another delivery plan with full range of beamlets was computed with the same gradient-based optimizer and is referred to as “full” in the table. Then, an OSD algorithm is applied to the plan generated from the full delivery and is referred to as “OSD” in the table.

The OSD algorithm, in one aspect, can be formulated through Equation (10).

$$min {\sum }_{j = 0}^{J - 1} {\sum }_{k = 0}^{K - 1} {( {\hat{t}}_{j , k}^{1} - {\tilde{t}}_{j , k}^{\to 1} )}^{2} \\ s . t . {\tilde{t}}_{j , k}^{\to 1} = {α}_{j , k} - {b}_{j , k} + {a}_{j , K - 1} , 0 \leq k < {W}_{max} \\ {\tilde{t}}_{j , k}^{\to 1} = {a}_{j , k} - {b}_{j , k} , {W}_{max} \leq k < K \\ {a}_{j , k} \leq {a}_{j , k + 1} , {b}_{j , k} \leq {b}_{j , k + 1} , {a}_{j , k} \leq {a}_{j , k + 1} , {a}_{j , 0} \geq 0 \\ {\sum }_{j = 0}^{J - 1} {a}_{j , k - 1} \leq {T}^{max} \qquad Eq . ( 10 )$$

In Equation (10), \tilde{t}_{j,k}^{→1}, stands for the dwell time of the baseline equivalent of the OSD generated plan, a_{j,k} and b_{j,k} are related to the time point when the tailing and leading field edges pass the direction kδφ at dwell position j. T^{MAX} stands for the delivery time budget and can be used to control the balance between dose quality and the delivery time.

With the solution to Equation (10), \tilde{t}_{j,k}^{→1} is then scaled to ensure that the constraints on the minimum doses to the hottest 2 cm³ (D_{2cc}) of OARs do not exceed the tolerance. Obtaining the OSD sequenced plan by solving the sequencing problem in Equation (10) can require less computational resources than all the dose optimization problems above, since the problem concerns times only, rather than doses.

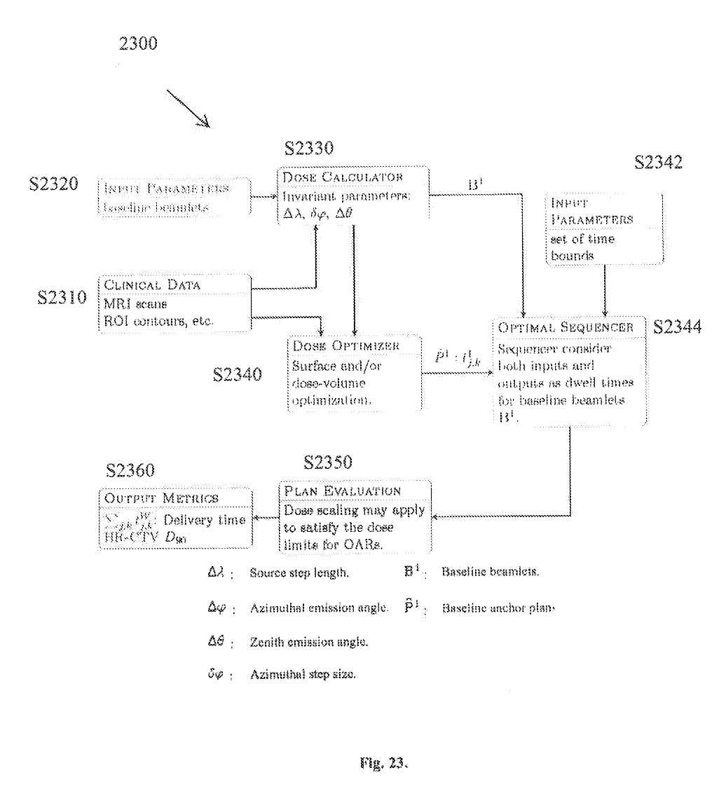

FIG. 23 is a flowchart of an exemplary method 2300 for using OSD in D-RSBT treatment planning. Also shown in FIG. 23 is an exhaustive replanning method (either simulated annealing or gradient-based) for comparison. A computer or computing device can implement (e.g., execute) exemplary method 2300. In one aspect, a processor within or functionally coupled to the computer or computing device can be configured to execute computer-executable instructions and, in response to execution, the processor can carry out the various steps that comprise exemplary method 2300. Similarly, yet not identically, the computer or computing device can execute the various methods, or portion(s) thereof, disclosed herein. The exemplary method 2300 can comprise various steps. At step S2310, receiving data indicative of a radiation treatment and topology of a region to be treated. At step S2320, receiving input parameters for azimuthal emission angle and azimuthal emission step ratio for at least one anchor plan. At step S2330, calculating a dose. At step S2340, optimizing the calculated dose using at least one of surface optimization and dose-volume optimization. At step S2342, enumerating all possible input parameters, which are a set of time bounds. In an aspect, such parameters are the delivery time budgets. At step S2344, approximating the dose distribution created from S2340, which is made subject to a delivery time constraint or budget from S2342. To find a balance between the dose quality and the delivery time, S2342 enumerates a set of different time budges, and the delivery efficiency curve is generated accordingly. At step S2350, evaluating a treatment plan which may include dose scaling to satisfy dose limits for any organs at risk. At step S2360, generating output metrics which may include generating a Pareto plot.

In one aspect, use of the OSD technique can result in significant reductions in delivery time relative to the baseline anchor plans without significantly impacting dose quality.

In another aspect, the time cost of OSD can be negligible (e.g., a few seconds). In another aspect, compared to S-RSBT, OSD generated plans can have improved quality for cases with irregular geometries.

In another example of the methods of this disclosure, beamlets can be generated using a dose calculator and the anchor plans can be computed using a dose-volume based optimizer (using simulated annealing). The optimizer can maximize the D₉₀ to HR-CTV while keep the D_{2cc} of the rectum, sigmoid colon, and bladder below 75, 75, and 90 Gy₃, respectively. A surface based optimizer (using gradient method) can be applied first to obtain an initial solution in order to speed up the subsequent dose-volume optimization. In this aspect, it can be shown that: (i) dose-volume optimization can increase the D₉₀ for about 10 Gy₁₀ compared to surface optimization with an extra computation time of about 10-20 min.; and (ii) without the initial solutions from the surface optimization, the dose-volume optimization can take over 5 hours to converge.

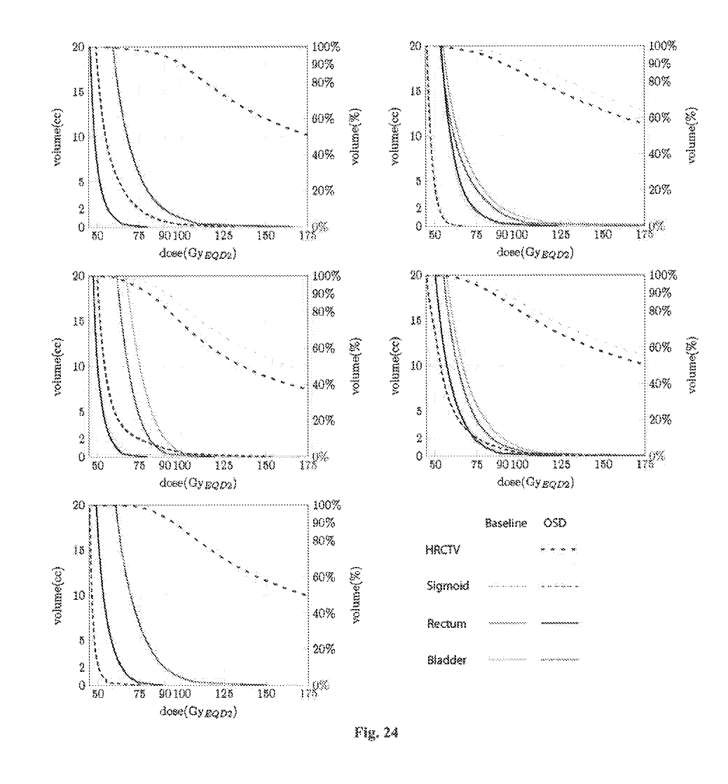

The OSD algorithm of the present disclosure can also be referred to as circular integral block decomposition (CIBD). Exemplary results of OSD are shown in FIG. 24 and FIG. 25.

In an aspect, a globally-optimal algorithm based on combinatorial optimization technique that balances the trade-off between treatment plan quality and delivery time is presented and can enable efficient D-RSBT delivery. In another embodiment, a CIBD problem can be configured to seek for an optimal set of circular blocks that stacks up to approximate a given reference integral function defined on a circular interval. This problem can be an effective model of the radiation dose delivery in D-RSBT. One challenge can lie in the circularity of the problem domain and the maximum length constraint of the circular blocks. In one aspect, an efficient polynomial time algorithm for solving the CIBD problem can be provided, enabling formulation of the CIBD problem as a convex cost integer dual network flow. In another aspect, implementation of the CIBD algorithm can run relatively fast and can produce promising D-RSBT treatment plans.

In one aspect, a CIBD problem can be provided and two integer parameters w>0 and H>0, and a non-negative integral function t that can be defined on a circular interval C=[0, n−1]. A circular window function ƒₖ(x), with

$${f}_{k} ( x ) = \{ \begin{matrix}{h}_{k} , & if x ∈ {ℐ}_{k} ⋐ C , \\ 0 , & otherwise ,\end{matrix}$$

where hₖ>0 is an integer constant and | [symbol] ₖ|≦w. can be provided. In one aspect, the CIBD problem can be utilized to find a set of circular window functions ƒₖ(x) that approximates the given function t by tiling them up and the total height of the window functions Σhₖ≦H.

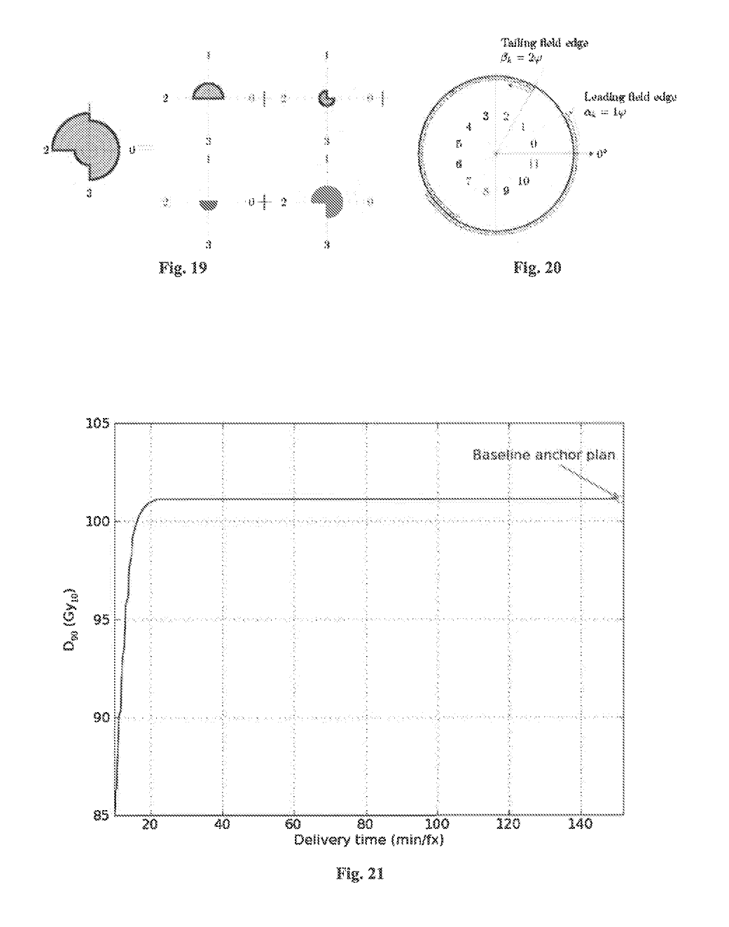

As shown by FIG. 19, a function t can be defined on a circular interval with n=4, namely xε{0, 1, 2, 3} (CCW) and t(x)={4, 5, 2, 4}. In one aspect, the function can then be perfectly decomposed (error-free) to a set of 4 circular window functions, denoted as B={<0, 2, 2>, <1, 0, 1>, <2, 0, 1>, <3, 2, 2>}, where each triplet <aₖ, bₖ, hₖ> represents a circular window function, with

$${f}_{k} ( x ) = \{ \begin{matrix}{h}_{k} , & if {a}_{k} < {b}_{k} , x ∈ {ℐ}_{k} = [ {a}_{k} , {b}_{k} - 1 ] \\ {h}_{k} , & if {a}_{k} \geq {b}_{k} , x ∈ {ℐ}_{k} = [ {a}_{k} , n - 1 ] ⋃ [ 0 , {b}_{k} - 1 ] \\ 0 , & otherwise\end{matrix} \qquad ( 1 )$$

Throughout this disclosure, circular window functions can also be referred to as a “block.” In one aspect, the maximal window size w among those blocks is 3 and the total height of all window functions is Σhₖ=6. In yet another aspect, function yield by tiling up all window functions in B can be defined as

$${ℱ}_{B} ( x ) = {\sum }_{{f}_{k} ∈ B , x ∈ {ℐ}_{k}} {h}_{k} \qquad ( 2 )$$

and ∀xε[0,n−1], [symbol] _{B}(x)=t(x).

In certain aspects, due to the constraint Σhₖ≦H, an exact decomposition of t may not be found. In one aspect, the CIBD problem can be defined as the following optimization problem:

$$min ɛ ( B ) = {\sum }_{x = 0}^{n - 1} {( {ℱ}_{B} ( x ) - t ( x ) )}^{2}$$

$$s . t . 1 \leq {b}_{k} - {a}_{k} \leq w or 1 \leq {b}_{k} + n - {a}_{k} \leq w , k ∈ [ 1 , \vert B \vert ]$$

$${\sum }_{k = 1}^{\vert B \vert} {h}_{k} \leq H$$

$${a}_{k} , {b}_{k} ∈ [ 0 , n - 1 ] , {h}_{k} > 0 , {a}_{k} , {b}_{k} , {h}_{k} ∈ ℤ , k ∈ [ 1 , \vert B \vert ]$$

Such CIBD problems can arise in the state-of-the-art Dynamic Rotating-Shield Brachytherapy (D-RSBT), which is another intensity-modulation technology for delivering radiation dose in brachytherapy.

In D-RSBT, the radiation source is partially-covered by a multi-layered radiation-attenuating shield, forming directed apertures called beamlets by rotating the field edges as illustrated in FIG. 20. The leading and tailing field edge can rotate independently, stopping at n discrete positions that can be distributed evenly along the circle. Each beamlet can be defined by the directions of the leading and tailing field edges with their rotation angles relative to some reference angle 0°.

In one aspect, for any known set of beamlets, a dose optimizer can assign emission times for those beamlets to optimize the dose distribution. However, as the quality of a dose distribution can be evaluated based on dose-volume metrics, such as the D₉₀: minimum dose that covers 90% of the high risk clinical tumor volume; and the D_{2cc}: minimum dose that is absorbed in the most irradiated 2 cm³ of each individual organ at risk, and these metrics can be non-convex. Due to the non-convex nature of these metrics, optimizing the dose distribution regarding the emission times can be time consuming. In one aspect, instead of using [symbol] (n²) possible beamlets, the optimization can be accomplished with a set of n beamlets with a fine azimuthal emission angle φ which are called baseline beamlets. Dose optimization with baseline beamlets can yield high-quality dose distributions, but the delivery is typically impractical as it can require a long time to finish. The output of dose optimization, in one aspect, defines an integral function t assigning each baseline beamlet an integral emission time. The delivery time can be the total emission time of all n baseline beamlets, which can be impractical from a clinical standpoint. RSBT can be time-critical since the process should occur rapidly in order to ensure effective utilization of clinical resources, as the patient is typically under general, epidural, or spinal anesthesia throughout the process. It follows that an additional sequencing step configured to make a trade-off between the delivery time and the dose quality could be beneficial. In one aspect, to reduce the delivery time, several consecutive baseline beamlets can be combined into a larger deliverable beamlet Bₖ, denoted by <abₖ,bₖ,hₖ> with the leading field edge pointing to αₖ=aₖφ and the tailing field edge pointing to βₖ=bₖφ with an emission time hₖ. The delivery time can then be the total sum of hₖ of all those deliverable beamlets used. Given a delivery time threshold H, the sequencing problem can be to find a set B of deliverable beamlets whose total delivery time is no larger than H and well approximates the dose distribution output by the dose optimization with minimum dose errors, that is, Σ_{χ=0}ⁿ⁻¹( [symbol] _{B}(χ)−t(χ))² is minimized. In another aspect, due to the physical constraint of the shielding device illustrated in FIG. 20, there is a maximum opening w of the deliverable beamlets. Hence, the D-RSBT sequencing problem can be modeled as a CIBD problem.

It should be appreciated that the CIBD problem arises from D-RSBT. In certain scenarios, the CIBD problem can have similarities to the Generalized Shape Rectangularization (GSR) problems and the Coupled Path Planning (CPP) problems for Intensity-Modulated Radiation Therapy. In one aspect, one significant difference between the CIBD problem and GSR/CPP is that the CIBD problem is defined on a circular interval with the maximum window constraint; whereas the GSR/CPP problem is defined on a linear interval. The circularity of the problem domain and the maximum window constraint introduce complexity into the CIBD problem. In another aspect, the CIBD problem is also closely related to the DCCF₀ problem, in which the energy function ε₀(y)=Σ_{(u,v)εE}Vᵤᵥ(χᵥ−χᵤ) is minimized subject to y ε [symbol] ^{V}, where Vᵤᵥ are convex functions. DCC [symbol] ₀ can be solved by the algorithm proposed by Ahuja, et al. (Management Science 49(7), 950964 (2003)) with time [symbol] (nm log(n²/m) log(nK)), which is the best known algorithm on this problem. Other differences between the CIBD problem and the DCC [symbol] ₀ problem include: (i) CIBD is not L [symbol] -convex due to the maximal window constraints and the circular domain constraint; and (ii) the number of functions Vᵤᵥ is bounded by [symbol] (n).

In one aspect, the challenges arising from the maximal window constraint and the circularity of the CIBD problem when formulated as a convex cost integer dual network flow problem can be dealt with to enable a solution to the CIBD problem in [symbol] (n² log nH) time. Due to the space limit, the details on the proofs of lemmas, theorems and the algorithmic details are found in the Appendix A.

In one aspect, the CIBD problem can be defined on a circular interval C=[0, n−1], and a window function (a block) can be defined on a sub-interval [a_{b}, bₖ]⊂C with aₖ, bₖε[0, n−1]. Without loss of generality, a block is a feasible if and only if [symbol] (aₖ, bₖ, hₖ) with bₖ>aₖ≧0, (bₖ−aₖ)≦w, aₖ<n and hₖ>0 [symbol] . Thus, aₖε[0, n−1) and bₖε[0, n+w−1].

Definition 1. A blockset B is feasible if and only if ∀Bₖ= [symbol] aₖ, bₖ, hₖ [symbol] εB, aₖε[0, n−1], bₖε[0,n+w−1], 1≦bₖ−aₖ≦w and hₖ>0.

Definition 2. Two blockset B and B′ are equivalent if and only if [symbol] _{B}= [symbol] _{B′} and H_{B}=H_{B′}, where [symbol] _{B}= [symbol] _{B′} stands for a function equivalence: ∀χε[0,n−1], [symbol] _{B}(χ)= [symbol] _{B′}(χ); and H_{B}=Σₖhₖ stands for the total height of blocks in a blockset B.

Definition 3. A feasible blockset B={ [symbol] aₖ, bₖ, hₖ [symbol] |kε[1, K]} is canonical if and only if B satisfies the following properties:

CB1. ∀kε[1,K−1], aₖ≦aₖ₊₁, bₖ≦bₖ₊₁;

CB2. b_{K}−n≦b₁;

Lemma 1. For any feasible blockset B, there exists a canonical blockset B̅={ [symbol] āₖ, b̅ₖ, h̅ₖ [symbol] |kε[1, K̅]} such that B̅ and B are equivalent.

According to Lemma 1, the CIBD problem can be solved by considering canonical blocksets only.

In one aspect, a pair of functions ( [symbol] , [symbol] ) for a canonical blockset

$$B = \{ {B}_{1} , {B}_{2} , … , {B}_{K} \} , can be defined as follows . \\ ℒ ( x ) = {\sum }_{{B}_{k} ∈ B , {a}_{k} \leq x} {h}_{k} , ∀ x ∈ [ 0 , n - 1 ]$$

$$ℛ ( x ) = {\sum }_{{B}_{k} ∈ B , {b}_{k} \leq x} {h}_{k} , ∀ x ∈ [ 0 , n + w - 1 ]$$

$${ℱ}_{( ℒ , ℛ )} ( x ) = \{ \begin{matrix}ℒ ( x ) - ℛ ( x ) + ℒ ( n - 1 ) - ℛ ( n + x ) , ∀ x < w \\ ℒ ( x ) - ℛ ( x ) , ∀ x \geq w\end{matrix}$$

Notice that [symbol] (n−1)=Σ_{k,a}_{ₖ}_{≦n-1}hₖ=Σₖ₌₁^{K}hₖ=H_{B}.

Lemma 2. If ( [symbol] , [symbol] ) is defined with a canonical blockset B, then [symbol] ₍ [symbol] _{,} [symbol] ₎(χ)= [symbol] _{B}(χ) for any χε[0, n+w−1].

Definition 4. A function pair ( [symbol] , [symbol] ) with [symbol] :[0, n−1]→ [symbol] and [symbol] :[0, n+w−1]→ [symbol] is admissible if and only if ( [symbol] , [symbol] ) satisfies the following properties;
AD1: [symbol] and [symbol] are non-negative, [symbol] (0)=0;
AD2: [symbol] and [symbol] are monotonically non-decreasing, i.e. ∀χε[0, n−2], [symbol] (χ)≦ [symbol] (χ+1); ∀χε[0, n+w−2], [symbol] (χ)≦ [symbol] (χ+1);
AD3: ∀χε[0, n−1], [symbol] (χ)≧ [symbol] (χ); ∀χε[n,n+w−1], [symbol] (n−1)≧ [symbol] (χ); particularly, [symbol] (n+w−1)= [symbol] (n−1);
AD4: ∀χε[0, n−1], [symbol] (χ)≦ [symbol] (χ+w);
AD5: ∀χε[0, n−1], [symbol] (χ)≧ [symbol] (χ+1);
AD6: ∀χ≧b₁+n, [symbol] (χ)= [symbol] (n−1), where b₁=min arg( [symbol] (χ)>0).

Lemma 3. If a function pair ( [symbol] , [symbol] ) is defined with a canonical blockset B, then it is admissible.

Lemma 4. If a function pair ( [symbol] , [symbol] ) is admissible, then there exists a canonical blockset B, with [symbol] _{B}(χ)= [symbol] ₍ [symbol] _{,} [symbol] ₎(χ) for any χε[0, n+w−1], and [symbol] (n−1)=H_{B}.

Theorem 1. For any canonical blockset B, we can find an admissible function pair ( [symbol] , [symbol] ) with [symbol] _{B}(χ)= [symbol] ₍ [symbol] _{,} [symbol] ₎(χ), H_{B}= [symbol] (n−1),and vice versa.

In one aspect, according to Theorem 1, the objective of the CIBD problem can be formulated as:

$$min ɛ ( ℒ , ℛ ) = {\sum }_{x = 0}^{w - 1} {( ℒ ( x ) - ℛ ( x ) + ℒ ( n - 1 ) - ℛ ( n + x ) - f ( x ) )}^{2} + {\sum }_{x = w}^{n - 1} {( ℒ ( x ) - ℛ ( x ) - f ( x ) )}^{2} \qquad Eq . ( 11 )$$

However, not all properties can be expressed with linear constraints defined with ( [symbol] , [symbol] ) since b₁ in (AD6) remains unknown until ( [symbol] (χ), [symbol] (χ)) is known. In one aspect, moreover, Equation (6) is not sub-modular since the off-diagonal non-positivity cannot stand with more than 2 variables in a single term of the quadratic objective function, and lacking of sub-modularity can make this problem hard to solve.

In one aspect, the following transformation for admissible function pairs ( [symbol] , [symbol] ): can be introduced.

$$\overline{ℛ} ( x ) = \{ \begin{matrix}ℛ ( n + x ) - ℒ ( n - 1 ) , & ∀ x ∈ [ 0 , {b}_{1} - 1 ] \\ ℛ ( x ) , & ∀ x ∈ [ {b}_{1} , n - 1 ]\end{matrix} \qquad Eq . ( 12 )$$

In a further aspect, the CIBD problem can then be formulated, as follows.

$$\begin{matrix}min ɛ ( ℒ , \overline{ℛ} ) = {\sum }_{x = 0}^{n - 1} {( ℒ ( x ) - \overline{ℛ} ( x ) - f ( x ) )}^{2} & Eq . ( 13 ) \\ s . t . ℒ ( x ) \leq ℒ ( x + 1 ) , ∀ x ∈ [ 0 , n - 2 ] & ( 13 a ) \\ \overline{ℛ} ( x ) \leq \overline{ℛ} ( x + 1 ) , ∀ x ∈ [ 0 , n - 2 ] & ( 13 b ) \\ ℒ ( x ) \leq \overline{ℛ} ( x + w ) , ∀ x ∈ [ 0 , n - w - 1 ] & ( 13 c ) \\ ℒ ( x ) \leq \overline{ℛ} ( x + w - n ) + ℒ ( n - 1 ) , ∀ x ∈ [ n - w , n - 1 ] & ( 13 d ) \\ \overline{ℛ} ( x ) \leq ℒ ( x - 1 ) , ∀ x ∈ [ 1 , n - 1 ] & ( 13 e )\end{matrix}$$

(13f)

Lemma 5. For any admissible function pair ( [symbol] , [symbol] ), ( [symbol] , [symbol] ) is feasible to Equation (8) with ε( [symbol] , [symbol] )=ε( [symbol] , [symbol] ); and for any feasible solution ( [symbol] , [symbol] ) to Equation (13), there exist an admissible function pair ( [symbol] , [symbol] ) such that ε( [symbol] , [symbol] )=ε( [symbol] , [symbol] ).

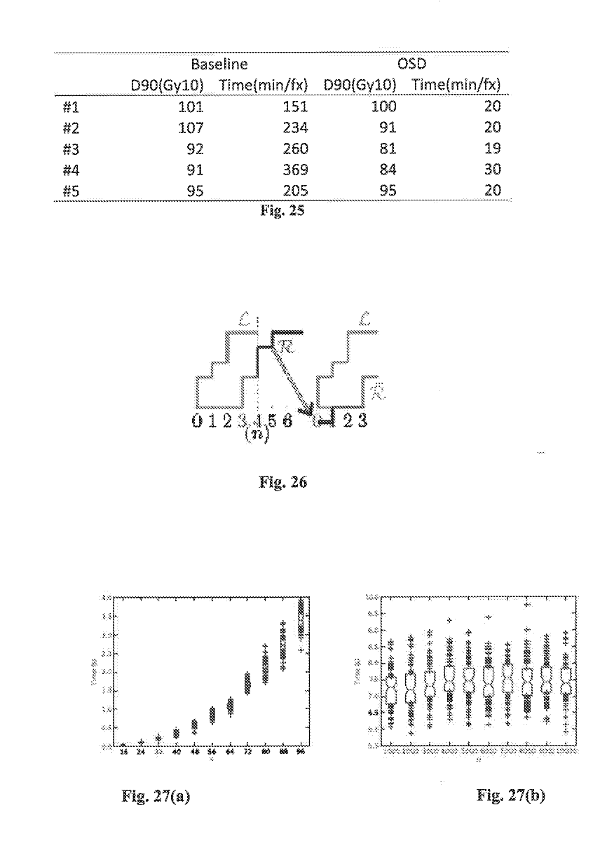

Proof. As illustrated in FIG. 26, to illustrate the correctness of Lemma 5, first, the one-to-one correspondence between admissible function pairs ( [symbol] , [symbol] ) and feasible solutions ( [symbol] , [symbol] ) to Equation (12) can be shown.

According to Equation (12), in one aspect, any admissible function pairs ( [symbol] , [symbol] ) can be uniquely mapped to a solution ( [symbol] , [symbol] ) by shifting [symbol] (χε[n, n+w−1])ₙ units leftwards and [symbol] (n−1) units downwards, as illustrated by FIG. 26. Notice that, the domain of [symbol] is reduced from [0, n+w−1] to [0, n−1] compared to R as the shifting operation overlapped the intervals [0, w−1] and [n, n+w−1]. According to (AD6), ∀χε[0, b₁], [symbol] (χ)=0, and ∀χε[b₁, w−1], [symbol] (n+χ)= [symbol] (n−1); i.e. ∀χε[0, w−1], either [symbol] (χ)=0 or [symbol] (n+χ)= [symbol] (n−1) or both. That can serve to enable a unique mapping from ( [symbol] , [symbol] ) back to ( [symbol] , [symbol] ) with, in one example, the following equation:

$$\overline{ℛ} ( x ) = \{ \begin{matrix}\overline{ℛ} ( x ) , x ∈ [ w , n - 1 ] or ( x < w , \overline{ℛ} ( x ) \geq 0 ) \\ 0 , x < w , \overline{ℛ} ( x ) < 0 \\ ℒ ( n - 1 ) , x \geq n , \overline{ℛ} ( x - n ) \geq 0 \\ ℒ ( n - 1 ) + \overline{ℛ} ( x - n ) , x \geq n , \overline{ℛ} ( x - n ) < 0\end{matrix} \qquad Eq . ( 14 )$$

Together with Equation (14), Equations (13a) and (13b) can be used to enforce the non-decreasing property AD2; Equations (13c) and (13d) can enforce the maximal window constraint AD4; Equation (13e) can encode AD5 which can exclude infeasible blocks with 0 width; AD6 can be enforced by [symbol] (0)≦0 based on Equations (12) and (14); the non-negativity AD1 can be inferred from [symbol] (0)≧0. Equations (13a) and (13b); and [symbol] (n−1)≦H can be used to enforce the constraint on total height of blocks. AD3 is inferred by AD2 and AD5.

In one aspect, the optimization problem in Equation (13) can be solved in [symbol] (n² log(nH)) time with Ahuja's algorithm f [symbol] (n−1) is known. Thus, it can be solved in [symbol] (n²H log(nH)) time. In another aspect, discovering the following property of the problem enables an improved method for optimization.

Theorem 2. If there exist some feasible solution to Equation (13), i.e. dom ε≠, and H≦Σ_{χ=0}ⁿ⁻¹ƒ(χ), then there exist a solution y*=( [symbol] *, [symbol] ) such that [symbol] *(n−1)=H and ∀yεdom ε, ε(y*)≦ε(y).

Proof. Theorem 2 can be proved in a constructive way, i.e. suppose there exists some other optimal solution y′=( [symbol] ′, [symbol] ′) such that

H′= [symbol] ′(n−1)≦H and ∀yεdom ε, ε(y′)≦ε(y), then another solution can be y*=( [symbol] *, [symbol] *) with [symbol] *(n−1)=H and ε(y*)≦ε(y′).

The construction of y* differs in two different cases. For the first case, if H≦Σ_{χ=0}ⁿ⁻¹( [symbol] ′(χ)− [symbol] ′(χ)), set y*=( [symbol] ′+δ, [symbol] ′+δ), where

$$δ ( x ) = \{ \begin{matrix}min \{ - {\overline{ℛ}}^{′} ( 0 ) , H - {H}^{′} \} & x = 0 \\ min \{ {ℒ}^{′} ( x - 1 ) - {\overline{ℛ}}^{′} ( x ) + δ ( x - 1 ) , H - {H}^{′} \} , & x > 0\end{matrix} \qquad Eq . ( 15 )$$

In one aspect, the function δ can be applied to y′ in order to make the new solution y*=( [symbol] *, [symbol] *) can satisfy [symbol] *(n−1)=H without changing the objective value while preserving all the constraints.

For the second case, where

H>Σ_{χ=0}ⁿ⁻¹( [symbol] ′(χ)− [symbol] ′(χ)), let y″=( [symbol] ″, [symbol] ″)=( [symbol] ′+δ, [symbol] ′+δ), where δ can be the same as defined in Equation (15). As same as the previous case y″ can be feasible to Equation (13) and ε(y″)=ε(y′), however, [symbol] ″(n−1)<H. But, in one aspect, y″ has its specialties: [symbol] ″(0)=0 and ∀χε[0, n−1], [symbol] ″(χ)= [symbol] ″(χ−1) (define [symbol] ″(−1)=0). By enforcing these two specialties into Equation (13), Equation (13b)-(13e) becomes redundant, and Equation (13f) can be rewritten to [symbol] (n−1)≦H, [symbol] (0)=0. In one aspect, the constraint [symbol] (0)=0 as CIBD″. can be relaxed. Assuming [symbol] °(n−1)=H, finding the solution y°= [symbol] ° with ε″( [symbol] °)=0 to CIBD″ can be done in linear time (the objective function of CIBD″ is defined as ε″( [symbol] )=Σ_{χ=0}ⁿ⁻¹( [symbol] (χ)− [symbol] (χ−1)−ƒ(χ))², the [symbol] part of the solution is omitted since it can be determined by [symbol] ).

Then, y*=( [symbol] *, [symbol] *) can be assigned with ∀χε[0,n−1], [symbol] *(χ)=( [symbol] ″ [symbol] [symbol] °)(χ), [symbol] *(χ)=( [symbol] ″ [symbol] [symbol] °)(χ−1). According to the L [symbol] -convexity of CIBD″, ε(y*)=ε′( [symbol] ″ [symbol] [symbol] °)≦ε′( [symbol] ″)=ε(y′). By further showing ( [symbol] ″ [symbol] [symbol] °)(−1)= [symbol] ″(−1)=0, ( [symbol] ″ [symbol] [symbol] °)(n−1)= [symbol] °(n−1)=H, it can be shown that y* is feasible to Equation (13) and it can also be a global optimizer.

According to Theorem 2, whenever H≦Σ_{χ=0}ⁿ⁻¹ƒ(χ), Equation (13) can be solved by setting [symbol] (n−1)=H. Setting [symbol] (n−1)=H makes Equation (13) a convex cost integer dual network flow problem, which can be solved in time [symbol] (n² log(nH)) for this case. In another aspect, if H>Σ_{χ=0}ⁿ⁻¹ƒ(χ), it can be solved in linear time.

Although Ahuja's algorithm has the best know theoretical complexity, Kolmogorov et al. (Mathematical Programming (2007)) found that their algorithm runs better in practice. We implemented our CIBD algorithm using C++ base on Kolmogorov's framework with a specialized local search step (see Appendix B) and the total time complexity is [symbol] (n³ log n log H). In one example, for the combinations of parameters n and H, 100 computer-generated exemplary cases were used to test the efficiency of the disclosed methods for optimizing treatment delivery of D-RSBT.

FIG. 27(a) and FIG. 27(b) show the impact parameters n and H can have on the running time, respectively. In one aspect, based on exemplary results, the running time can quadratically increase with n but is not noticeably impacted by H.

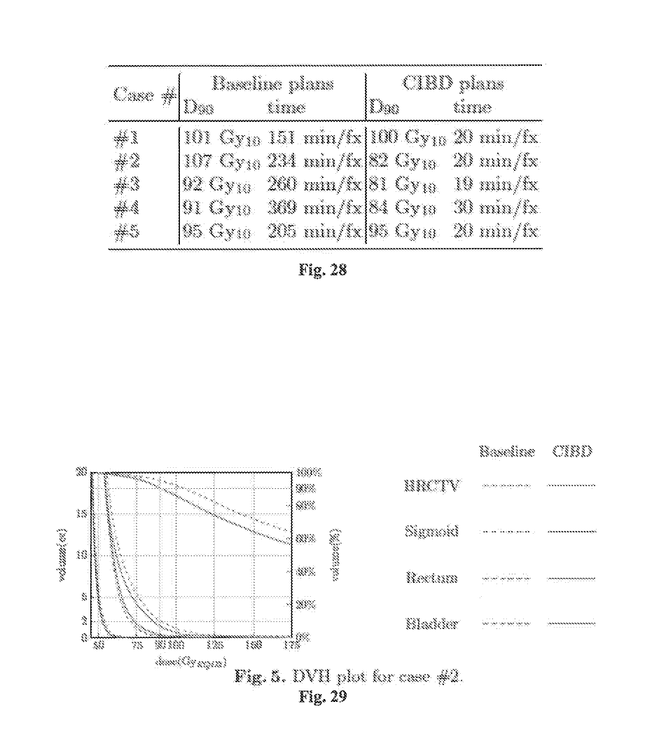

In another example, the disclosed methods for optimizing treatment delivery of D-RSBT were applied to 5 distinct clinical cases. One example of a DVH (Dose-Volume Histogram) plot for one of the 5 cases is shown in FIG. 29. In a DVH plot, each point on the curve represents the volume of structure (y-axis) receiving greater than or equal to that dose (x-axis). The delivery plans were evaluated with HR-CTV (High Risk Clinical Tumor Volume) D₉₀ and the delivery time (minutes per fraction). In one aspect, all of the clinical cases completed optimization with the disclosed methods within about 1 second. The plan quality comparisons are shown in FIG. 28.

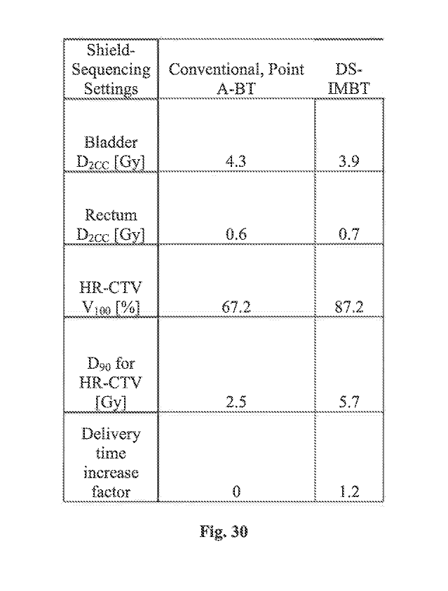

In another example, a partially-shielded electronic BT source with an azimuthally-adjustable shield aperture was employed and treatment plans for a bulky cervical cancer tumor (>40cc) were generated using the present methods. In shielding-sequencing optimization, a non-trivial network transformation scheme was utilized to efficiently find the global optimum with network flow algorithms. The treatment plan goals of MRI-guided, volume-optimization BT per GEC-ESTRO recommendations were utilized. D₉₀ of the HR-CTV receives a prescription dose (Rx) while OAR (rectum & sigmoid (bladder)) D_{2cc}≦75(90) Gy₃ EQD2 (equivalent dose in 2Gy fraction) from external beam radiotherapy and BT. The feasibility was tested using the metric of plan conformality (D₉₀ in HR-CTV and D_{2cc} in OAR) and treatment delivery time. Here, the shield-sequencing algorithm described in this disclosure can improve tumor coverage (D₉₀) with favorable OAR sparing with an acceptable increase in delivery time. The D₉₀ (100% Rx) was improved from 41% Rx from conventional, Point-A plan. D_{2cc} of OAR can be kept under the recommended limits. The increase of delivery time was recorded as less than about 4.2 times higher compared with conventional BT, or less than about 1.2 times higher with D₉₀ improved to 94% Rx. The total optimization time was around 10 minutes. FIG. 30 presents a comparison of the results obtained by conventional BT versus D-RSBT.

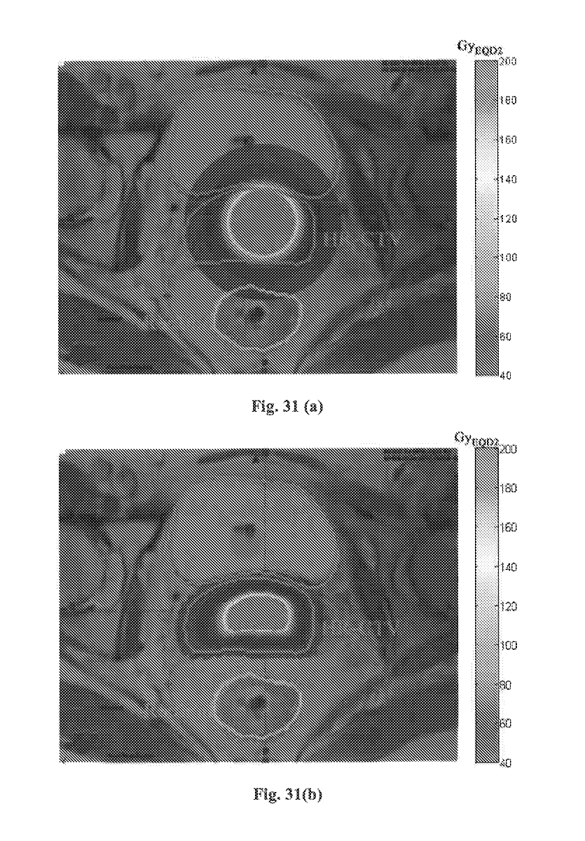

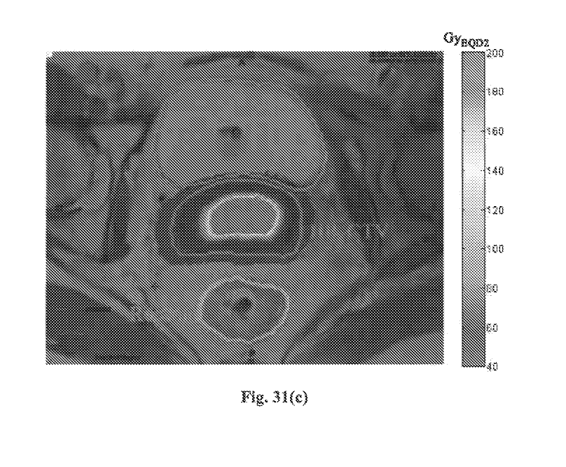

In a further example, FIGS. 31(a)-(c) illustrate treatment planning and results that can be achieved using the disclosed methods for optimizing treatment delivery of D-RSBT. FIG. 31(a) shows a dose distribution for conventional ¹⁹²Ir unshielded source with a D₉₀ of 44.6 Gy₁₀ and a time of 22.4 units. FIG. 31(b) shows a dose distribution for IMBT using the Axxent Xoft eBT source configured with 60 divisions and no overlapping with a D₉₀ of 89.5 Gy₁₀ and a time of 450.6 units. FIG. 31(c) shows a dose distribution for IMBT using the Axxent Xoft eBT source as well as the disclosed methods for optimizing treatment delivery of D-RSBT with a D₉₀ of 82.9 Gy₁₀ and a time of 36.34 units. The conventional approach results in a poor quality while the IMBT approach alone results in a planning time that is not practical. However, using an IMBT in conjunction with the disclosed methods for optimizing treatment delivery, a 38 Gy₁₀ increase over conventional BT and a 6 Gy₁₀ decrease over non-overlapping fine resolution IMBT alone is achieved. Additionally, this is accomplished within only about 1.6 times the conventional ¹⁹²Ir delivery time.

Appendix F illustrates yet another example of dynamic rotating-shield intensity modulated brachytherapy using the combinatorial optimization model for sequencing the rotating shields with dynamic-sized opening described in this disclosure used in the treatment of cervical cancer.

In another aspect, the disclosed methods for optimizing treatment delivery of D-RSBT also provide a means for computing the tradeoff between the delivery time and D₉₀ such that a treatment provider may optimize treatment for a given clinical case by selecting different time budgets or quality goals.

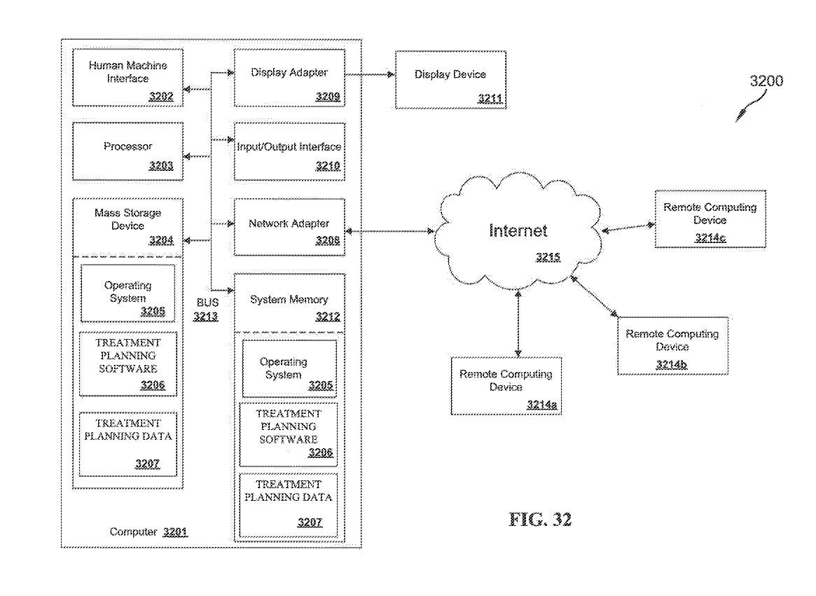

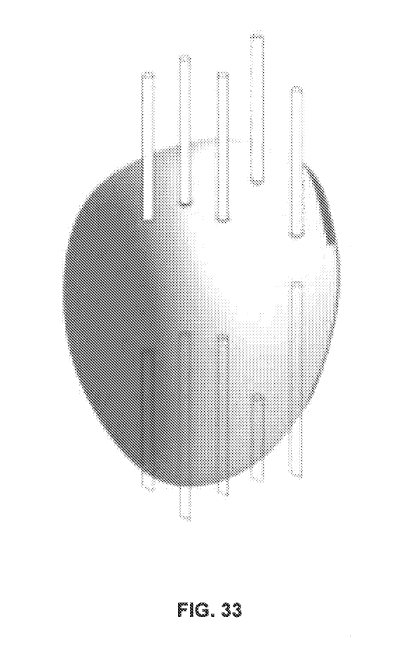

FIG. 32 illustrates a block diagram of an exemplary operating environment 3200 that enables various features of the subject disclosure and performance of the various methods disclosed herein. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The various embodiments of the subject disclosure can be operational with numerous other general purpose or special purpose computing system environments or configurations. Examples of well known computing systems, environments, and/or configurations that can be suitable for use with the systems and methods comprise, but are not limited to, personal computers, server computers, laptop devices or handheld devices, and multiprocessor systems. Additional examples comprise wearable devices, mobile devices, set top boxes, programmable consumer electronics, network PCs, minicomputers, mainframe computers, distributed computing environments that comprise any of the above systems or devices, and the like.

The processing effected in the disclosed systems and methods can be performed by software components. The disclosed systems and methods can be described in the general context of computer-executable instructions, such as program modules, being executed by one or more computers or other computing devices. Generally, program modules comprise computer code, routines, programs, objects, components, data structures, etc. that perform particular tasks or implement particular abstract data types. The disclosed methods also can be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 3201. The components of the computer 3201 can comprise, but are not limited to, one or more processors 3203, or processing units 3203, a system memory 3212, and a system bus 3213 that couples various system components including the processor 3203 to the system memory 3212. In the case of multiple processing units 3203, the system can utilize parallel computing.

In general, a processor 3203 or a processing unit 3203 refers to any computing processing unit or processing device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally or alternatively, a processor 3203 or processing unit 3203 can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors or processing units referred to herein can exploit nano-scale architectures such as, molecular and quantum-dot based transistors, switches and gates, in order to optimize space usage or enhance performance of the computing devices that can implement the various aspects of the subject disclosure. Processor 3203 or processing unit 3203 also can be implemented as a combination of computing processing units.

The system bus 3213 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 3213, and all buses specified in this description also can be implemented over a wired or wireless network connection and each of the subsystems, including the processor 3203, a mass storage device 3204, an operating system 3205, treatment planning software 3206, treatment planning data 3207, a network adapter 3208, system memory 3212, an Input/Output Interface 3210, a display adapter 3209, a display device 3211, and a human machine interface 3202, can be contained within one or more remote computing devices 3214a,b,c at physically separate locations, connected through buses of this form, in effect implementing a fully distributed system.

In one aspect, treatment planning software 3206 can comprise computer-executable instructions for implementing the various methods described herein, such as exemplary method 2300. In another aspect, treatment planning software 3206 can include software to control various aspects of manufacturing of the radiation shield and, as part of manufacturing, treating a surface in accordance with aspects described herein in order to attain a desired thickness profile for the surface of the radiation shield. In certain embodiments, treatment planning software 3206 also can include computer-executable instruction for selecting radio-opaque materials for manufacturing the radiation shield. Treatment planning software 3206 and treatment planning data 3207 configure processor 3203 to perform the one or more steps of the methods described herein. In addition or in the alternative, treatment planning software 3206 and treatment planning data 3207 can configure processor 3203 to operate in accordance with various aspects of the subject disclosure.

The computer 3201 typically comprises a variety of computer readable media. Exemplary readable media can be any available media that is accessible by the computer 3201 and comprises, for example and not meant to be limiting, both volatile and non-volatile media, removable and non-removable media. The system memory 3212 comprises computer readable media in the form of volatile memory, such as random access memory (RAM), and/or non-volatile memory, such as read only memory (ROM). The system memory 3212 typically contains data and/or program modules such as operating system 3205 and treatment planning software 3206 that are immediately accessible to and/or are presently operated on by the processing unit 3203. Operating system 2405 can comprise OSs such as Windows operating system, Unix, Linux, Symbian, Android, iOS, Chromium, and substantially any operating system for wireless computing devices or tethered computing devices.

In another aspect, the computer 3201 also can comprise other removable/non-removable, volatile/non-volatile computer storage media. By way of example, FIG. 32 illustrates a mass storage device 3204 which can provide non-volatile storage of computer code, computer readable instructions, data structures, program modules, and other data for the computer 3201. For example and not meant to be limiting, a mass storage device 3204 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 3204, including by way of example, an operating system 3205, and treatment planning software 3206. Each of the operating system 3205 and treatment planning software 3206 (or some combination thereof) can comprise elements of the programming and the treatment planning software 3206. Data and code (e.g., computer-executable instruction(s)) can be retained as part of treatment planning software 3206 and can be stored on the mass storage device 3204. Treatment planning software 3206, and related data and code, can be stored in any of one or more databases known in the art. Examples of such databases comprise, DB2®, Microsoft® Access, Microsoft® SQL Server, Oracle®, mySQL, PostgreSQL, and the like. Further examples include membase databases and flat file databases. The databases can be centralized or distributed across multiple systems.

In another aspect, the user can enter commands and information into the computer 3201 via an input device (not shown). Examples of such input devices comprise, but are not limited to, a camera; a keyboard; a pointing device (e.g., a “mouse”); a microphone; a joystick; a scanner (e.g., barcode scanner); a reader device such as a radiofrequency identification (RFID) readers or magnetic stripe readers; gesture-based input devices such as tactile input devices (e.g., touch screens, gloves and other body coverings or wearable devices), speech recognition devices, or natural interfaces; and the like. These and other input devices can be connected to the processing unit 3203 via a human machine interface 3202 that is coupled to the system bus 3213, but can be connected by other interface and bus structures, such as a parallel port, game port, an IEEE 1394 Port (also known as a Firewire port), a serial port, or a universal serial bus (USB).

In yet another aspect, a display device 3211 also can be connected to the system bus 3213 via an interface, such as a display adapter 3209. It is contemplated that the computer 3201 can have more than one display adapter 3209 and the computer 3201 can have more than one display device 3211. For example, a display device can be a monitor, an LCD (Liquid Crystal Display), or a projector. In addition to the display device 3211, other output peripheral devices can comprise components such as speakers (not shown) and a printer (not shown) which can be connected to the computer 3201 via Input/Output Interface 3210. Any step and/or result of the methods can be output in any form to an output device. Such output can be any form of visual representation, including, but not limited to, textual, graphical, animation, audio, tactile, and the like.

The computer 3201 can operate in a networked environment using logical connections to one or more remote computing devices 3214a,b,c. By way of example, a remote computing device can be a personal computer, portable computer, a mobile telephone, a server, a router, a network computer, a peer device or other common network node, and so on. Logical connections between the computer 3201 and a remote computing device 3214a,b,c can be made via a local area network (LAN) and a general wide area network (WAN). Such network connections can be through a network adapter 3208. A network adapter 3208 can be implemented in both wired and wireless environments. Such networking environments are conventional and commonplace in offices, enterprise-wide computer networks, intranets, and the Internet. Networking environments are referred to as network(s) 3215 and generally can be embodied in wireline networks or wireless networks (e.g., cellular networks, such as Third Generation (3G) and Fourth Generation (4G) cellular networks, facility-based networks (femtocell, picocell, Wi-Fi networks, etc.).

As an illustration, application programs and other executable program components such as the operating system 3205 are illustrated herein as discrete blocks, although it is recognized that such programs and components reside at various times in different storage components of the computing device 3201, and are executed by the data processor(s) of the computer. An implementation of treatment planning software 3206 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer-readable media can comprise “computer storage media,” or “computer-readable storage media,” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In various embodiments, the disclosed systems and methods for CBT can employ artificial intelligence (AI) techniques such as machine learning and iterative learning for identifying patient-specific, treatment-specific shields. Examples of such techniques include, but are not limited to, expert systems, case based reasoning, Bayesian networks, behavior based AI, neural networks, fuzzy systems, evolutionary computation (e.g., genetic algorithms), swarm intelligence (e.g., ant algorithms), and hybrid intelligent systems (e.g., Expert inference rules generated through a neural network or production rules from statistical learning).

As described in greater detail below, the subject disclosure relates to advanced intensity-modulated brachytherapy. In one aspect, the disclosure recognizes and overcomes the issue of treating tumors (such as cervical cancer tumors) that non-radially symmetric cancer tumors due to one or more of the shape of the tumor or the location of an intracavitary applicator utilized for treatment. In one scenario, as described herein, M-RSBT can be used to deliver highly patient specific doses to cervical cancer tumors that are impossible to deliver with conventional BT. In another aspect, the disclosure recognizes and overcomes the issue of the treatment time for S-RSBT increasing nonlinearly as a radiation shield emission angle decreases, which can cause treatment times for S-RSBT to be infeasible to implement into the clinic. In one scenario, M-RSBT utilizes a judicious combination of several different shield emission angles to reduce the treatment time of S-RSBT by a significant factor while duplicating the dose distribution exactly. It should be appreciated that the therapy advantages (such as reduced treatment time) of M-RSBT with respect to other RSBT techniques can offset the complexity associated with changing the size of the radiation shield during delivery.

In one aspect, the fields of application of I-RSBT can comprise radiation oncology and urology. In various scenarios, it is anticipated that I-RSBT can be the treatment of choice for patients with localized prostate cancer who are willing and able to undergo 2-4 general anesthesia sessions for the delivery of the therapy.

In one aspect, I-RSBT provides the opportunity for reduced complications and dose escalation for prostate cancer patients, which we expect to result in improved outcomes with reduced toxicity. Urethral dose in HDR-BT has been shown to be closely associated with the incidence of Grade 2 or higher acute genitourinary (GU) toxicity. We expect I-RSBT will have lower urinary complications than radical prostatectomy (RP), external beam radiotherapy (EBRT), low-dose-rate brachytherapy (LDR-BT), and conventional HDR-BT, and will be the preferable approach to treating localized prostate cancers. It is also expected that I-RSBT can reduce rectal toxicity associated with HDR-BT treatments of prostate cancer.

The shielded needle device and automation of its rotation system and software enable the delivery of radiation dose distributions that are non-radially-symmetric about the needle by rotating the needle inside the tumor while it contains a radiation source. This allows the clinician to tailor the radiation dose delivered to a tumor in a manner that significantly reduces the dose delivered to sensitive normal tissue that is inside or adjacent to the tumor, which is the I-RSBT technique. Multiple shielded needles can be used to deliver I-RSBT, which is an HDR-BT technique that entails rotating a radiation-attenuating shield about a BT source in an optimized fashion. In an aspect, the multiple shielded needles used to deliver I-RSBT can comprise shielded needles with different azimuthal shield emission angles. I-RSBT is a type of intensity modulated brachytherapy (IMBT) technique. While IMBT techniques have been introduced in the literature, practical implementation of I-RSBT with radioisotopes remains largely untapped because radioisotopes for I-RSBT delivery may be difficult to obtain or need further development.

Five-year relative survival rates for the nearly 180,000 patients annually diagnosed with localized prostate cancer in the U.S. are almost 100%, independent of the three most common treatment methods used: radical prostatectomy (RP), external beam radiation therapy (EBRT), low-dose-rate brachytherapy (LDR-BT) with permanent ¹²⁵I or ¹⁰³Pd implants. Treatment decisions for localized prostate cancer thus depend strongly on anticipated morbidity, and convenience for the patient.

EBRT and LDR-BT are associated with greater bowel toxicity than RP, but lower urinary incontinence rates. Urinary obstruction/irritation rates are similar for all three therapies, and sexual dysfunction rates are lowest for LDR-BT. HDR-BT is an increasingly popular option for treating localized prostate cancer as a monotherapy, as a single-fraction boost to EBRT, or as a multi-fraction boost to EBRT. Prostate HDR-BT entails ultrasound, computed tomography (CT), or magnetic resonance (MR) image-guided ¹⁹²Ir-BT using at least 14 plastic catheters. The therapy is typically delivered in 2-4 fractions over 1-2 days, and consensus has not been reached on an HDR-BT fractionation scheme for prostate cancer. HDR-BT deliveries are more geometrically stable than those of EBRT in that they are not influenced by inter- or intra-fraction patient motion. With HDR-BT, all radiation delivered to the prostate is tailored to the shape of the prostate, bladder, rectum, and urethra, on the day of treatment. Thus changes in the shape, size, and location of the prostate due to bladder filling and edema can be accounted for in each delivery session. In EBRT, the urethra is typically not spared and is delivered the same dose as the prostate.

HDR-BT is advantageous over LDR-BT in that no radioactive seeds are implanted, eliminating dosimetric uncertainty due to seed migration. In addition, LDR-BT does not exploit the late-responding characteristics of prostate cancer as HDR-BT does. HDR-BT monotherapy has been shown to decrease toxicity rates relative to LDR-BT for grade 1-3 acute dysuria (36% vs. 67%, p<0.001), acute urinary frequency/urgency (54% vs. 92%, p<0.001), acute rectal pain (6% vs. 20%, p<0.017), chronic urinary frequency (54% vs. 92%, p<0.004), and actuarial impotence at 36 months (16% vs. 45%, p<0.062). As LDR-BT is the localized prostate therapy typically associated with lower toxicity rates than RP and EBRT, the lower toxicity of HDR-BT relative to LDR-BT makes HDR-BT the therapy of choice for localized prostate cancer. Typically, HDR-BT is delivered over a fractionated schedule which can require in-patient hospital visits.

The standard-of-care HDR-BT isotope, ¹⁹²Ir, is suboptimal for I-RSBT of any site in which the needle or catheters used must be 2 mm in diameter or smaller. Data and simulations indicate that I-RSBT can be effective, in certain scenarios, if the shield transmission is 10% or less, and ¹⁹²Ir shielded with a 5 mm of tungsten results in a suboptimal 30% transmission.

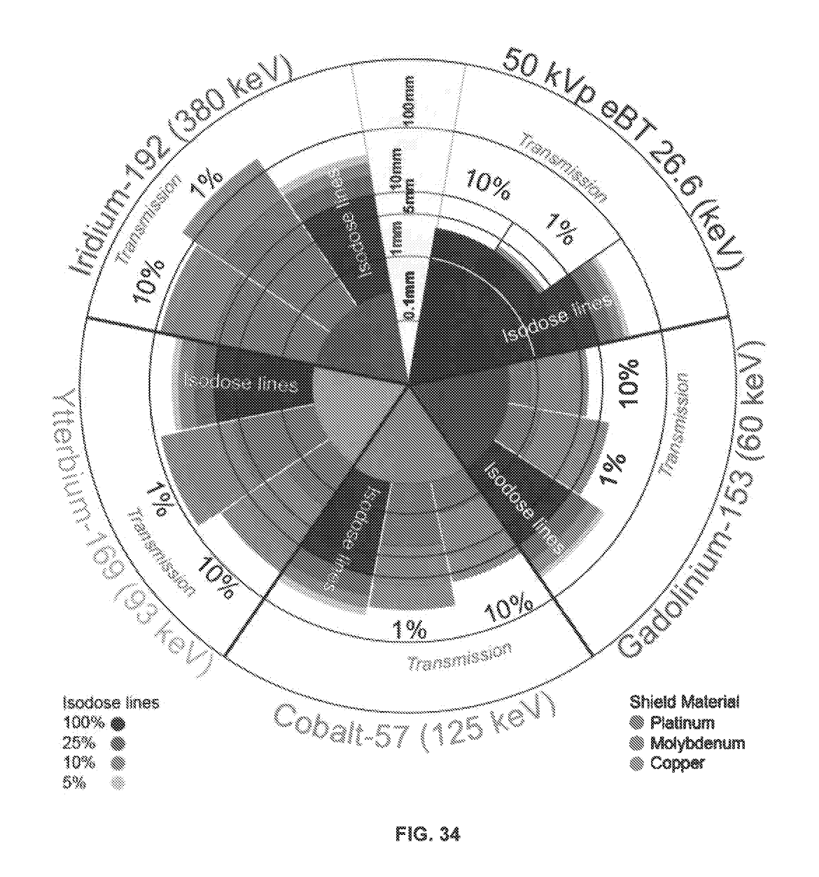

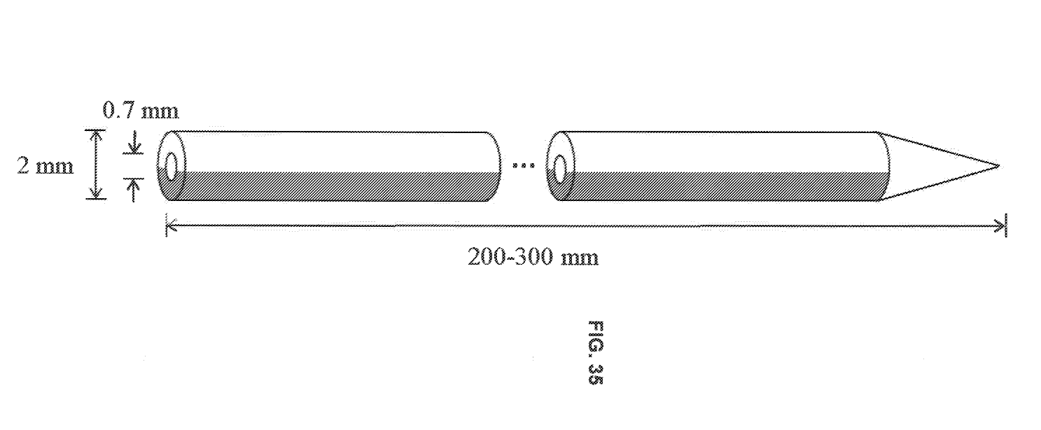

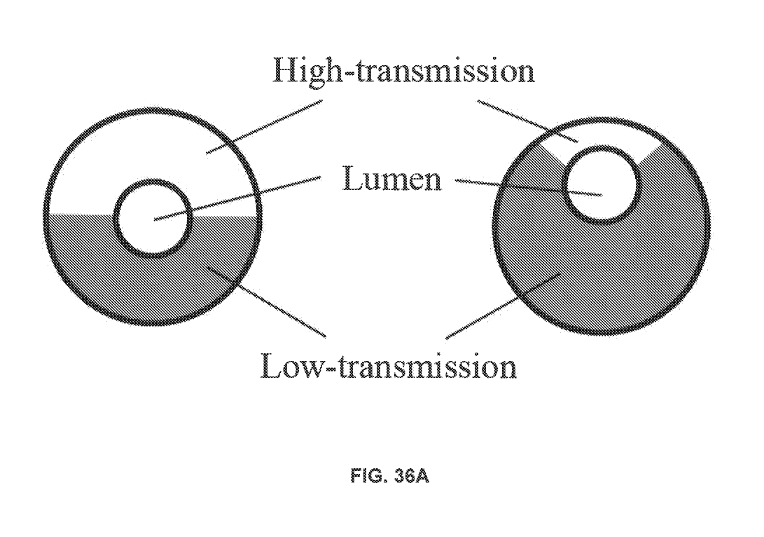

In an aspect, breast and rectal cancers can be treated with ¹⁹²Ir-RSBT using a 1 cm radius tungsten shield. In another aspect, cervical or prostate cancer RSBT can use applicators of less than 5 mm radius and interstitial needles of less than 1 mm radius, respectively. Therefore, the different radiation source/shield combinations for RSBT (FIG. 34) are necessary.

In one aspect, as illustrated in FIG. 34, platinum-shielded ¹⁵³Gd and ⁵⁷Co sources were feasible for interstitial RSBT.

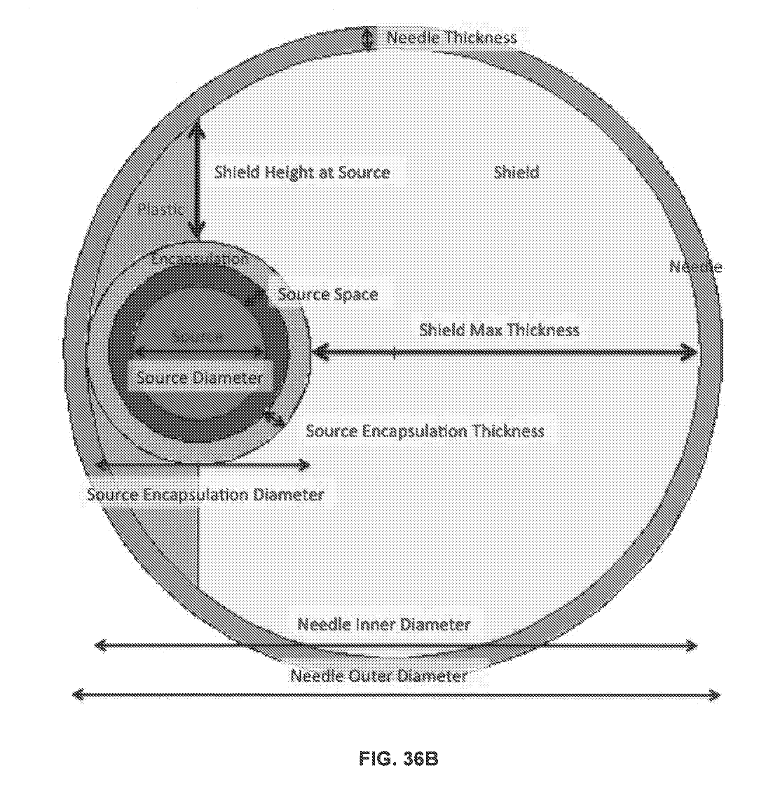

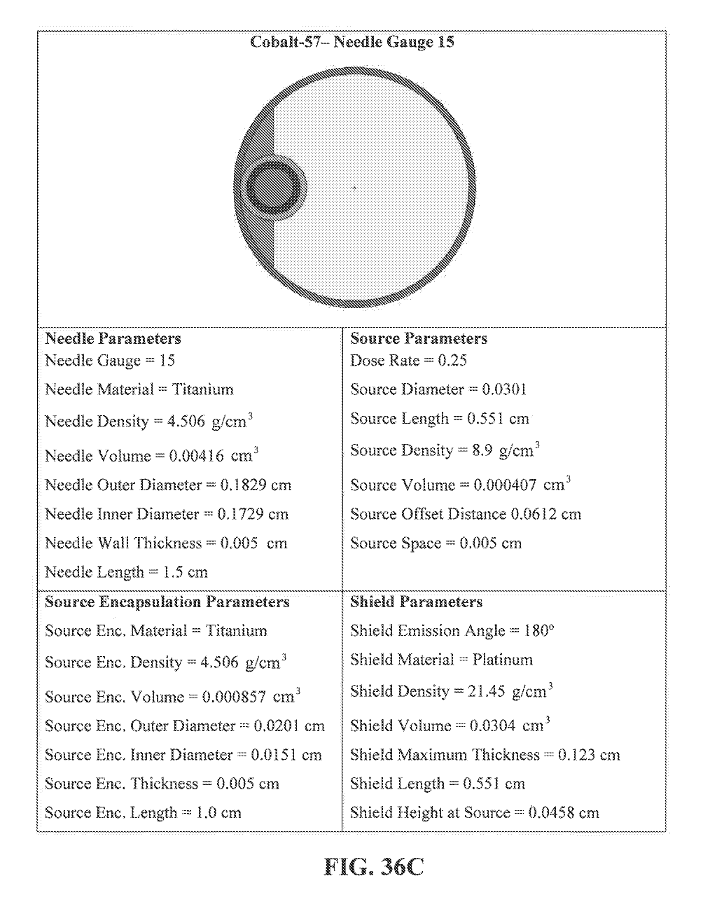

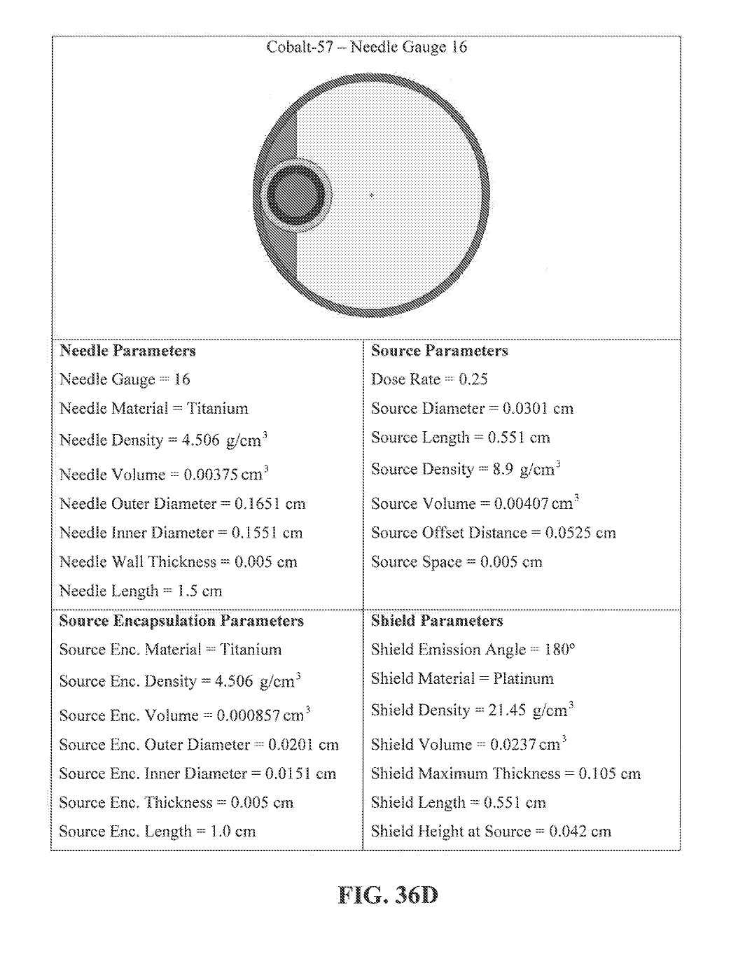

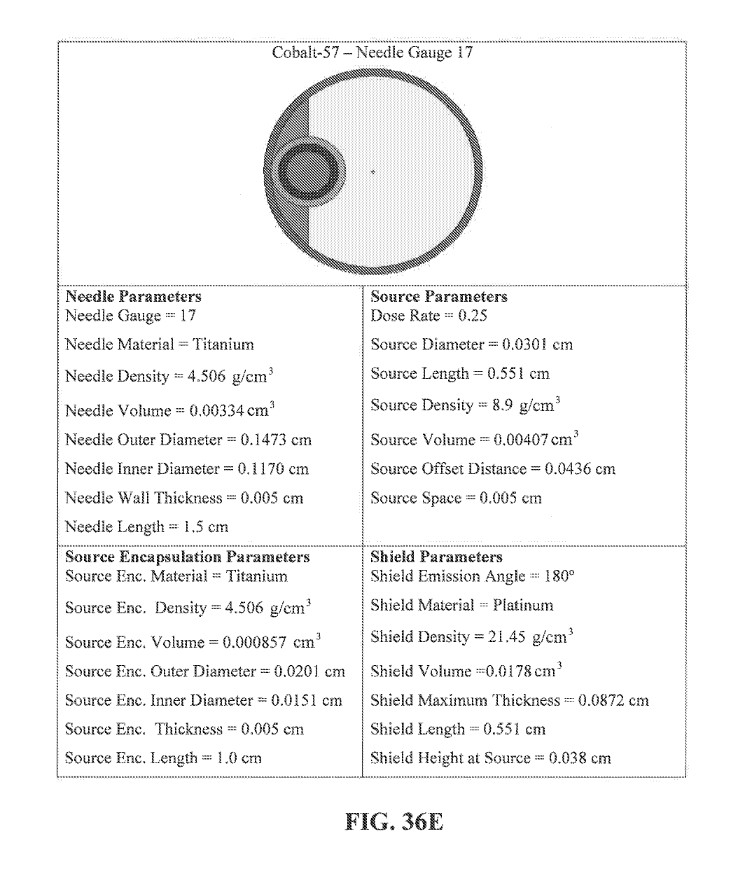

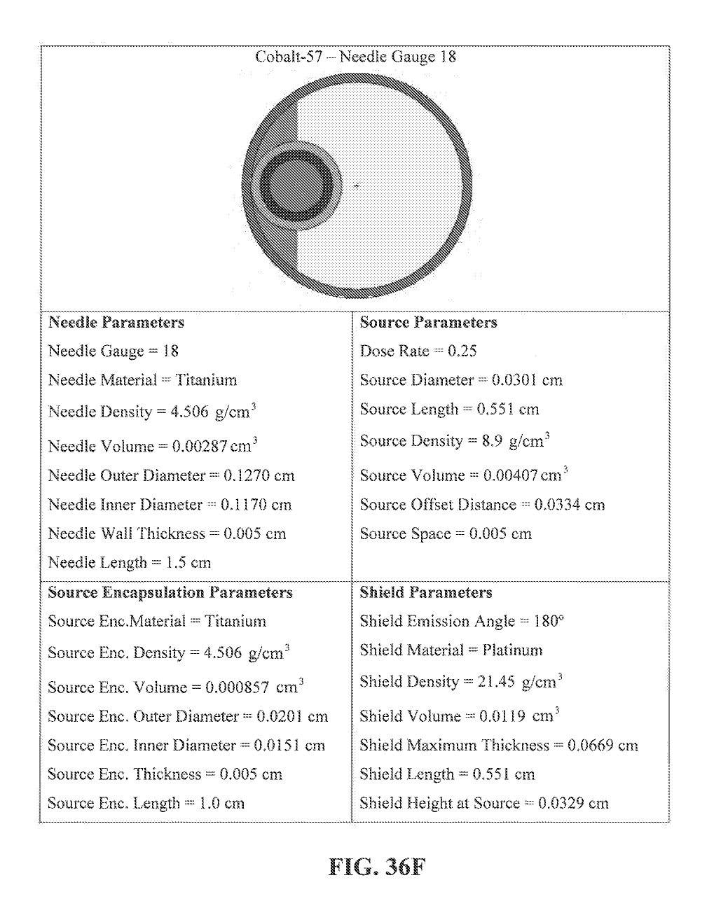

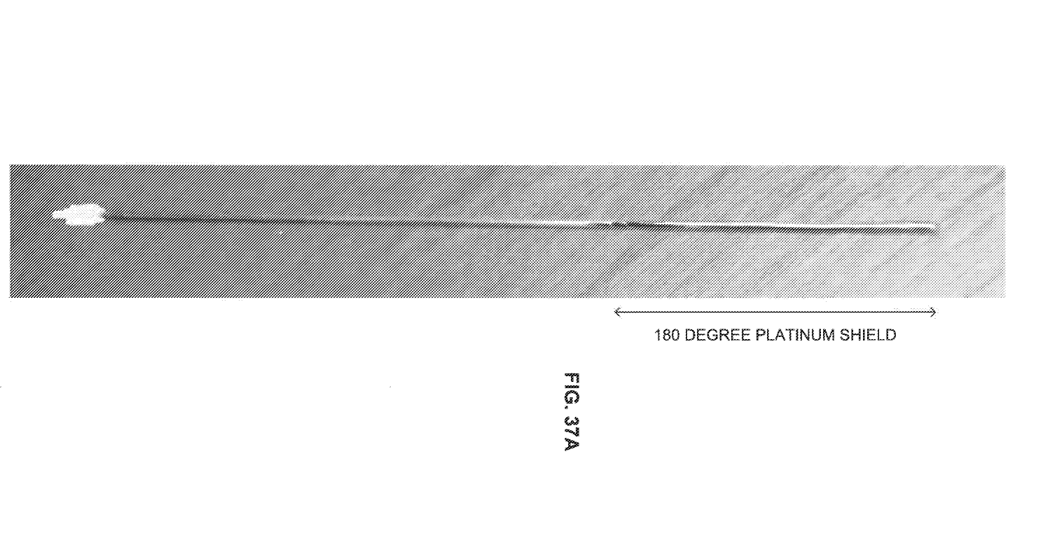

Some aspects provide a catheter for interstitial brachytherapy comprising at least two or more materials that transmit varying quantities of the radiation source used to deliver I-RSBT, and aligned such that their interfaces extend longitudinally along the catheter shaft (see, e.g., FIG. 35). The catheter is encased with a non-toxic material to enable its safe insertion into patient tissue. The materials composing the catheter can extend the entire distance of the catheter shaft, or only along a fraction of the shaft. The catheter lumen can be located at the center of the catheter cross section or off center, as illustrated in FIG. 36A. The emission angles subtended by the materials composing the catheter can vary between 0° and 360°. In certain embodiments, the catheter could also be in the form of a needle with a sharp tip, as shown in FIG. 35.

FIG. 36B illustrates an example cross-section of shaft suitable for application in I-RSBT in accordance with one or more aspects of the subject disclosure. Various embodiments of such shaft can be implemented, as shown in FIGS. 36B-F the color scheme introduced in FIG. 36B indicates the portions of the shaft, and is consistent throughout FIGS. 36B-F)

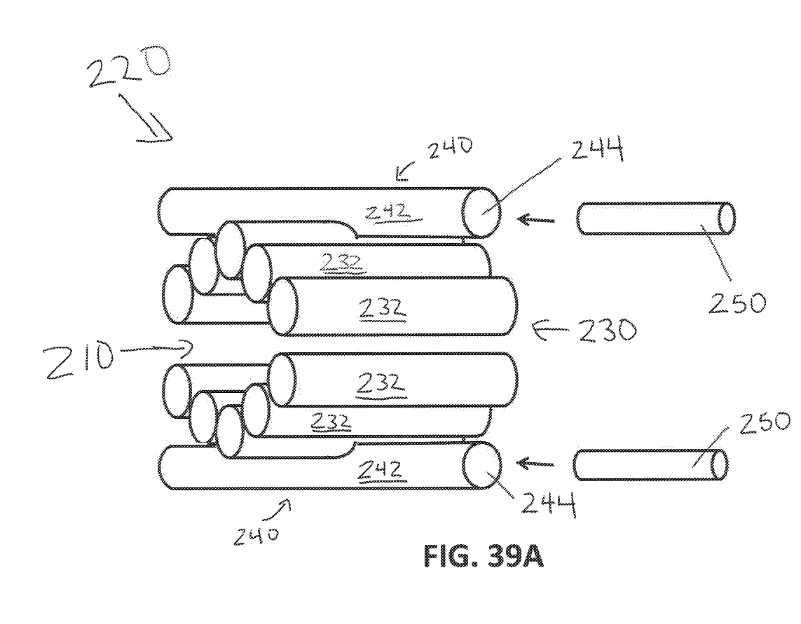

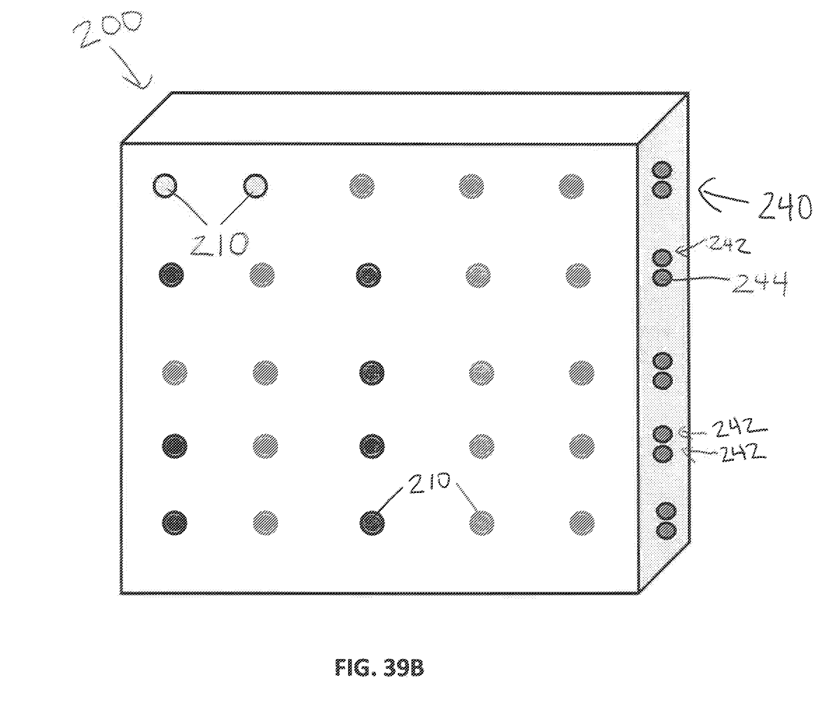

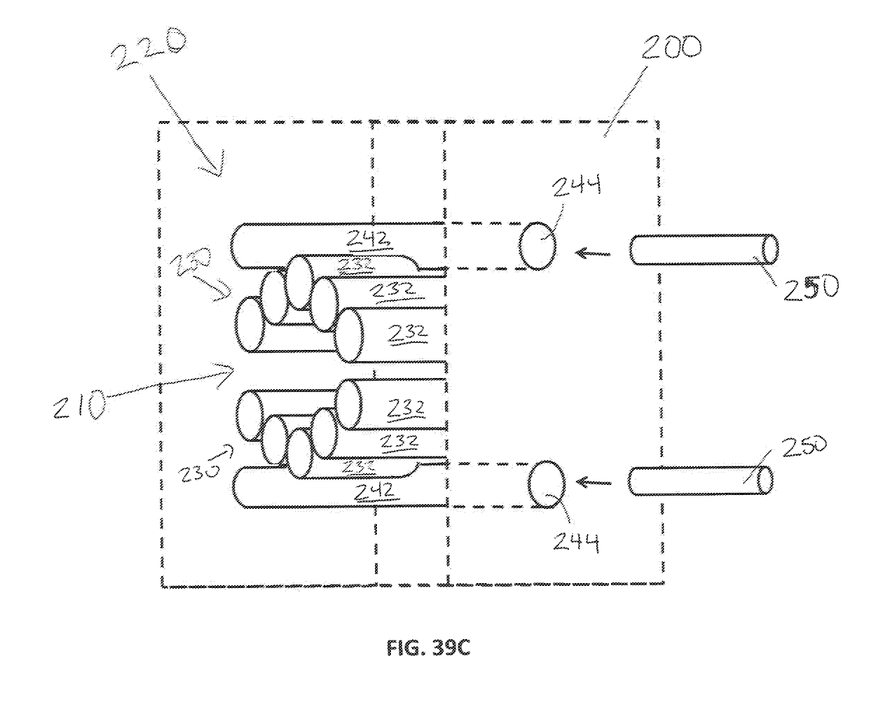

In certain treatment scenarios, I-RSBT can be delivered with a number of different catheters or needles that will inserted into the patient's tissue through a template consisting of a number of holes through which the catheters or needles pass. An example of a template 200 is shown in FIG. 39B. For HDR-BT, delivery templates are often sutured to the patient's perineum during delivery in order to ensure its stability throughout the catheter placement and delivery processes. The I-RSBT template can include an automated mechanism that locks or unlocks a given needle or catheter, enabling independent rotation, discussed in more detail below. The radiation source may be inserted into each needle or catheter individually, and, during delivery, the needle or catheter must be rotated in a manner that is independent of its neighbors.

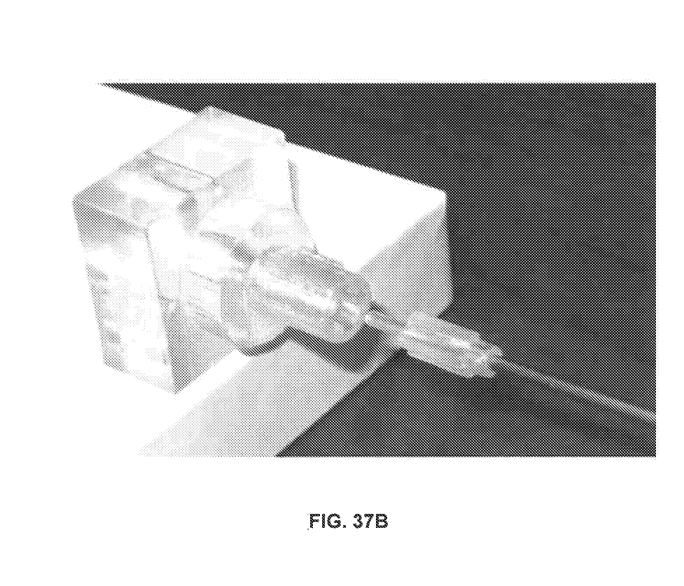

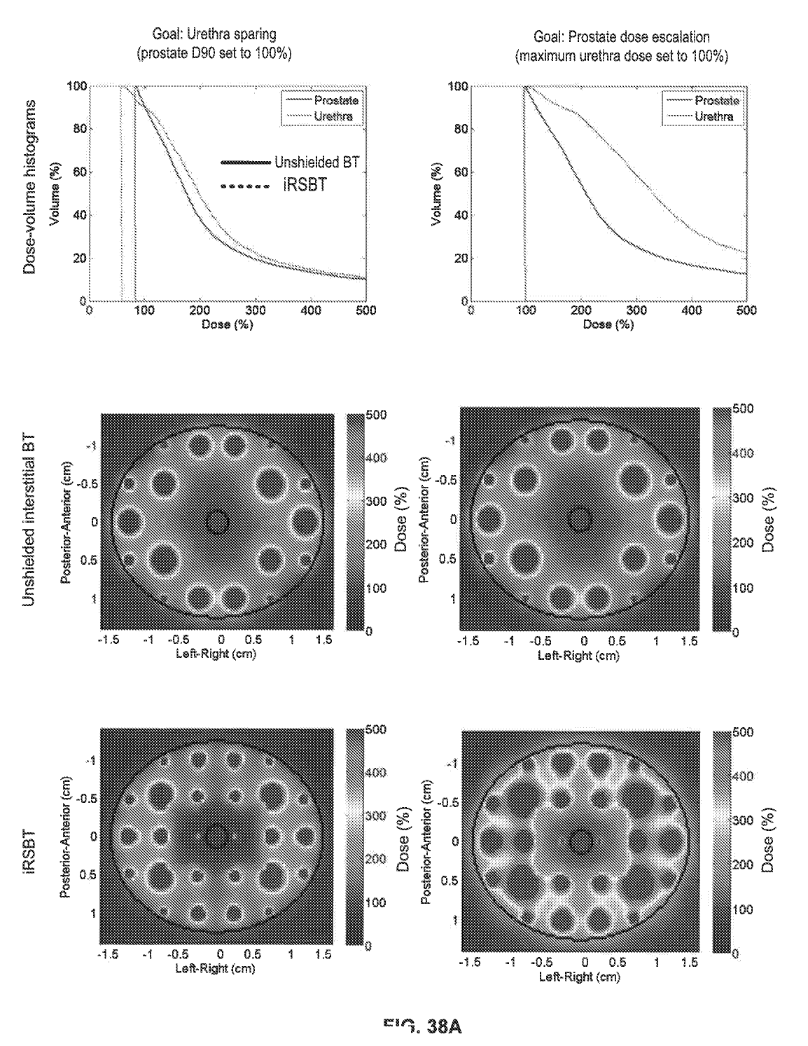

In an aspect, the proximal ends of the shaft (embodying or comprising a catheter and/or needle, for example) for I-RSBT can be fitted with a docking device that can be inserted into a grasping mechanism, or control unit, that can rotate the shaft (e.g., a needle or a catheter). Examples of catheter docking and grasping devices are shown in FIGS. 37A-B.

FIGS. 39A-39C illustrate example embodiments of docking devices in accordance with one or more aspects of the disclosure. As shown in FIGS. 39A-C, the docking device can include a template 200 (FIGS. 39B-C) that contains a clamp 220 (FIGS. 39A & B) around the openings 210 of the template 200. The clamp 220 (FIG. 39A) includes a first series 230 of individual tubes 232. The individual tubes 232 of the first series 230 are positioned around the opening 210 of the template 200 that is configured to receive an I-RSBT needle. The first series 230 of tubes 232 are arranged in parallel with the opening 210. In some instances, the tubes 232 of the first series 230 can form the boundaries of the openings 210. The clamp 220 includes a second set 240 of tubes 242. The second set 240 of tubes 242 can run perpendicularly to the first set 230 of tubes 232. In an aspect, the second set 240 of tubes 242 includes two tubes 242, with one tube 242 abutting a tube 232 of the first series 230 that is positioned on one side of the opening 210, and the other tube 242 abutting a different tube 232 of the first series 230 found on the opposite side of the opening 210. In an aspect, the tubes 242 of the second series 240 can be positioned at the top and the bottom of the opening 210. The tubes 242 of the second series 240 can include an opening 244 that runs the length of the tube 242. The openings 244 can be configured to receive an engaging pin 250.

When an I-RSBT needle is received within an opening 210 of the template 200, an engaging pin 250 can be inserted into the openings 244 of the tubes 242 of the second series 240. The engaging pin 250 applies pressure to the adjacent tube 232 of the first series 230, which prevents the needle from moving inwardly or outwardly of the opening 210 surrounded by the clamp 220. The parallel arrangement of the tubes 232 of the first series 230 allows for the rotational movement of the needle within the opening 210. As shown, the openings 210 of the template 200 can be placed in parallel lines, allowing the tubes 242 of the second series 240 to run the length of the template 200. However, in other aspects, the arrangement of the openings 210 and clamps 220, and the components of the clamps 220, can vary.

In an aspect, the I-RSBT system is not necessarily limited to ⁵⁷Co and ¹⁵³Gd for radiation delivery, although those isotopes are considered to be the most optimal. Other radioisotopes could be used for I-RSBT delivery, including ¹⁹²Ir, ¹³¹Cs, ¹²⁵I, ¹⁰³Pd, ¹⁹⁸Au, ¹⁸⁷W, ¹⁶⁹Yb, ¹⁴⁵Sm, ¹³⁷Cs, ¹⁰⁹Cd, ⁶⁵Zn, ⁵⁶Co, and ⁵⁸Co.

It should be appreciated that the presence of the shielded needles or catheters in the patient's tumor may make imaging of the tumor difficult, with possible introduction of artifacts. In one aspect, a technique (e.g., system, method, etc.) for mitigating or avoiding such difficulty can comprise an I-RSBT system with plastic needles that can be placed in the tumor first for initial positioning and shielded catheters that can then be placed inside the plastic needles after a final, artifact-free, image is acquired to confirm the locations of the catheters. In an aspect, the plastic needles can be placed in the targeted area using known imaging navigation techniques. Such a system is discussed in more detail below.

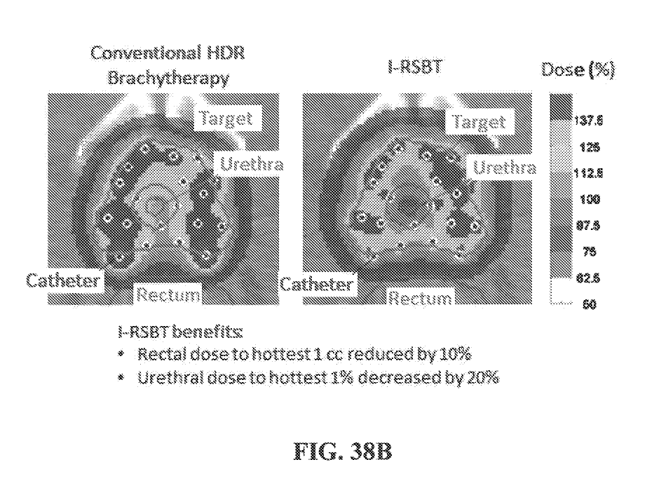

In addition to the prototypes shown in FIGS. 37A-37B and 39A-C, preliminary simulated prostate results shown in FIG. 38A-B demonstrate that I-RSBT is effective in reducing the doses to untargeted areas when compared to conventional HDR-BT. For example, FIG. 38A shows that a shield transmission for the I-RSBT plans was assumed to be 10%, which can be achieved for ¹⁵³Gd and ⁵⁷Co sources using platinum shields with 0.37 mm and 0.71 mm thicknesses, respectively. For the goal of urethral sparing with a prostate D₉₀ (minimum dose to the hottest 90% of the prostate) of 100%, then the maximum urethral dose is reduced from 84% for unshielded BT to 60% for I-RSBT, a 38.5% drop. If the goal is prostate dose escalation while holding maximum urethral dose constant at 100%, then the prostate D₉₀ is increased from 119% for unshielded BT to 167% for I-RSBT, a 40% increase.

FIG. 38B shows that I-RSBT can reduce the rectal dose to the hottest 1 cc by 10% and the urethal dose to the hottest 1% by 20% when compared to conventional HDR-BT. In another aspect, utilizing I-RSBT for treating a prostate tumor can allow for prostate tumor dose escalation, in which the dose to the prostate tumor is increased as much as possible until the urethral tolerance dose is reached. For dose escalation, tumor D₉₀ can be increased by 40% above the previous limits without increasing urethral toxicity above standard HDR-BT levels. Dose escalation can enable a reduction in the number of treatment fractions needed for I-RSBT relative to HDR-BT (currently 2-4), as well as enable a more aggressive treatment regimens for advanced prostate cancer, which has been demonstrated to respond favorably to dose escalation.

In another aspect, various embodiments of RSBT can be applied to in the field of radiation oncology, specifically for the treatment of tumors that are not radially symmetric about some axis. In particular, embodiments related to M-RSBT can be suitable for such application. Several aspects utilizing various embodiments of RSBT are disclosed in Appendices G-H.

FIG. 40a shows MRI-generated 3-D renderings of the anatomy of a patient being treated for cervical cancer, including the tumor and nearby critical structures: bladder, rectum, and sigmoid colon. Typical conventional brachytherapy delivers radiation with an x- or gamma-ray emitting source that travels through a set of rigid tandem-and-ovoid (T&O) applicators inserted into the anesthetized patient. The radially symmetric dose distribution emitted by conventional brachytherapy (BT) sources, however, results in the poor tumor coverage, as shown in FIG. 40b. The desired radiation dose to the tumor, shown as the red outline, is 100% of the prescribed radiation dose, which is clearly not being achieved in a large fraction of the tumor. Improved tumor coverage can be achieved with rotating shield intensity modulated brachytherapy (S-RSBT), which uses shielding of the radiation source to achieve the dose distribution shown in FIG. 40c. It is expected that the improved tumor coverage obtained with S-RSBT to increase local tumor control probability in any applicable tumor, improving patient outcomes.

S-RSBT can be delivered using radioisotopes and the Xoft Axxent electronic brachytherapy source, respectively, by collimating the source with high-density shields that create fan beams. The fan beam source is rotated inside the patient in a manner such that the amount of time the source spends irradiating in a given direction is optimized to ensure better tumor coverage and better critical structure avoidance than conventional brachytherapy (FIG. 40c). Although both works demonstrate the potential benefits of S-RSBT, there is a crippling challenge associated with the single rotating shield approach: the delivery times associated with IMBT are increased relative to conventional BT. This is due to the loss of emitted radiation in the rotating shield, which must remove a large fraction, possibly around 90%, of the radiation in order to achieve an advantage over conventional BT. If the rotating fan beam accounts for only 10% of the radiation emitted by the BT source, with the rest is lost in the shield, then delivering the same dose distribution as conventional BT will require at least ten times as long with rotating-shield IMBT. This is because the fan will have to be pointed in 10 directions and stay pointed in each direction for the same amount of time necessary to deliver an entire conventional BT plan, which loses 0% of the radiation due to shielding.

In one aspect, the disclosed apparatus for Multiple Rotating-Shield IMBT (M-RSBT) can permit the delivery of radiation does distributions with the advantages of S-RSBT, but with substantially lower treatment times. With one or more embodiments of the disclosure, a patient-specific combination of shield emission angles is chosen intelligently to reduce the treatment times while exactly duplicating the dose distribution of S-RSBT. In one aspect, the combination favors large emission angles, so that as little of the emitted radiation is lost as possible, and is determined by computer-based optimization following determination of the tumor shape and applicator by imaging, an example of which is shown in FIG. 40a.

### Delivery Apparatus Description

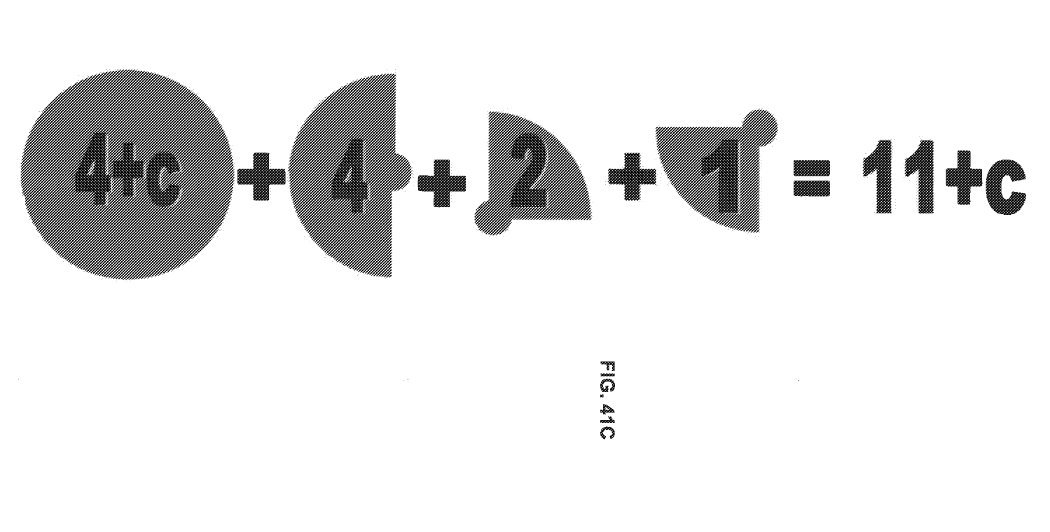

In an aspect, the principle of M-RSBT can be described with reference to FIGS. 41A-41C, which illustrate how the intelligent use of multiple shields can dramatically reduce the treatment time of S-RSBT, while exhibiting the exact same dose distribution. In general, a brachytherapy source is inserted into a source catheter and allowed to dwell at a number of positions within a tumor to apply the treatment. Radiation is emitted by the source in all directions from each dwell positions (i.e., a position in which the BT source stays to apply BT). Using S-RSBT, each direction of the emission angle is required to have a specific dwell time (e.g., FIG. 41B), increasing the specificity of the tumor but drastically increasing treatment time. However, FIGS. 41B-C shows that M-RSBT can achieve tumor specificity with a lower treatment time than S-RSBT.

FIG. 41A-B show a schematic representation of the dwell times according to an example of an application of S-RSBT to a tumor. As shown in FIG. 41A, the S-RSBT utilizes a 90° emission angle in four discrete locations. The four locations are exposed to the S-RSBT (i.e., dwell time), starting at the bottom left and traveling clockwise, for: 9 units, 8 units, 6 units, and 4 units. FIG. 41B shows the total amount of the treatment time using the S-RBST application shown in FIG. 41A: the treatment time equals of 27 units. FIG. 41C illustrates the total time units when M-RSBT is used to treat the same dose distribution. The total treatment time is 11 units plus an extremely small factor “c”, which is less than half the time it takes to treat the same distribution with S-RSBT. “c” is a correction term for the transmission differences, and is described herein. In an aspect, c is equal to 4 T_{shield}, where T_{shield} is the radiation transmission through the shield.

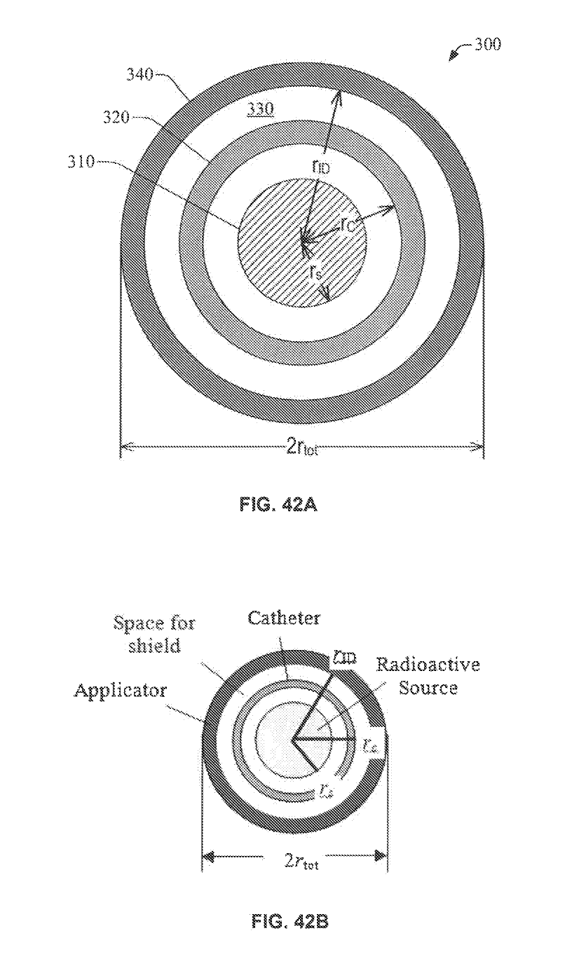

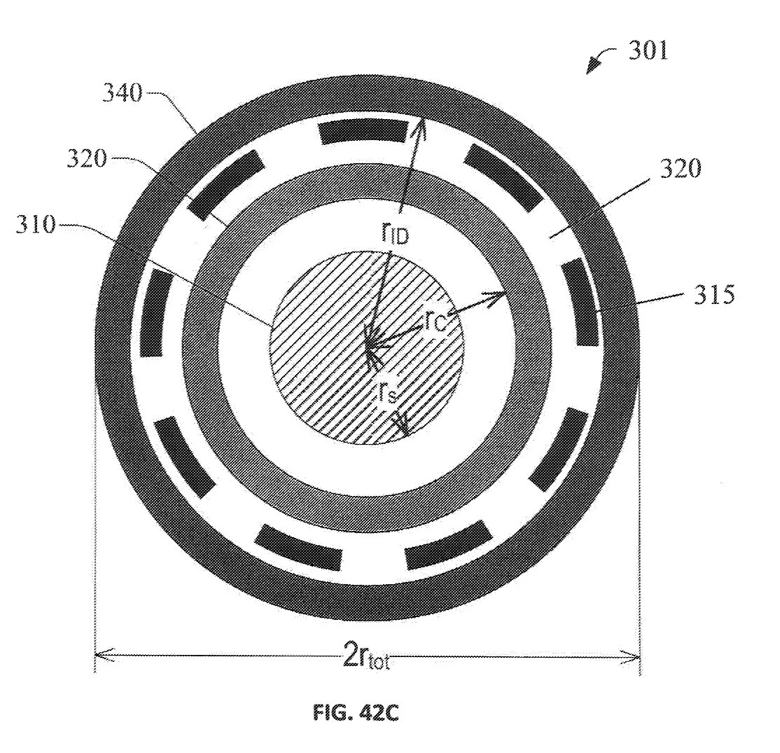

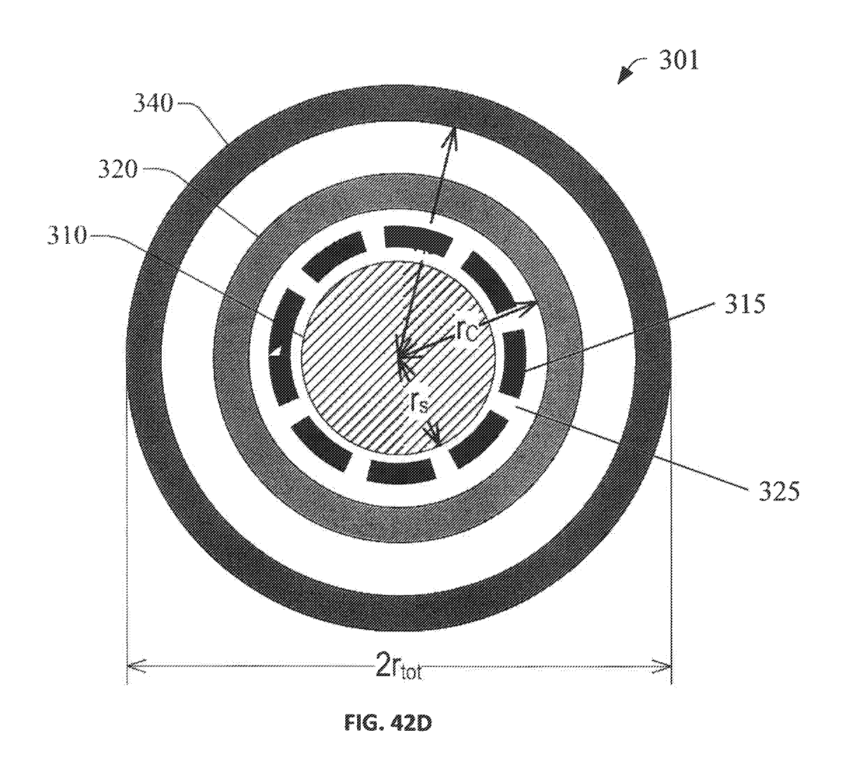

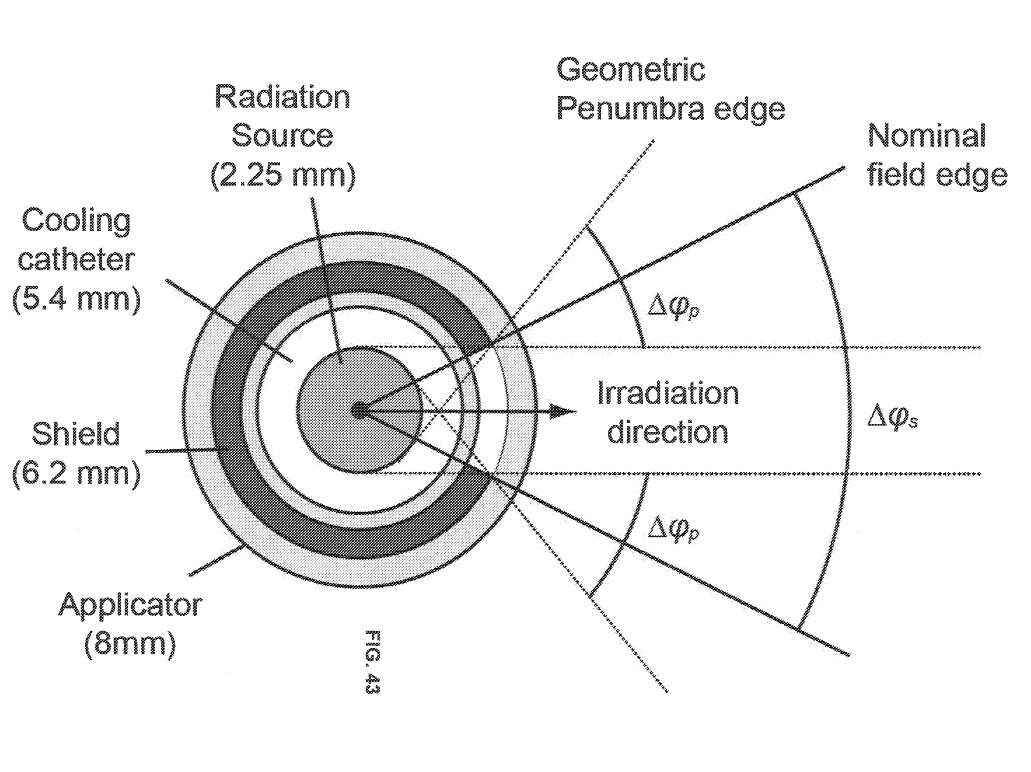

A cross sectional view of an intensity modulated brachytherapy (IMBT) insertion device 301 is shown in FIGS. 42A-42D and FIG. 43, which more comprehensively illustrates the relative locations of a BT source 310, a BT source catheter 320, a shield 315 (or space 330 for one), and applicator 340. In an aspect, the BT source catheter 320 is configured to contain the BT source 310. In an aspect, the shield 315 can include a shield with an emission angle filed with a window, or just the shield with an emission angle. In another aspect, the applicator 340 can be an insertion device that encloses the BT source 310, the BT source catheter 320, and a shield 315. In an aspect, the applicator 340 can already be positioned within a body of a subject. In general, the BT source 310 is inserted in the catheter 320 and allowed to dwell at a number of positions along the axis of the applicator 340. The exemplary aspect of the IMBT insertion device 301 of FIGS. 42A-42D illustrate an intracavitary applicator 340 of inner radius r_{ID} and outer radius rₜₒₜ. The BT/radiation source 310 has an outer radius of rₛ. A BT source catheter tube 320 of outer radius r_{c} may be present, through which the radiation from the BT source 310 travels. The shield may fit in the space 330 between the catheter 320 and insertion device, or, if no BT catheter 320 is used, between the source 310 and surface of the inner applicator 340.

A cross sectional view of an IMBT insertion device 301 is illustrated in FIG. 42A, which depicts the relative locations of the BT source 310, a catheter tube 320 (or catheter 320), a space 330 for a shield in accordance with one or more aspects described herein, and applicator 340. The IMBT insertion device 301 may be embodied in a needle or an intracavitary applicator 340 of inner radius r_{ID} and outer radius rₜₒₜ. In one aspect, the radiation source 310 can have an outer radius rₛ. The radiation source 310 can move through the catheter tube 320, which can have an outer radius r_{c}. In one aspect, the catheter tube 320 forms, at least in part, a first enclosure into which the radiation source 310 can be inserted. In embodiments, such as illustrated in FIG. 42C. a shield 315 (indicated with a thick dashed line) fits in the space 330 between the catheter tube 320 and the applicator 340. More generally, other embodiments of IMBTs can be implemented with ample or sufficient space between r_{c} and r_{ID} for insertion of the shield. It is noted that CBT also can be implemented in embodiments in which no catheter tube 320 is used between the BT source 310 and the inner surface of the applicator 340.

In an aspect, the radiation shield 315 can be coupled to the first enclosure 325 formed by the catheter tube 320 around the source 310, as shown in FIG. 42D. In another aspect, the space 330 is bound by the applicator 340 and the catheter tube 320 and forms a second enclosure that encompasses the first enclosure. As described herein, the catheter 320, which can form the first enclosure, can be adapted to move relative to the second enclosure, defined in part by the applicator 340. As described herein, in one embodiment, the applicator 340 and thus the second enclosure 325, can be coupled to alignment means for positioning the first enclosure (e.g., the catheter tube 320) relative to the second enclosure 325 (e.g., the applicator 340).

FIG. 43 illustrates an additional or alternative example cross section of the RS-IMBT delivery system, with diameters of each component listed. The geometric penumbra edges are shown as dotted lines. The azimuthal shield emission angle is Δφₛ=45°; the geometric penumbra angle is Δφₚ.

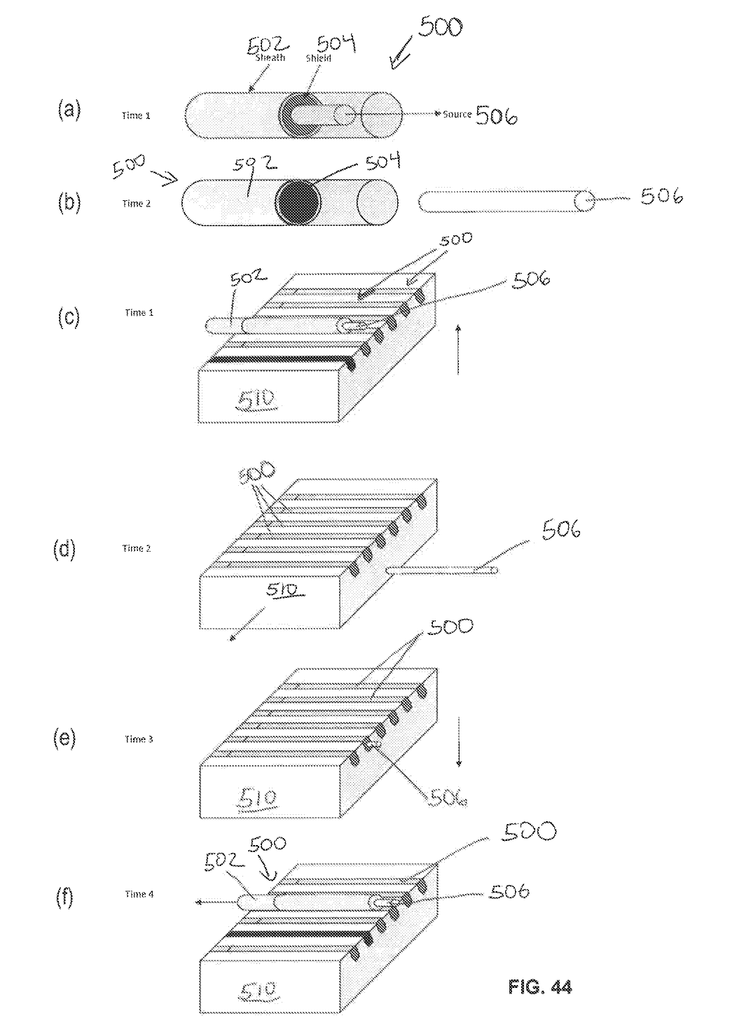

FIG. 44(a-f) illustrate an example apparatus 500 that can be utilized to deliver M-RSBT. The apparatus 500, a combination of a sheath 502 and a shield 504 contained therein, permits changing of sheaths 502 (and therefore shields 504) either automatically (e.g., mechanically, electromechanically, or the like) or manually, especially if electronic radiation sources 506 are being used. A holding block 510 can hold multiple apparatuses 500, with each shield 504 having a different emission angle. The holding block 510, in combination with the apparatuses 500 (already containing different shields 504 within a sheath 502 for easy application of the source 506), allows for the efficient switching between shields 504 and a source 506. In one aspect, the same set of apparatuses 500 can be used for every patient (as shown in FIGS. 44(a-b)), yet the use of the apparatuses 500 would differ allowing each treatment to be highly specific to each patient.

In one aspect, a process by which an individual can change shields 504, by using different apparatuses 500, in order to deliver M-RSBT can comprise the following stages (or actions). As illustrated in FIG. 44(a) during delivery, the source 506 will be inside the sheath 502 and shield 504 of one apparatus 500, and together they will go into the applicator (not shown). As illustrated in FIG. 44(b), during the switching of the source 506, the first apparatus 500 will have the source 506 removed from the sheath 502 and the shield 504. The apparatus 500 with the next appropriate shield 504 will replace it. As illustrated in FIG. 44(c), when the shield 504 needs to be switched, the apparatus 500 will be placed into the holding block 510. In an aspect, the holding block 510 can hold a plurality of apparatuses 500 waiting for use. As shown in FIG. 44(d), the block 510 is then shifted horizontally (shown in the direction of the arrow) so that the next appropriate apparatus 500 aligns with the source 506. As illustration in FIG. 44(e), the source 506 is then inserted into the shield 504 and sheath 502 of the new apparatus 500. As shown in FIG. 44(f), the holding block 510 can be lowered, allowing the new apparatus 500, with the source 506 within it, to be inserted back into the applicator.

### RSBT Treatment Planning

In an aspect, a RSBT treatment plan can be determined. Define a beamlet, \dot{D}_{i,j,k}(Δφ,Δθ), as the dose rate, in Gy/min, at point at \vec{r}ᵢ due to a shielded radiation source at dwell position \vec{s}ⱼ. The shield has an azimuthal emission angle of A, a zenith emission angle of Δθ. The beamlet direction, φₖ, is the azimuthal direction of the center of the emission aperture. Beamlets are patient-dependent in general and can be calculated using techniques such as analytical methods, Monte Carlo methods, solving the radiation transport equation, and interpolation based on pre-calculated or measured dose rate distributions. The beamlet direction is defined as:

φₖ=[mod(k,K)+1/2]δφ,kεZ, (16)

where δφ=360°/K is the azimuthal spacing between beamlet emission angles, K is the total number of azimuthal emission angles per shield rotation considered in the treatment planning problem, and Z is the set of all integers. The modular arithmetic (mod) operator in Equation (16) enables the use of negative k-indices.

For S-RSBT, the total dose delivered to point i is calculated as a time-weighted sum of the beamlets over all dwell positions, indexed by j=0, . . . , J−1 and emission angles:

$${d}_{i} = {\sum }_{j = 0}^{J - 1} {\sum }_{k = 0}^{K - 1} {t}_{j , k} {\dot{D}}_{i , j , k} ( Δϕ , Δθ ) , \qquad ( 17 )$$

where t_{j,k} is the time the source is pointed in direction φₖ while it is located at dwell position \vec{s}ⱼ. The t_{j,k} values are determined using a treatment planning system that optimizes the radiation dose distribution to meet the clinical goal as closely as possible. An example clinical goal is to maximize the minimum dose received by the 90% of the tumor volume receiving the highest dose under the constraint that none of the tolerance doses for any of the radiation-sensitive normal tissues are exceeded.

In certain implementations, it is straightforward to use deterministic (gradient-based, for example) or stochastic (simulated annealing, for example) optimization algorithms to determine t_{j,k} values that produce to a superior dose distribution to that of the unshielded BT case, as long as effective Δθ and Δφ parameters are selected. The total delivery time for the S-RSBT case, tₜₒₜ^{RSBT}, will be strongly influenced by the shield angles selected, and is approximately related to the total delivery time for an unshielded BT source, tₜₒₜ^{BT}, as:

$${t}_{tot}^{RSBT} = {\sum }_{j} {\sum }_{k} {t}_{j , k} ∝ {t}_{tot}^{BT} \frac{180 °}{Δθ} \frac{360 °}{Δϕ} . \qquad ( 18 )$$

Thus an azimuthal shielding angle of Δφ=45° would be expected to result in an S-RSBT delivery time of at least eight-fold that of the unshielded case, even if the zenith emission angle is 180°. For non-radially-symmetric targets, S-RSBT dose distributions will be superior to those of conventional BT, and may be worth some increased cost in delivery time. Multiple rotating shield brachytherapy (M-RSBT), which uses a combination of multiple shields rather than just a single shield, can significantly reduce treatment times below those of S-RSBT. Such a reduction in treatment time can reduce clinical resources by reducing the staffing requirements per patient. For brachytherapy sources such as the Xoft Axxent, which have a finite lifetimes, maximizing delivery efficiency can significantly reduce equipment costs as well.

### Method for M-RSBT Shield Angle and Dwell Time Combinations

With M-RSBT, the radiation dose is delivered with a combination of M different shield emission angles in series. Each shield used in the delivery, indexed by m(m=1, . . . , M), has azimuthal and zenith emission angles of Δφₘ and Δθₘ, respectively, and the total dose distribution is the following:

$${d}_{i} = {\sum }_{m = 1}^{M} {\sum }_{j = 0}^{J - 1} {\sum }_{k = 0}^{K - 1} {\dot{D}}_{i , j , k} ( {Δϕ}_{m} , {Δθ}_{m} ) {t}_{j , k , m} , \qquad ( 19 )$$

which is a generalized version of Equation (17) that includes a sum over all shield angles considered. An m-index is present on the M-RSBT dwell times, t_{j,k,m}, since each shield used in the delivery will have its own set of dwell times for all dwell positions and emission directions. By convention, shield angle increases with shield index m=1, and m=M corresponds to the unshielded case, thus Δφ_{M}=360°. The beamlets corresponding to the shielding hardware shown in FIG. 44 can be used in Equation (19) for M-RSBT treatment planning with standard deterministic or stochastic optimization methods that minimize delivery time without reducing plan quality significantly below that of S-RSBT. The resulting M-RSBT treatment plans will have lower total delivery times, tₜₒₜ^{MRSBT}, than S-RSBT, and comparable dose distributions to S-RSBT.

### Rapid M-RSBT Planning by Combining Neighboring Baseline Beamlets

Suppose a set of baseline beamlets is available for a baseline emission angle, Δφ, and Δφ=δφ. The M-RSBT treatment plan optimization process can be accomplished approximately M-times faster than optimizing Equation (19) directly, based on the recognition that beamlets with emission angles that are integer multiples of Δφ can be constructed by superposing neighboring baseline beamlets. In this section we describe a rapid M-RSBT technique that is based on combining neighboring baseline beamlets into larger beamlets by superposition.

Summing m neighboring baseline beamlets results in a new beamlet with an azimuthal emission angle of m-times Δφ, and an emission direction that is the average of the emission directions of the m combined beamlets. Superposing an odd and even number of neighboring baseline beamlets produces a larger beamlet with a direction that is and is not shared with one of the baseline beamlets, respectively. In order to differentiate between beamlet angles different m-values, we define φₖ^{m} as the emission angle for beamlet k. φₖ^{m} has an azimuthal emission angle of Δφₘ=mΔφ, which can be calculated in general as:

$${ϕ}_{k}^{m} = [ mod ( k , N ) + \frac{1}{2} mod ( m , 2 ) ] δϕ , \qquad ( 20 )$$

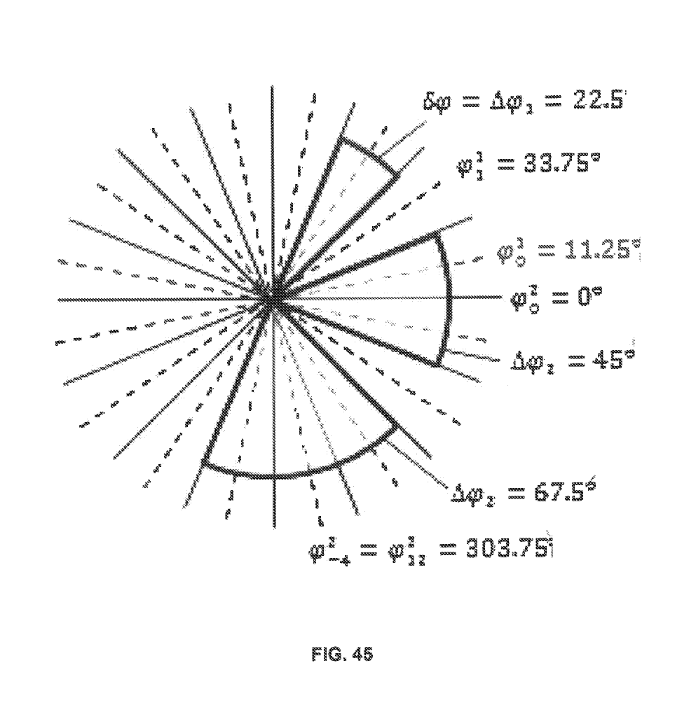

FIG. 45 illustrates examples for the case in which the baseline azimuthal emission angle, Δφ, is equal to the azimuthal emission direction separation, δφ. The figure shows neighboring beamlet superpositions for the cases in which m is 1 and 2, which correspond to superposing 2 and 3 neighboring beamlets, respectively.

In the disclosed approach, the key relationship enabling the construction of beamlets with emission angles larger than the finest angle is the following:

$${\sum }_{{k}^{′} = - ⌊ m / 2 ⌋}^{⌈ m / 2 ⌉ - 1} {\dot{D}}_{i , j , k + {k}^{′}} ( Δϕ , Δθ ) = {\dot{D}}_{i , j , k} ( m Δϕ , Δθ ) + ( m - 1 ) {\dot{D}}_{i , j} ( 0 ° , Δθ ) , \qquad ( 21 )$$

where \dot{D}_{i,j}(0°, Δθ) is the dose rate at point i when the radiation source is located at dwell position j and completely surrounded by the shield. The ┌ ┐ and └ ┘ operators denote ceiling (round-up) and floor (round-down) operations, respectively. Equation (21) holds regardless of the methodology used for calculating \dot{D}_{i,j,k}(Δφ,Δθ), which could vary widely depending on the application. The practical implication of Equation (21) is that m neighboring beamlets with emission angles of Δφ can be superposed and replaced with a single beamlet with an emission angle of φₘ=mΔφ, plus a transmission term. The transmission term is equal to (m−1) times the dose rate at all voxels due to transmission through a completely-shielded source, since adding kernel beamlets (emission angles of Δφ) also adds the radiation transmission values of the kernel beamlets. Shield transmission cannot be neglected in general, but, if the shield is thick enough for transmission to be negligible, then the transmission term vanishes.

The goal of the M-RSBT method is to enable the user to conduct a single optimization with the kernel beamlets, then determine a new set of delivery times using a combination of shielded sources. Producing a set of delivery times that applies to all desired shield emission angles without the need to re-calculate the beamlets or dose distributions for all of the different shield angles is desirable in order to ensure that the algorithm is efficient. The following approximation enables the problem to be solved completely in the space of delivery times:

\dot{D}_{i,j}(0°,Δθ)≅T_{shield}\dot{D}_{i,j}(360°,Δθ), (22)

which enables us to rewrite Equation ( ).

Assume for a given dwell position j that there are m kernel directions, centered on direction k, that have dwell times of at least τ. Mathematically, this can be written as:

$${min}_{k - ⌊ m / 2 ⌋ \leq {k}^{′} \leq k + ⌈ m / 2 ⌉ - 1} {t}_{j , {k}^{′}} = τ , \qquad ( 23 )$$

The m neighboring kernel beamlets can then be superposed into a single beamlet centered on emission direction φₖ and with an emission angle of Δφₘ=mΔφ, plus a transmission term, as follows:

$$τ {\sum }_{{k}^{′} = - ⌊ m / 2 ⌋}^{⌈ m / 2 ⌉ - 1} {\dot{D}}_{i , j , k + {k}^{′}} ( Δϕ , Δθ ) = τ [ {\dot{D}}_{i , j , k} ( m Δϕ , Δθ ) + ( m - 1 ) {T}_{shield} {\dot{D}}_{i , j} ( 0 ° , Δθ ) ] . \qquad ( 24 )$$

Thus the dwell times for M-RSBT can be determined as:

t_{j,k,m}=t_{j,k,m}+τ and

tⱼ^{360°}=tⱼ^{360°}+τ(m−1)T_{shield}, (25)

where tⱼ^{360°} is the dwell time for the unshielded beamlet at dwell position j. The original times t_{j,k} are then decremented as:

t_{j,k′}=t_{j,k′}−τ for k−└m/2┘≦k′≦k−┌m/2┐−1, (26)

and the process repeats for any m-values corresponding to shield emission angles the user has access to. At the end of the dwell time reassignment process for emission angles larger than Δφ, the remaining t_{j,k} values are added to t_{j,k,l}, ensuring all t_{j,k} values are reassigned to t_{j,k,m}.

Circumstances exist for which it is preferable to redistribute the dwell times due to transmission terms amongst the baseline beamlet times rather than directly into the unshielded beamlet. An approximate \dot{D}_{i,j}(360°,Δθ) can now be calculated, the dose rate delivered to all voxels from a shield with a zenith emission angle of Δθ and a 360° azimuthal emission angle by combining kernel beamlets as follows:

$$\begin{matrix}{\sum }_{k = 0}^{K - 1} {\dot{D}}_{i , j , k} ( Δϕ , Δθ ) = {\dot{D}}_{i , j} ( 360 ° , Δθ ) + ( K - 1 ) {\dot{D}}_{i , j} ( 0 ° , Δθ ) \\ ≅ {\dot{D}}_{i , j} ( 360 ° , Δθ ) + ( K - 1 ) {T}_{shield} {\dot{D}}_{i , j} ( 360 ° , Δθ ) \\ = [ 1 + ( K - 1 ) {T}_{shield} ] {\dot{D}}_{i , j} ( 360 ° , Δθ ) ,\end{matrix} \qquad ( 27 )$$

therefore:

$${\dot{D}}_{i , j} ( 360 ° , Δθ ) ≅ \frac{1}{1 + ( K - 1 ) {T}_{shield}} {\sum }_{k = 0}^{K - 1} {\dot{D}}_{i , j , k} ( Δϕ , Δθ ) . \qquad ( 28 )$$

It follows from Equation ( ) that the shielded dose can be calculated using the kernel beamlets as:

$${\dot{D}}_{i , j} ( 0 ° , Δθ ) ≅ \frac{{T}_{shield}}{1 + ( K - 1 ) {T}_{shield}} {\sum }_{k = 0}^{K - 1} {\dot{D}}_{i , j , k} ( Δϕ , Δθ ) . \qquad ( 29 )$$

Equation (29) can be substituted into the first line of Equation (24) to obtain:

$$τ {\sum }_{{k}^{′} = - ⌊ m / 2 ⌋}^{⌈ m / 2 ⌉ - 1} {\dot{D}}_{i , j , k + {k}^{′}} ( Δϕ , Δθ ) = τ {\dot{D}}_{i , j , k} ( m Δϕ , Δθ ) + {\sum }_{k = 0}^{K - 1} [ \frac{τ ( m - 1 ) {T}_{shield}}{1 + ( K - 1 ) {T}_{shield}} ] {\dot{D}}_{i , j , k} ( Δϕ , Δθ ) . \qquad ( 30 )$$

Thus the dwell times for M-RSBT can be to account for the reassignment to a larger emission angle beamlet and radiation transmission from the baseline beamlets as follows:
The original times t_{j,k} are then decremented as:

$${t}_{j , k , m} = {t}_{j , k , m} + τ \\ {t}_{j , k , 1} = {t}_{j , k , 1} + τ \frac{( m - 1 ) {T}_{shield}}{1 + ( K - 1 ) {T}_{shield}} for all {k}^{′} \qquad ( 31 )$$

t_{j,k′}=t_{j,k′}−τ for k−└m/2┘≦k′≦k−┌m/2┐−1, (32)

Data and/or simulation demonstrate, in one aspect, that the treatment time using the M-RSBT delivery method will always be as short as or shorter than could be obtained using S-RSBT. In one aspect, the radiation source was modeled as a Xoft Axxent electronic brachytherapy (eBT) source and assumed the system of shields allowed 0% transmission. The RSBT treatment plans were generated using a dose calculator developed at the University of Iowa Hospital & Clinics' Radiation Oncology Department using MATLAB (2009b, The MathWorks, Natick, Mass.). The prescription was set to 100% for all voxels on the tumor surface, and restrictions were set such that the maximum dose for any voxel on the surface of the bladder, sigmoid, and rectum were 90%, 75%, and 75% respectively. Only the surface voxels were considered in the optimization since the source position ensures that the dose inside the tumor will always be greater than the dose delivered at the surface. Similarly, it should be appreciated that the inside of the Organs at Risk (OARs) can always be less than at the surface due to the source position. It also should be appreciated that the therapy advantages (such as reduced treatment time) of M-RSBT with respect to other RSBT techniques can offset the complexity associated with changing the size of the radiation shield during delivery.

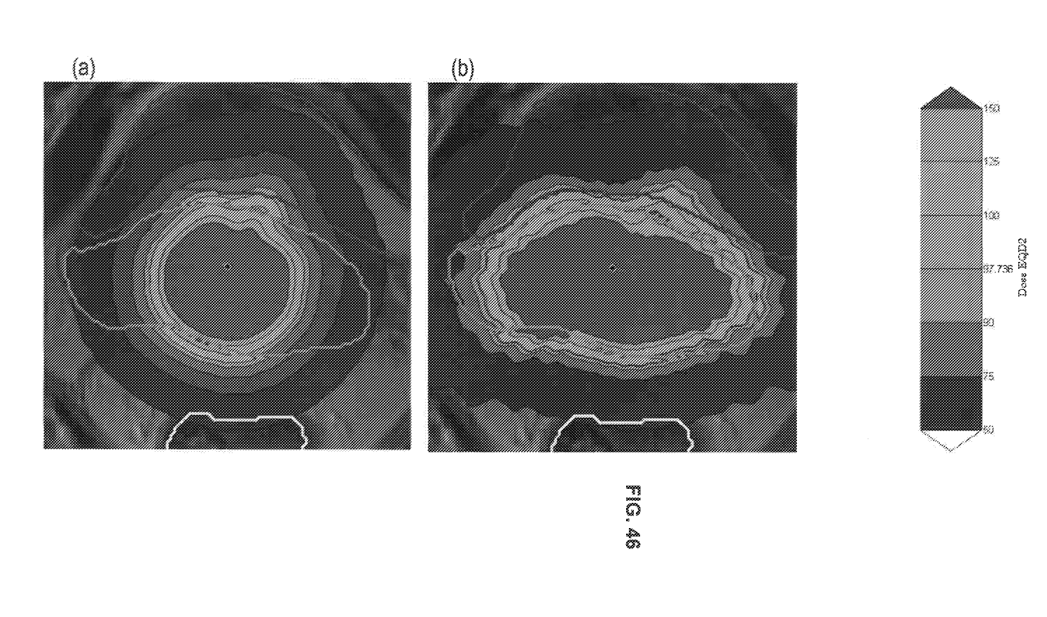

The dose distributions for the patient that would benefit least from M-RSBT out of all the patients tested using a minimum emission angle of 180° and 22.5° are shown in FIG. 46(a) and FIG. 46(b), respectively. Tumor coverage is much better using the smaller emission angle, but but treatment time increased. This patient is not the ideal candidate for M-RSBT because less recombination into the larger emission is possible, yet still consistently yields a shorter treatment time when the M-RSBT method is used.

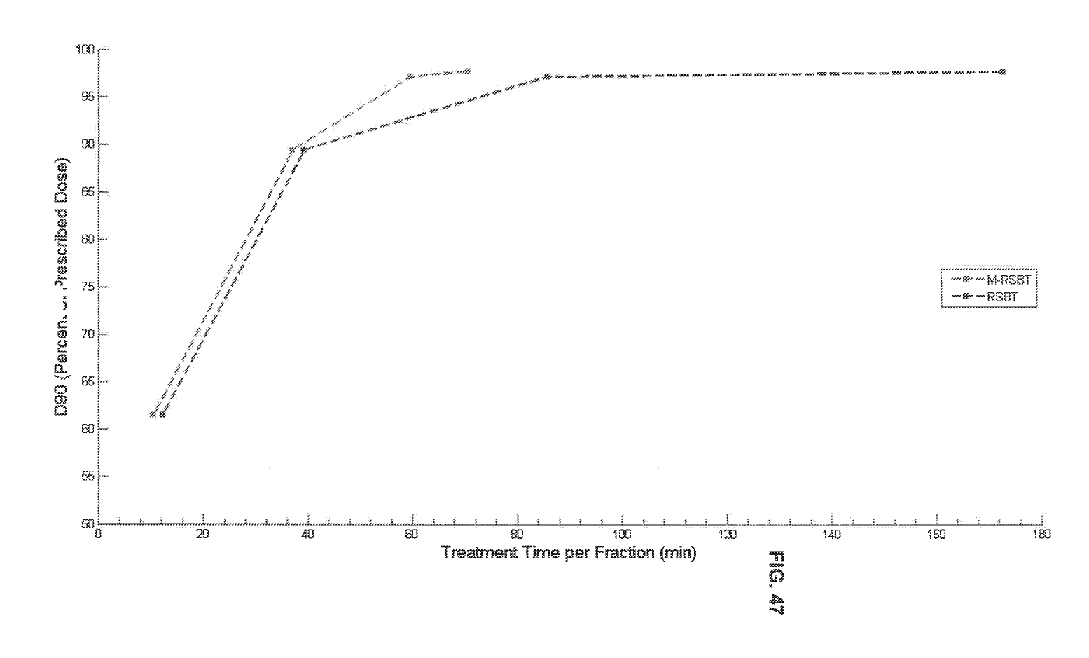

It is observed that the treatment times for this patient, though not the ideal for M-RSBT are always shorter than when using RSBT, as demonstrated in FIG. 47. In addition, as the minimum emission angle gets smaller, M-RSBT increasingly outperforms RSBT. M-RSBT treatment times (green) and RSBT treatment times (blue) plotted against the D₉₀ for the tumor surface. The figure shows that as the D₉₀ gets closer to 100% for the tumor surface (a result of lowering the emission angle) M-RSBT has significantly shorter treatment times than RSBT. However, even at large emission angles M-RSBT outperforms RSBT because some recombination is still possible. The emission angles used for this figure were 180°, 90°, 45°, and 22.5°.

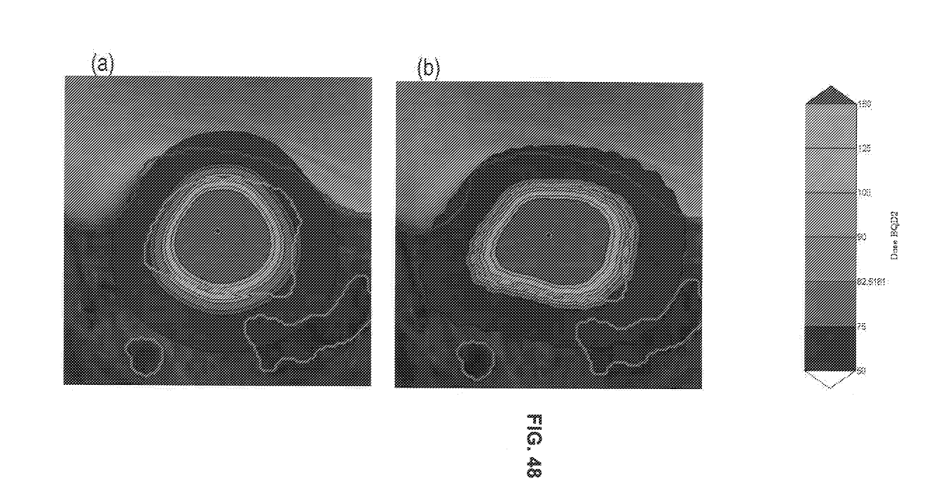

Similar performance is readily available for a patient with a tumor with a high potential for recombination, such as the tumor shown in FIG. 48(a) and FIG. 48(b). FIG. 48(a) shows a dose distribution for a patient that would benefit significantly from M-RSBT using a minimum emission angle of 180°. FIG. 48(b) illustrates the analogous distribution using a minimum emission angle of 22.5°. Tumor coverage is much better using the smaller emission angle.

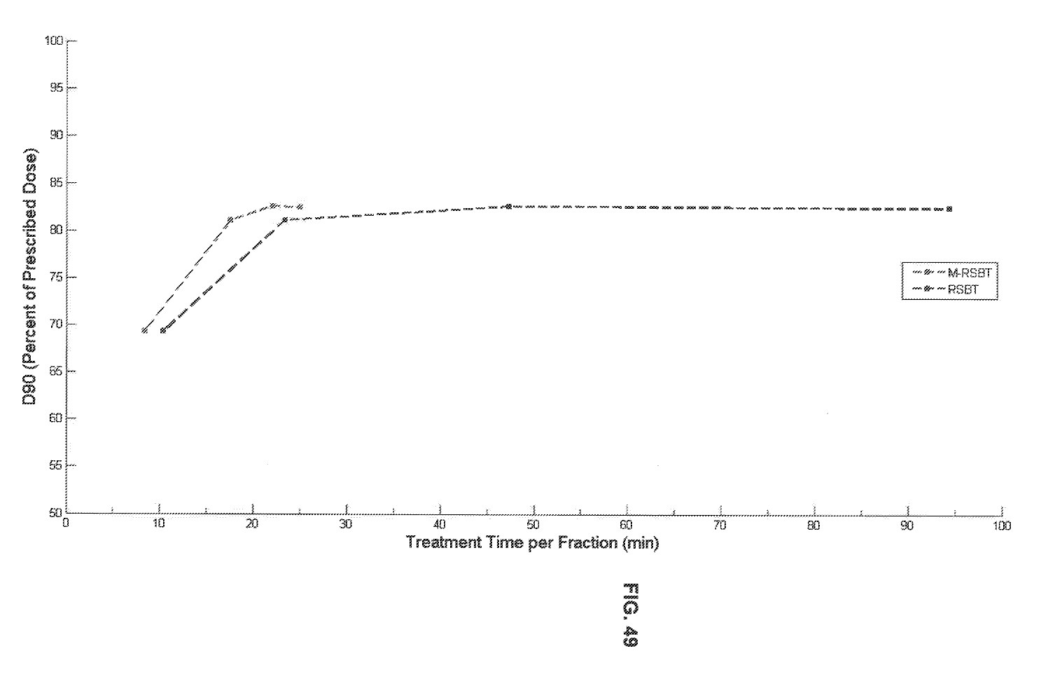

FIG. 49 shows the treatment times for the patient from FIG. 48(a-b), with M-RSBT treatment times (green) and RSBT treatment times (blue) plotted against the D₉₀ for the tumor surface. The figure shows that M-RSBT is substantially more efficient for all emission angles. The emission angles used for this figure were 180°, 90°, 45°, and 22.5°.

As disclosed herein, in one aspect, for every patient and every emission angle, M-RSBT had a shorter treatment time than when using the RSBT method, especially when the emission angle is small enough to provide a satisfactory D₉₀ for the tumor surface.

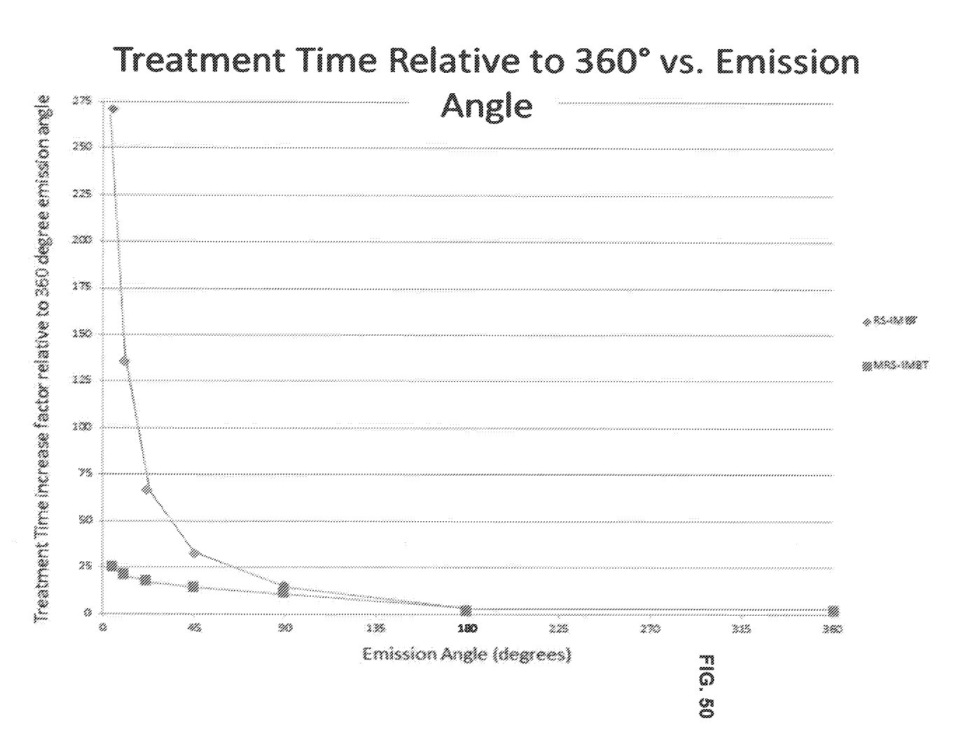

FIG. 50 illustrates an example comparison of treatment times for RSBT and M-RSBT (also referred to as MRS-IMBT) in accordance with one or more aspects of the disclosure.

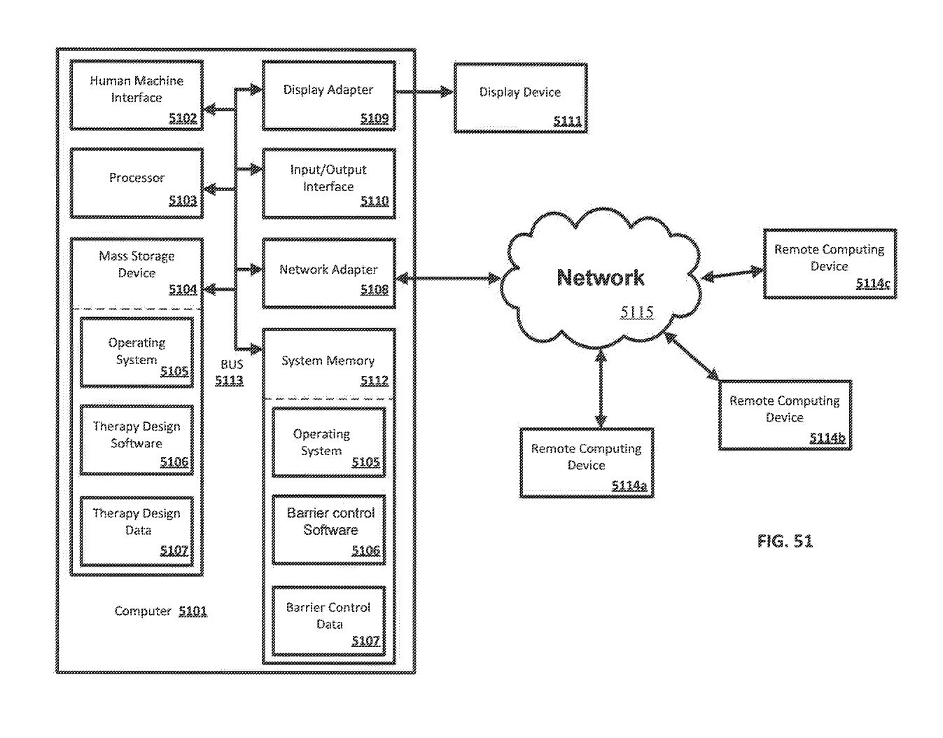

FIG. 51 illustrates a block diagram of an exemplary operating environment 5100 that enables the implementation of therapy design (e.g., treatment plan, beamlet selection construction, radiation shield design, optimization of treatment plan, etc.) and other various features of the subject disclosure and performance of the various methods disclosed herein. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The various embodiments of the subject disclosure can be operational with numerous other general purpose or special purpose computing system environments or configurations. Examples of well-known computing systems, environments, and/or configurations that can be suitable for use with the systems and methods comprise, but are not limited to, personal computers, server computers, laptop devices or handheld devices, and multiprocessor systems. Additional examples comprise wearable devices, mobile devices, set top boxes, programmable consumer electronics, network PCs, minicomputers, mainframe computers, distributed computing environments that comprise any of the above systems or devices, and the like.

The processing effected in the disclosed systems and methods can be performed by software components. The disclosed systems and methods can be described in the general context of computer-executable instructions, such as program modules, being executed by one or more computers or other computing devices. Generally, program modules comprise computer code, routines, programs, objects, components, data structures, etc. that perform particular tasks or implement particular abstract data types. The disclosed methods also can be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 5101. The components of the computer 5101 can comprise, but are not limited to, one or more processors 5103, or processing units 5103, a system memory 5112, and a system bus 5113 that couples various system components including the processor 5103 to the system memory 5112. In the case of multiple processing units 5103, the system can utilize parallel computing.

In general, a processor 5103 or a processing unit 5103 refers to any computing processing unit or processing device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally or alternatively, a processor 5103 or processing unit 5103 can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors or processing units referred to herein can exploit nano-scale architectures such as, molecular and quantum-dot based transistors, switches and gates, in order to optimize space usage or enhance performance of the computing devices that can implement the various aspects of the subject disclosure. Processor 5103 or processing unit 5103 also can be implemented as a combination of computing processing units.

The system bus 5113 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 5113, and all buses specified in this description also can be implemented over a wired or wireless network connection and each of the subsystems, including the processor 5103, a mass storage device 5104, an operating system 5105, therapy design software 5106, therapy design data 5107, a network adapter 5108, system memory 5112, an Input/Output Interface 5110, a display adapter 5109, a display device 5111, and a human machine interface 5102, can be contained within one or more remote computing devices 5114a,b,c at physically separate locations, connected through buses of this form, in effect implementing a fully distributed system.

In one aspect, therapy design software 5106 can comprise computer-executable instructions for implementing the various methods described herein; in particular, yet not exclusively, the various methods described herein. In another aspect, therapy design software 1706 can include software to control various aspects of manufacturing of the shield and, as part of manufacturing, treating a surface in accordance with aspects described herein in order to attain a desired thickness profile for the surface of the shield. In certain embodiments, therapy design software 5106 also can include computer-executable instruction for selecting radio-opaque materials for manufacturing the shield. Therapy design software 5106 and therapy design data 5107 (which can comprise radiation shield data) can configure processor 5103 to perform the one or more steps (or stages or actions) of the methods described herein. In addition or in the alternative, therapy design software 5106 and therapy design data 5107 can configure processor 5103 to operate in accordance with various aspects of the subject disclosure.

The computer 5101 typically comprises a variety of computer readable media. Exemplary readable media can be any available media that is accessible by the computer 5101 and comprises, for example and not meant to be limiting, both volatile and non-volatile media, removable and non-removable media. The system memory 5112 comprises computer readable media in the form of volatile memory, such as random access memory (RAM), and/or non-volatile memory, such as read only memory (ROM). The system memory 5112 typically contains data and/or program modules such as operating system 5105 and therapy design software 5106 that are immediately accessible to and/or are presently operated on by the processing unit 5103. Operating system 5105 can comprise OSs such as Windows operating system, Unix, Linux, Symbian, Android, iOS, Chromium, and substantially any operating system for wireless computing devices or tethered computing devices.

In another aspect, the computer 5101 also can comprise other removable/non-removable, volatile/non-volatile computer storage media. By way of example, FIG. 51 illustrates a mass storage device 5104 which can provide non-volatile storage of computer code, computer readable instructions, data structures, program modules, and other data for the computer 5101. For example and not meant to be limiting, a mass storage device 5104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 5104, including by way of example, an operating system 5105, and therapy design software 5106. Each of the operating system 5105 and therapy design software 5106 (or some combination thereof) can comprise elements of the programming and the therapy design software 5106. Data and code (e.g., computer-executable instruction(s)) can be retained as part of therapy design software 5106 and can be stored on the mass storage device 5104. Therapy design software 5106, and related data and code, can be stored in any of one or more databases known in the art. Examples of such databases comprise, DB2®, Microsoft® Access, Microsoft® SQL Server, Oracle®, mySQL, PostgreSQL, and the like. Further examples include membase databases and flat file databases. The databases can be centralized or distributed across multiple systems.

In another aspect, the user can enter commands and information into the computer 5101 via an input device (not shown). Examples of such input devices comprise, but are not limited to, a camera; a keyboard; a pointing device (e.g., a “mouse”); a microphone; a joystick; a scanner (e.g., barcode scanner); a reader device such as a radiofrequency identification (RFID) readers or magnetic stripe readers; gesture-based input devices such as tactile input devices (e.g., touch screens, gloves and other body coverings or wearable devices), speech recognition devices, or natural interfaces; and the like. These and other input devices can be connected to the processing unit 5103 via a human machine interface 5102 that is coupled to the system bus 5113, but can be connected by other interface and bus structures, such as a parallel port, game port, an IEEE 1394 Port (also known as a Firewire port), a serial port, or a universal serial bus (USB).

In yet another aspect, a display device 5111 also can be connected to the system bus 5113 via an interface, such as a display adapter 5109. It is contemplated that the computer 5101 can have more than one display adapter 5109 and the computer 5101 can have more than one display device 5111. For example, a display device can be a monitor, an LCD (Liquid Crystal Display), or a projector. In addition to the display device 5111, other output peripheral devices can comprise components such as speakers (not shown) and a printer (not shown) which can be connected to the computer 5101 via Input/Output Interface 5110. Any step and/or result of the methods can be output in any form to an output device. Such output can be any form of visual representation, including, but not limited to, textual, graphical, animation, audio, tactile, and the like.

The computer 5101 can operate in a networked environment using logical connections to one or more remote computing devices 5114a,b,c. By way of example, a remote computing device can be a personal computer, portable computer, a mobile telephone, a server, a router, a network computer, a peer device or other common network node, and so on. Logical connections between the computer 5101 and a remote computing device 5114a,b,c can be made via a local area network (LAN) and a general wide area network (WAN). Such network connections can be through a network adapter 5108. A network adapter 5108 can be implemented in both wired and wireless environments. Such networking environments are conventional and commonplace in offices, enterprise-wide computer networks, intranets, and the Internet. Networking environments are referred to as network(s) 5115 and generally can be embodied in wireline networks or wireless networks (e.g., cellular networks, such as Third Generation (3G) and Fourth Generation (4G) cellular networks, facility-based networks (femtocell, picocell, Wi-Fi networks, etc.).

As an illustration, application programs and other executable program components such as the operating system 5105 are illustrated herein as discrete blocks, although it is recognized that such programs and components reside at various times in different storage components of the computing device 5101, and are executed by the data processor(s) of the computer. An implementation of therapy design software 5106 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer-readable media can comprise “computer storage media,” or “computer-readable storage media,” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In various embodiments, the systems and methods of the subject disclosure for implementation of advanced rotating-shield brachytherapy can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g., genetic algorithms), swarm intelligence (e.g., ant algorithms), and hybrid intelligent systems (e.g., Expert inference rules generated through a neural network or production rules from statistical learning).

In an aspect, exemplary systems and methods can comprise a radiation source wire designed to contain a sufficient quantity of the gadolinium-153 (¹⁵³Gd) radioisotope for high-dose-rate brachytherapy of cancerous tumors. The distal portion of the source wire can comprise a plugged capsule containing the ¹⁵³Gd, which is welded to a wire having a length between 20 cm and 2 m, and which can be controlled with a remote afterloading system. In one example, the wire can be 30 cm and can be administered by manually or robotically inserting it into a shielded or unshielded catheter. As ¹⁵³Gd sources will have a lower dose rate than ¹⁹²Ir high-dose-rate sources, a radiation plan can be administered by inserting a plurality of ¹⁵³Gd source wires into a plurality of shielded catheters, each of which can be individually controlled by a rotating, translating, motor. All of the metals associated with the wire besides the ¹⁵³Gd can comprise NiTi (nitinol), for strength and flexibility, or stainless steel. In order to construct a source wire, the open ¹⁵³Gd capsule can be filled with the radioactive material and then sealed with a NiTi (or stainless steel) plug, for example, by laser-welding to the capsule opening. The invention can be useful in the fields of Radiation Oncology and Urology, and can be described as a new form of Brachytherapy called Intensity Modulated Brachytherapy (IMBT), rotating shield brachytherapy (RSBT), or dynamically modulated brachytherapy (DMBT). Intensity modulated Brachytherapy can significantly improve radiation dose distribution for brachytherapy patients, especially those with cervical cancer, colorectal cancer, liver cancer, lung cancer, and prostate cancer. The systems and methods can be of commercial value because the systems and methods provide a novel radiation source with an ideal gamma ray energy spectrum that enables patient-specific shielding and similar penetration in tissue to the gamma rays of the ¹⁹²Ir isotope, and these properties are ideal for IMBT. The ¹⁵³Gd isotope emits radiation with an optimal energy spectrum, at an acceptable dose rate, and with a half-life of 240 days. Existing Iridium-192 (¹⁹²Ir) source wires have sub-optimal energy spectra for IMBT.

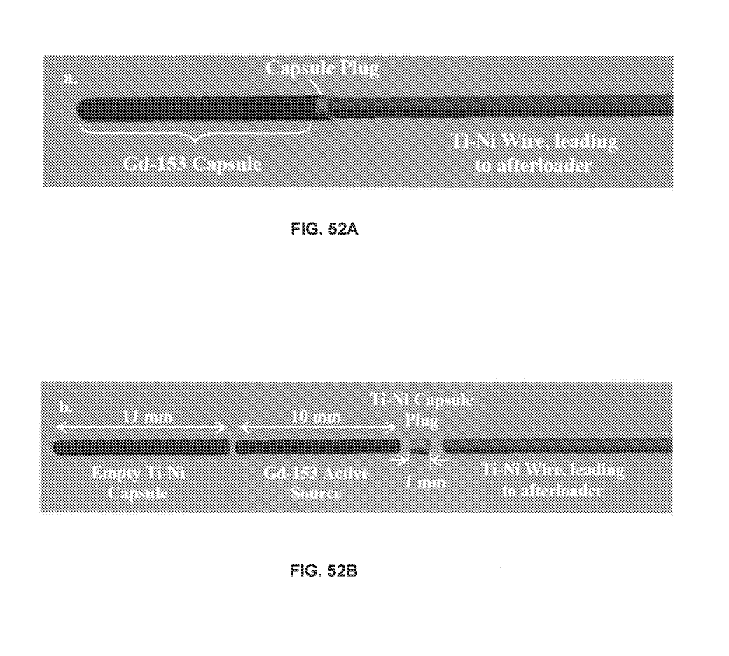

In an aspect, the methods and systems can be applied to Radiation Oncology, and, when applied for prostate cancer treatment, Urology. An exemplary system can comprise a source wire apparatus containing ¹⁵³Gd, shown in FIG. 52. FIG. 52(a) is an exemplary apparatus. The apparatus can comprise an assembled source wire. The assembled source wire can comprise a capsule. In an aspect, the capsule can comprise a radiation source. An example radiation source is ¹⁵³Gd. The assembled source wire can also comprise a plug. The assembled source wire can further comprise a wire attached to the capsule. FIG. 52(b) is an exploded view of source wire. In an aspect, laser welding can be used to connect the Ti—Ni parts together, which encapsulates the ¹⁵³Gd active source. The diameter of the active ¹⁵³Gd source can be 0.3-0.9 mm, and the diameter of the capsule, thick part of the plug, and wire can be 0.5-1.0 mm. The space between the active source and the outer source capsule consists of encapsulation material, which can be NiTi or stainless steel, for example. Encapsulation thicknesses range from 0.05 mm to 0.125 mm. Active source lengths can range from 5 mm to 30 mm. The diameter can be configured for compatibility with an afterloader system such as the GammaMed Plus afterloader system, currently sold by Varian. It should be noted that though FIGS. 52(a)-(b) illustrate particular dimensions and materials, other dimensions and materials can be used.

The wire can be used to deliver high-dose-rate Brachytherapy using a remote afterloading system, which can mechanically control the location of the radiation source wire inside of catheters or applicators that are inserted into a patient. The invention is novel because it is the first such source wire to use the ¹⁵³Gd radioisotope to deliver the radiation dose.

Single or multiple ¹⁵³Gd wires can be used simultaneously with rotating shielded catheters to deliver low-dose-rate (0.4-2 Gy/h), medium-dose-rate (2-12 Gy/h), and high-dose-rate (>12 Gy/h) I-RSBT.

The specific problem the invention can overcome is that of delivering intensity modulated Brachytherapy (I-RSBT) with a radiation source that emits gamma rays in the 100 keV energy range. ¹⁵³Gd emits primarily gamma rays between 40 and 100 keV, which are ideal for enabling the construction of novel shielding systems that can be built into Brachytherapy catheters. The tenth-value-layer, which is the thickness of a shielding material to reduce the radiation dose to 10% of it sun shielded value, is only 0.37 mm of platinum for ¹⁵³Gd, and over 11 mm for ¹⁹²Ir, which is the conventional high-dose-rate brachytherapy isotope. In an aspect, the systems and methods can comprise fitting at least one tenth-value-layer of shielding material inside the catheters through which the source wire will travel in order to deliver I-RSBT effectively. Since ¹⁹²Ir, the conventional high-dose-rate brachytherapy isotope, has such a large tenth-value-layer, it is infeasible to construct shielded catheters that are small enough to treat cancers such as prostate cancer, which involve catheters of 2 mm in diameter or less.

Determining an isotope that is acceptable for I-RSBT has historically been a difficult problem. The concept of single-catheter I-RSBT was proposed in 2002, and multiple catheter I-RSBT was proposed in 2006, yet it was never made clear what isotope could be used to best enable I-RSBT. Low-energy isotopes have been considered for I-RSBT, but the sources have been electronic brachytherapy sources with diameters too large for interstitial applications, which has a relatively short half-life of 59.4 days. Low energy sources also can have the disadvantage of more rapid dose fall-off with distance from the source than higher energy sources, which can increase the magnitude of radiation dose hotspots in the patient. We propose using ¹⁵³Gd for I-RSBT due to its ideal gamma ray energy emission spectrum, long half-life, and recently developed potential for mass production.

Gadolinium-153 (¹⁵³Gd) is not the only isotope that emits gamma rays in the energy range of interest, and it is not obvious that it would be the ideal radioisotope for I-RSBT. According to a previous analysis by Oak Ridge National Laboratory for a very different purpose (atmospheric density measurements), there are several other candidate isotopes, including ⁵⁷Co, ^{91m}Nb, ¹⁰¹Rh, ¹⁵¹Gd, ¹⁶⁸Tm, ¹⁷³Lu, ¹⁷⁴Lu, ¹⁹⁵Au, ^{97m}Tc, ^{99m}Tc, ⁹³Mo, ^{113m}Cd, ¹⁸⁸W, ¹³⁹Ce, ^{123m}Te, ¹²⁵Te, ^{127m}Te, ¹⁷⁰Tm, ¹⁵⁵Eu, and ¹⁰⁹Cd. Through scientific research, ¹⁵³Gd is discovered to be an ideal radiation source for I-RSBT. For example, other candidates can be too costly to produce (^{91m}Nb, ¹⁰¹Rb, ¹⁵¹Gd, ¹⁶⁸Tm, ¹⁷³Lu, ¹⁷⁴Lu, and ¹⁹⁵Au), can have too low specific activities or gamma ray yield (^{97m}Tc, ⁹³Mo, ¹⁰⁹Cd, ^{113m}Cd, and ¹⁷⁰Tm, ¹⁵⁵Eu), can have too short half-lives (^{99m}Tc), can have high-energy gamma ray contamination (¹⁸⁸W) or the presence of other contaminants (¹³⁹Ce), or can provide calibration difficulties (^{123m}Te, ¹²⁵Te, and ^{127m}Te). For example, other similar radiation sources such as ⁵⁷Co can likely be produced in large enough quantities for high-dose-rate brachytherapy, but at an estimated ten times the cost of ¹⁵³Gd.

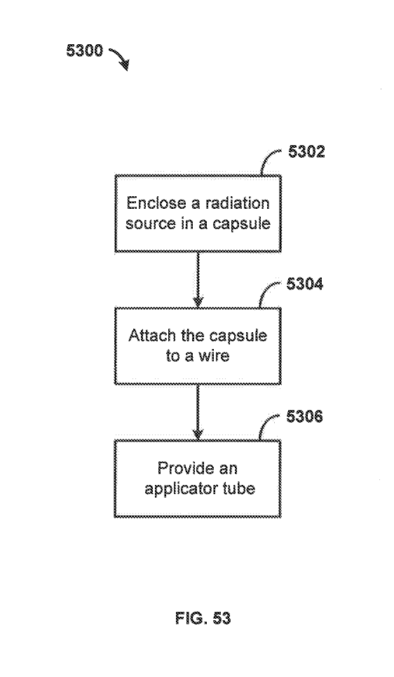

FIG. 53 is a flowchart illustrating an exemplary method 5300 of forming a therapeutic radiation capsule. In step 5302, a radiation source can be enclosed in a capsule. In step 5304, the capsule can be attached to a wire. The wire can be configured to guide the capsule to a target through an applicator tube. In step 5306, an applicator tube configured to guide the capsule to a target can be provided.

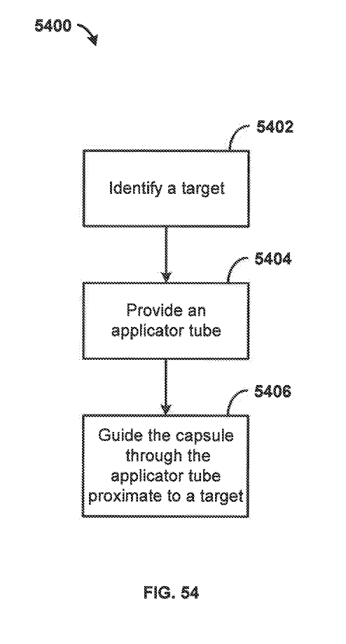

FIG. 54 is a flowchart illustrating an exemplary method 5400 of providing therapeutic radiation. In step 5402, a target can be identified. In step 5404, an applicator tube configured to guide a capsule proximate to the target can be provided. In an aspect, the capsule can enclose the radiation source and have a wire attached to the capsule. In step 5406, the capsule can be guided through the applicator tube proximate to the target by use of the wire.

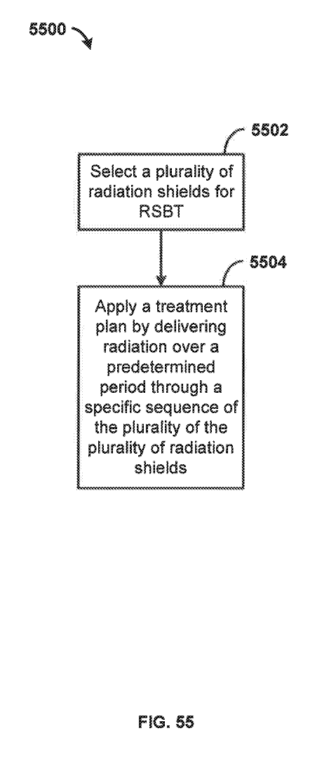

FIG. 55 is a flowchart illustrating an exemplary method 5500 for rotating shield brachytherapy (RSBT). In step 5502, a plurality of radiation shields for RSBT can be selected. In an aspect, each one of the plurality of radiation shields having a specific radiation emission angle. In step 5504, a treatment plan can be applied by delivering radiation over a predetermined period through a specific sequence of the plurality of the plurality of radiation shields.

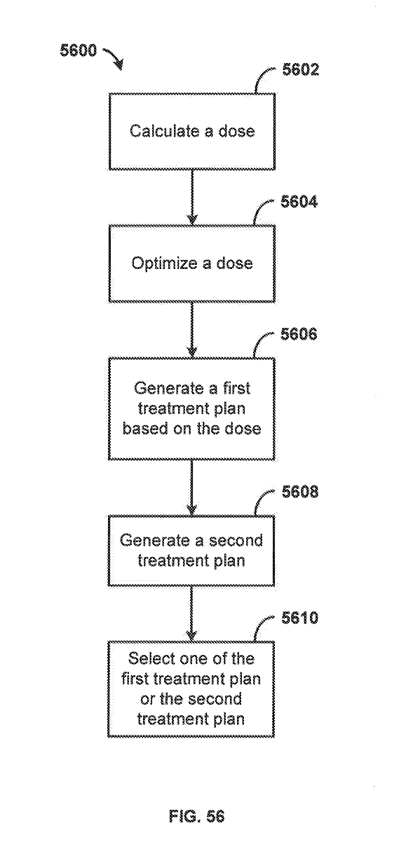

FIG. 56 is a flowchart illustrating an exemplary method 5600 for selecting an emission angle for use in single rotating-shield brachytherapy. In step 5602, a dose can be calculated. In step 5604, the calculated dose can be optimized. In step 5606, a first treatment plan can be generated based on the optimized dose. In step 5608, a second treatment plan can be generated. In step 5610, one of the first treatment plan or the second treatment plan can be selected.

FIG. 57 is a flowchart illustrating an exemplary method 5700 for sequencing rotating shields. In step 5702, a dose is calculated. In step 5704, the dose can be optimized. In step 5706, a treatment plan is generated based on an optimal sequence of the dose.

### I-RSBT Catheter Delivery System

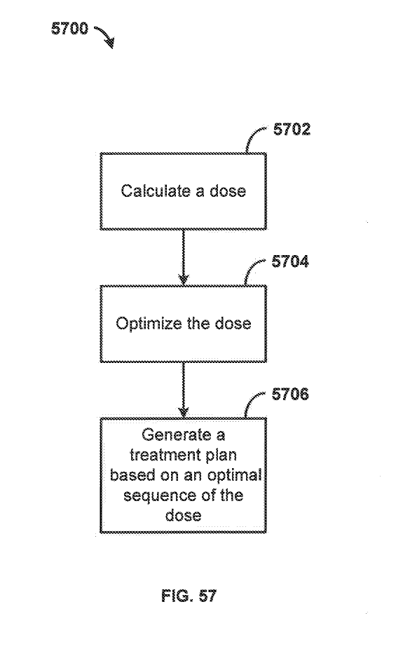

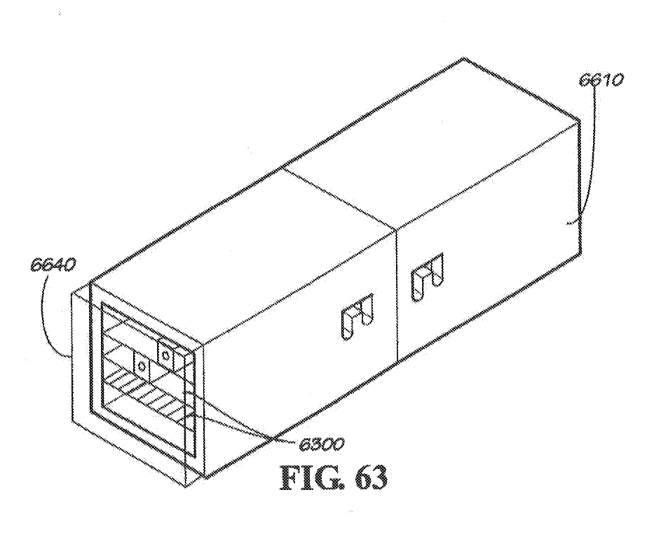

In an aspect, an I-RSBT catheter delivery system 6000 is shown in FIGS. 58-71, which illustrates how the intelligent use of multiple I-RSBT catheters can dramatically reduce the treatment time of S-RSBT. In general, the brachytherapy sources are inserted into multiple catheters which are then positioned at multiple positions within a targeted location within a human subject efficiently. Radiation is emitted by the sources in all directions from each dwell positions. In another aspect, the I-RSBT catheter delivery system 6000 can emit the radiation in a helical pattern, increasing the efficiency of the emission, as well as limiting the exposure for healthy tissues.

In an aspect, as shown in FIGS. 58-64 and 67-68, the I-RSBT catheter delivery system 6000 can include catheter control cartridges 6100 arranged within a cartridge magazine holder 6600 and controlled by a system controller 6700. The catheter control cartridges 6100, controlled by the system controller 6700, engage needles 6500 within a subject to deliver the I-RSBT. Given the nature of I-RSBT, the I-RSBT catheter delivery system 6000 must be able to deliver I-RSBT in numerous precise locations over a very small volume, generally only an area that is 30-100 cm³. Therefore, the catheter control cartridges 6100, which deliver the I-RSBT, can be small in size as well. While the cross sectional size of the catheter control cartridges 6100 can vary from aspect to aspect, in a preferred aspect, the catheter control cartridges 6100 can have a cross section of approximately 9.5 mm×9.5 mm.

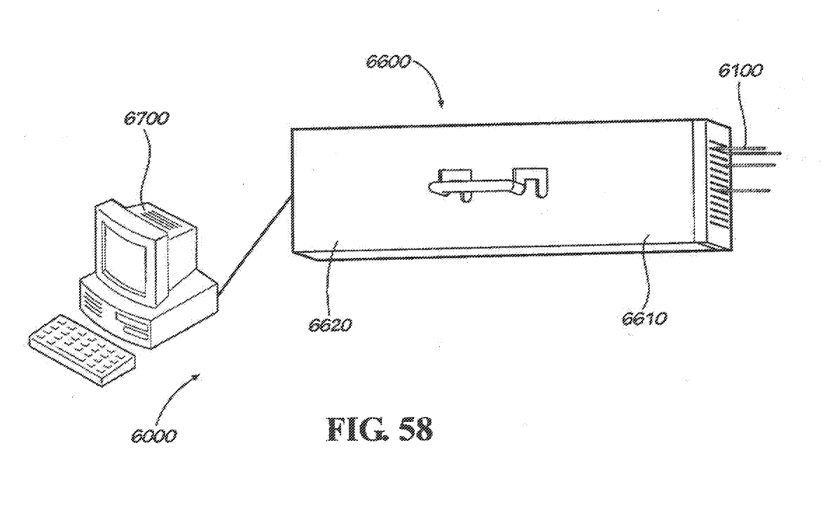

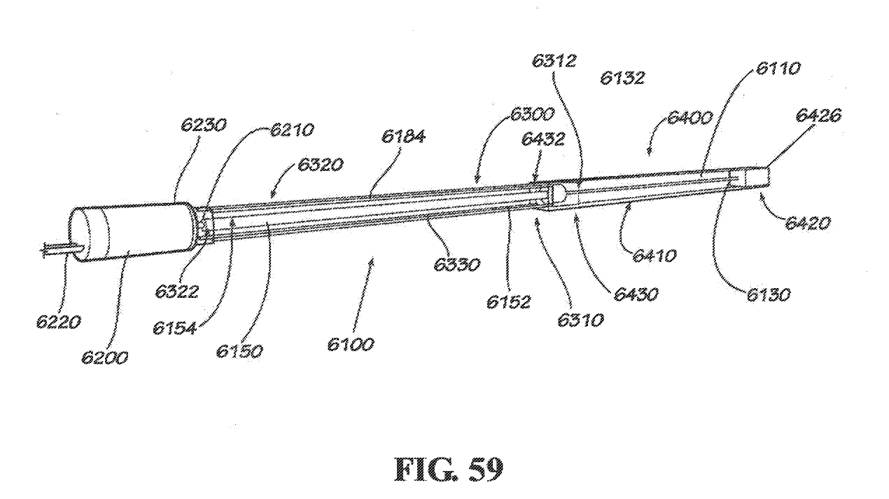

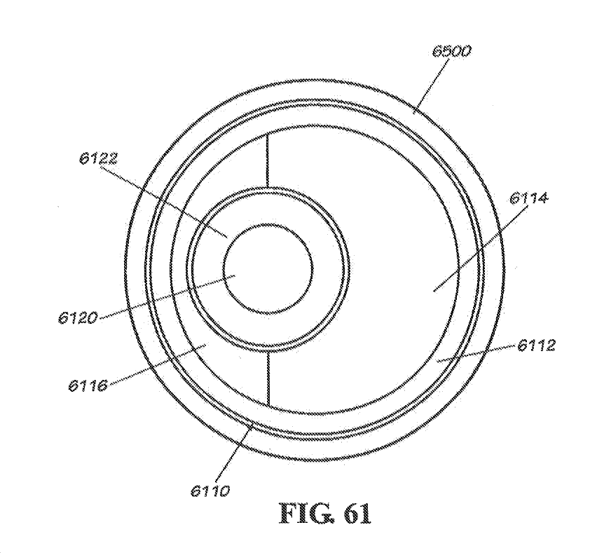

In an aspect, as illustrated in FIGS. 59-61 and 68, a catheter control cartridge 6100 can include a RSBT catheter 6110. In an aspect, the catheter RSBT 6110 can be constructed in the multiple fashions as described above. In an example, as illustrated in FIG. 61, the RSBT catheter 6110 is configured to be inserted into a needle 6500 found within the subject. The RSBT catheter 6110 can include an outer tube/catheter 6112. The outer tube 6112 can be comprised of numerous materials, including, but not limited to, stainless steel, nitinol, titanium, and various other plastics. However, nitinol is a preferred material for use in the outer tube 6112 because of nitinol is durable, can be effectively sterilized for reuse, provides some flexibility in an orthogonal direction, but is rotationally stiff.

In an aspect, as shown in FIG. 61, the outer tube 6112 of the RSBT catheter 6110 is configured to be smaller than the interior of the needle 6500, which leads to a small space 6111 between the two once the catheter 6110 is inserted into the needle 6500. This is done because the needle 6500 is inserted into a subject, it is normally done without the catheter 6110 inserted, which can lead to the needle 6500 being compressed or bent. By configuring the RSBT catheter 6110 to be smaller than the interior of the needle 6500, a little extra space is created between the outer wall 6112 of the catheter 6110 and the inner wall of the needle 6500 to allow the RSBT catheter 6110 to be smoothly inserted into the needle 6500, and subsequently rotated.

The outer tube/catheter 6112 of the RSBT catheter 6110 retains the shield 6114. The shield 6114 can have the properties and physical dimensions of the various shields discussed in the aspects above. The shield 6114 can have a specific radiation emission angle or opening, as discussed above. The shield 6114 can be made of a variety of materials that have properties that stop the penetration of radiation. The shield 6114 can be comprised of, but not limited to, osmium, gold, silver, uranium, tungsten, lead, bismuth or platinum. In an aspect, the shield 6114 is coupled to a window 6116. The window 6116 can have the properties and physical dimensions of the various windows discussed in the various aspects above. The window 6114 can be made of a variety of materials having a lower density than the material being used in the shield 6114, allowing for the penetration of radiation through the window 6116. In an aspect, the window 6116 is comprised of a plastic. In a preferred embodiment, the window 6116, and possibly the shield 6114, would be capable of being sterilized. The window 6116 is coupled to the shield 6114 to contain the radiation source 6120. The window 6116 and the shield 6114 can be coupled to one another in various ways, including, but not limited to, a tongue-groove combination, fasteners, adhesive, or the like. In an aspect, window 6116 can rest within a cutout portion of the shield 6114 and be retained within the cutout portion through the inner surface of the catheter tube 6112, with ends of the window 6116 abutting sides of the cutout portion of the shield.

The radiation source 6120 can comprise any radioactive material that can be used to deliver radiation as desired, including, but not limited to, the materials disclosed above. The radiation source 6120 can be contained within a radiation tube/catheter 6122. The radiation tube 6122 can have the properties and physical dimensions of the various radiation tubes/catheters (e.g., catheters 320 in FIGS. 42A & C-D) discussed in the various aspects above. The radiation tube 6122 can be comprised of a variety of different materials, including, but not limited to, nitinol, stainless steel, titanium, titanium alloy, and the like. In some aspects, the shield 6114 and window 6116 of the RSBT catheter 6110 are configured to have a space 6124 between the shield/window combination and the radiation tube 6122 when the radiation source 6120 is inserted. Such a configuration allows easier placement and removal of the radiation tube 6122 between the shield 6114 and window 6116, as well as allowing for separate manufacturing of the RSBT catheter 6110 and the radiation source 6120 (i.e., the RSBT catheter 6110 can be manufactured by a different manufacturer than the radiation source). In an aspect, the shield 6114, window 6116, and radiation source 6120 are located at a distal end 6130 of the catheter 6110.

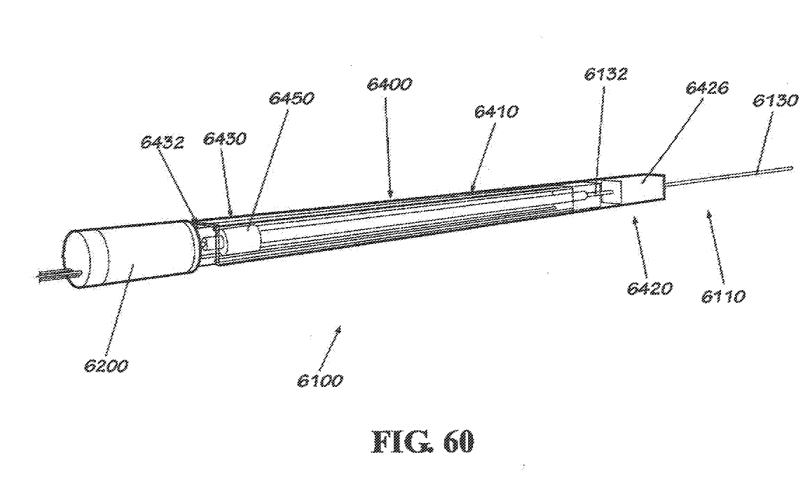

As shown in FIGS. 59-60, the RSBT catheter 6110 can be connected to a lead screw 6150, or other type of advancing mechanism, at a proximal end 6132 of the catheter. The lead screw 6150 can be made of a variety of materials, including, but not limited to aluminum, stainless steel, titanium, titanium alloy, plastic, and the like. In an aspect, the lead screw 6150 is made of a lightweight material that is not flexible. A light weight material can be desirable in order to reduce the overall weight of the RSBT catheter cartridge 6100.

The lead screw 6150 has a distal end 6152 and a proximal end 6154, with the proximal end 6132 of the RSBT catheter 6110 being coupled to the distal end 6152 of the lead screw 6150. In an aspect, RSBT catheter 6110 is configured to be removably coupled to the distal end 6152 of the lead screw 6150. In an aspect, the distal end 6152 of the lead screw 6150 can use a clamping device or other fastener means to removeably attach the RSBT catheter 6110. In other aspects, the RSBT catheter can be permanently attached to the distal end 6152 of the lead screw 6150. In an aspect, the lead screw 6150 can include an outer surface 6154 that can be threaded, discussed in more detail below.

In an aspect, the lead screw 6150 can be driven by a motor 6200. In an aspect, the motor 6200 is a stepper motor 6200 with a drive shaft 6210, a controller 6220, and a housing 6230. In an exemplary aspect, the stepper motor 6200 is a Faulhaber ADM 1220 stepper motor. However, other models of stepper motors from Faulhaber, as well as other manufacturers of stepper motors can be used to drive the lead screw 6150. The drive shaft 6210 is configured to rotate in a forward direction and a backward direction. The controller 6220 controls the activation of the motor 6200 and the rotational direction of the drive shaft 6210 as well.

The housing 6230, while protecting the inner components of the motor 6200, provides access to an exposed end of the drive shaft 6210. The exposed end of the drive shaft 6210 can protrude past the housing 6230, or the housing 6230 can provide an opening to the exposed end of the drive shaft 6210, such that the exposed end of the drive shaft 6210 can be coupled to the proximal end 6154 of the lead screw 6150. In an aspect, the proximal end 6154 of the lead screw 6150 can be removably coupled to the drive shaft 6210. Various fastening and connector means can be used to couple the proximal end 6154 of the lead screw 6150 to the drive shaft 6210. In an aspect, the fastening and connector means can include a plurality of slots found on the proximal end 6154 of the lead screw 6150 that correspond to a plurality of protrusions on the drive shaft 6210, making a male/female type connection. Clips, clamps, and the like can be used to lock the components in place.

In other aspects, the motor 6200 can include any motor capable of inserting or withdrawing the advancing mechanism 6150 into the shell 6400 in a controlled manner. In an aspect, the motor 6200 can include any motor capable of driving the lead screw 6150 in a rotational manner, pushing or pulling the lead screw 6150 into and out of the shell 6400 of the catheter cartridge 6100, discussed further below. In an aspect, a SQUIGGLE micro motor from New Scale Technologies can be used. However, it is preferred that a motor 6200 that provides precise control of the movement of the lead screw 6150 is used.

In other aspects, more than one motor 6200 can be used to drive the lead screw 6150. For example, in an aspect where it is desired that the path of the radiation emission does not rotate, a second motor can be configured to rotate the RSBT catheter 6110 to counteract the rotation to the lead screw 6150 caused by the first motor 6200.

In an aspect, as shown in FIG. 59-60, a carriage 6300 can be coupled to the housing 6230 of the motor 6200. The carriage 6300 can include a distal end 6310 and a proximal end 6320. The distal end 6310 and the proximal end 6320 of the carriage 6300 can be formed of open ended structures 6312, 6322. In an aspect, the proximal end 6320 of the carriage 6300 is connected to the housing 6230 of the motor 6200 approximate the access point of the drive shaft 6210. Various fastening means can be employed to connect the open ended structure 6322 of the proximal end 6320 of the carriage 6300 to the housing 6230 of the motor 6200. The open ended structure 6322 of the proximal end 6320 allows the lead screw 6150 to connect to the drive shaft 6210 of the motor 6200 while being housed within the carriage 6300, discussed in more detail below.

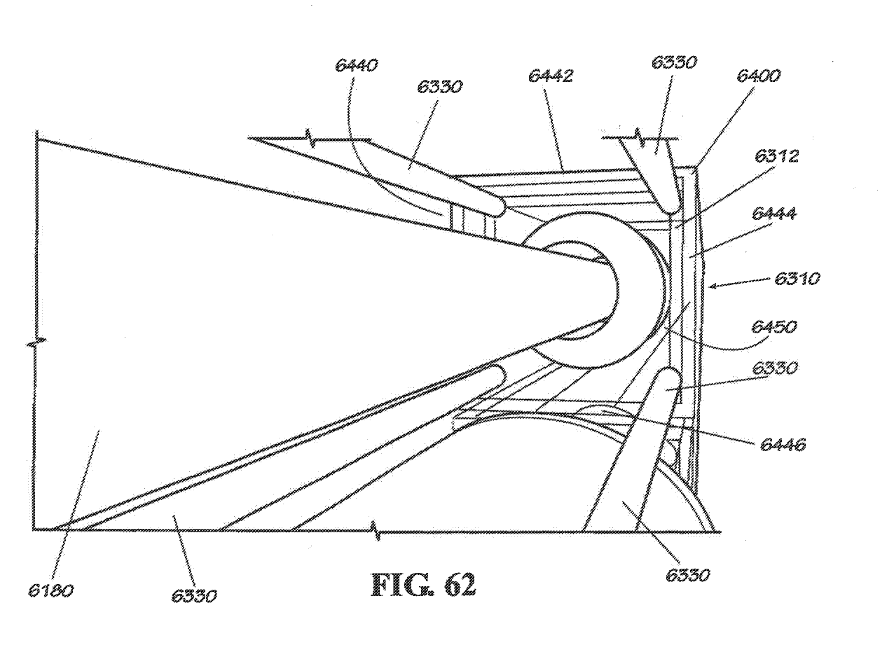

In an aspect the open ended structure 6312 of the distal end 6310 of the carriage 6300 can have a U-shaped cross section (shown in FIG. 62), allowing side access through the opened ended structure 6312. The distal and proximal ends 6310, 6320 can be connected by supporting structures 6330. In a further aspect, the carriage 6300 can be aligned to surround the lead screw 6150 in the same axial direction as the axial direction of the lead screw 6150 as it is connected to the drive shaft 6210 of the motor 6200.

As discussed in more detail below, the carriage 6300 is configured to engage with the interior of the shell 6400, and as such, the shape of the carriage 6300 can match the shape of the interior of the shell 6400 to promote the interaction between the two components 6300, 6400. In an aspect 6400, the shell 6400 has a cube shape along its length. In this aspect, as shown in FIGS. 59 and 62, the supporting structure 6330 can be comprised of 4 rods 6330 that are oriented in a parallel fashion relative to one another in order to match the shape of the interior of the shell to engage the interior of the shell 6400. The rods 6300 can be connected to the end structures 6312, 6322 of the carriage 6300. In this aspect, the rods 6300 are oriented to align within the corners of the interior of the shell 6400, preventing the carriage 6300 from rotating within the shell 6400.

In other aspects, the supporting structures 6330 can take various other forms. For example, the supporting structures 6330 can form a four full sided structure when connected. However, using rods 6330 oriented in the fashion discussed above cuts down on the overall weight of the carriage 6300 while providing stable support for the carriage 6300. In addition, by orienting the four rods 6330 in an equidistant manner, more space is provided between the lead screw 6150 and the boundaries of the carriage 6300. Given the size needs for the catheter control cartridges 6100 (i.e., multiple cartridges being used in a very small space), there is not much clearance room between the components, and therefore, any additional space can be valuable. Additionally, the use of rods 6330 allows access to the lead screw 6150 when needed. In an aspect, the carriage 6300 can be comprised of steel or other types of sturdy, durable material.

In an aspect, the carriage 6300 is configured to interact with a shell 6400, as illustrated in FIGS. 59-60, 62-64 and 67. The shell 6400 includes a hollow body 6410 with a distal end 6420 and a proximal end 6430. The ends 6420, 6430 can include openings 6422, 6432 to the interior of the hollow body 6410 of the shell 6400, with the opening 6432 of the proximal end 6430 configured to receive the carriage 6300 and the opening 6422 of the distal end 6420 configured to receive the catheter 6110. In an aspect, the distal end 6420 of the shell can include a cylinder extension 6424. The cylinder extension 6424 can be aligned with the opening 6422 of the distal end 6420, allowing the catheter 6110 to pass through its interior. In addition, the cylinder extension 6424 can be configured to engage and removably lock with components of a cartridge magazine 6600 discussed below.

The hollow body 6410 of the shell 6400, and the respective openings 6422, 6432, are configured to slidably and controllably receive the I-RSBT catheter 6100, the lead screw 6150, and the carriage 6300. In an aspect, the shape of the shell 6400 is configured to prevent the rotation of the carriage 6300 while the lead screw 6150 is being rotated by the motor 6200, discussed in detail below. In an example, as shown in FIGS. 59-60, 62-64, the shell 6400 has a rectangular shape that matches the shape of the carriage 6300. In this example, the rods 6330 of the carriage 6300 are oriented and aligned in a manner that the rods 6330, when engaging the interior of the shell 6400, are contained within the corners of the shell 6400. In other aspects, the shape of the shell 6400 can have other shapes that correspond with the shape and construction of the other components of the system 6000, including the carriage 6300 and lead screw 6150.

In an aspect, the shell 6400 can have four rectangular sides 6440, 6442, 6444, 6446, as shown in FIG. 62. In another aspect, one of the four rectangular sides 6446 can be removable in order to provide access into the interior of the shell 6400. By providing a removable side 6446, an individual can gain access to the interior of the shell 6400, as well as to components of the catheter control cartridge 6100 that are contained within the shell 6400 (e.g., the lead screw 6100, the carriage 6300, and the RSBT catheter 6110) when the system 6000 is not in use. The removable side 6446 can be secured to adjacent sides 6440, 6444 through removable set screws, clips, clamps, and other fastening means.

In an aspect, the shell 6400 can be comprised of various materials, including, but not limited to, carbon fiber, aluminum, stainless steel, sheet metal, and the like. In an aspect, while a variety of materials can be used, it is preferred that the shell material 6400 have a thickness of 0.5 mm or less. The distal end 6420 of the shell 6400 can include a radiation shield 6426. The radiation shield 6426 can be made of any material that is suitable to substantially block any radiation coming from the radiation source 6120 when the catheter 6100 is retained within the shell 6400 while not actively being used. For example, the material of the radiation shield 6426 can include, but is not limited to, stainless steel, brass, lead and the like.

The proximal end 6430 of the shell 6400 can include an advancing mechanism receiver 6450. The advancing mechanism receiver 6450 can be secured within or at the proximal end 6430 of the shell 6400. In an aspect, the advancing mechanism receiver 6450 can include a screw nut 6450. The screw nut 6450 can be utilized when the advancing mechanism 6150 is a lead screw 6150 as described above. The screw nut 6450 can secured within the proximal end 6430 approximate the opening 6432. The screw nut 6450 can include a threaded interior surface (not shown) that corresponds to the threaded exterior surface of the lead screw 6150. The screw nut 6450 is secured in a fashion to prevent any the rotation of the screw nut 6450 within the shell 6400 when interacting with threaded surface 6154 of the lead screw 6150. In an aspect, set screws can be used to secure the screw nut 6450 to the proximal end 6430 of the shell 6400. In other aspects, various other fasteners and securing means can be used to secure the screw nut 6450. In an aspect, the screw nut 6450 can be a part of a flange closing off the proximal end 6430 of the shell 6400 that only provides access through the opening of the screw nut 6540. In this aspect, the flange can include apertures that engage the support structures of the carriage 6300, preventing the carriage 6300 from rotating within the shell 6400. In other aspects, the advancing mechanism receiver 6450 can include any other receiving device or mechanism that is configured to receive the advancing mechanism 6150. In some of these aspects, the advancing mechanism receiver 6450 can be further configured to assist in controlling the movement of the advancing mechanism 6150 within the shell 6400.

In an exemplary aspect, as shown in FIGS. 59-60, the catheter control cartridge 6100 is set up to operate in the following fashion. The I-RSBT catheter 6110 is secured to the distal end 6152 of the lead screw 6150. The distal end 6310 of the carriage 6300 is received within the proximal end 6430 of the shell 6400 and the distal end 6152 of the lead screw 6150 is threaded into the screw nut 6450 of the shell 6400. When the motor 6200 is activated in the forward direction, the drive shaft 6210 rotates the lead screw 6150. The threated surface 6154 of the lead screw 6150 and the screw nut 6450 pull the lead screw 6150, the carriage 6300, and the catheter 6110 further into the shell 6400, as shown in FIG. 60. The carriage 6300 is configured to engage the inner surfaces of the shell 6400, preventing the motor 6200 from spinning in place, but advancing the lead screw 6150. As the lead screw 6150 advances, the catheter 6110 rotates within the shell 6400 and out through the distal end 6430, as shown in FIG. 60.

In an aspect, as illustrated in FIGS. 58, 63-64, 66 and 68, the I-RSBT catheter delivery system 6000 can utilize a cartridge magazine 6600 to contain and align multiple catheter control cartridges 6100 and their I-RSBT catheters 6110 for simultaneous use. The magazine 6600 includes a distal end 6610 and a proximal end 6620. When placed within the magazine 6600, the catheter control cartridges 6100 are arranged such that the distal end 6110 of the I-RSBT catheter 6110 is aligned with the distal end 6610 of the magazine 6600, and the motor 6200 is oriented approximate the proximal end 6620 of the magazine 6600. The magazine 6600 is further configured to allow the multiple catheter control cartridges 6100 to be removed and realigned accordingly.

In an aspect, the magazine 6600 contains multiple shelves 6630 within its interior. The shelves 6630 support the catheter control cartridges 6100 when placed within the magazine 6600. In an aspect, the multiple shelves 6630 are independently adjustable within the interior of the magazine 6600. In an aspect, the interior of the side walls 6632 of the magazine 6600 include adjustable shelf retaining components that allow the height of each individual shelf 6630 to be adjusted independently.

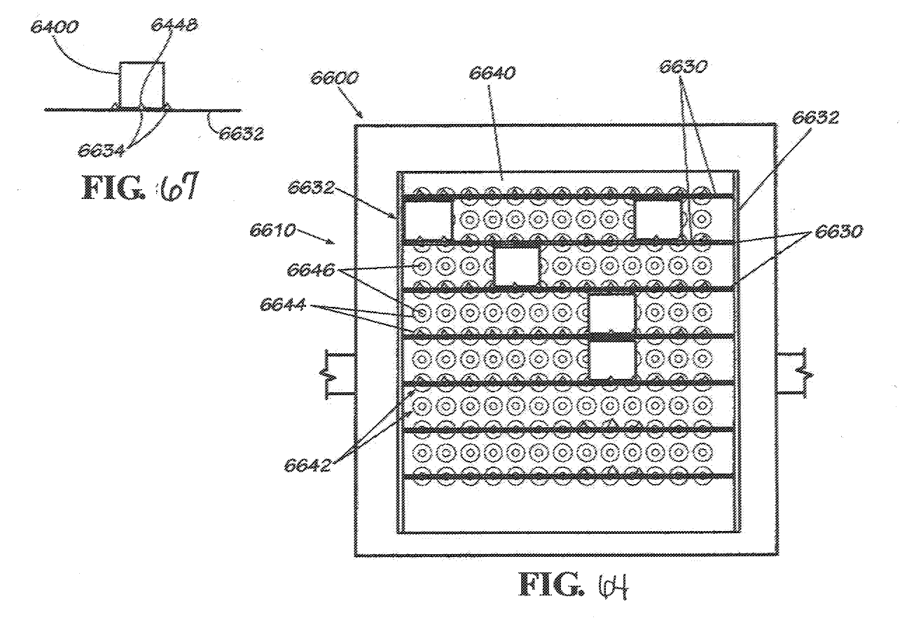

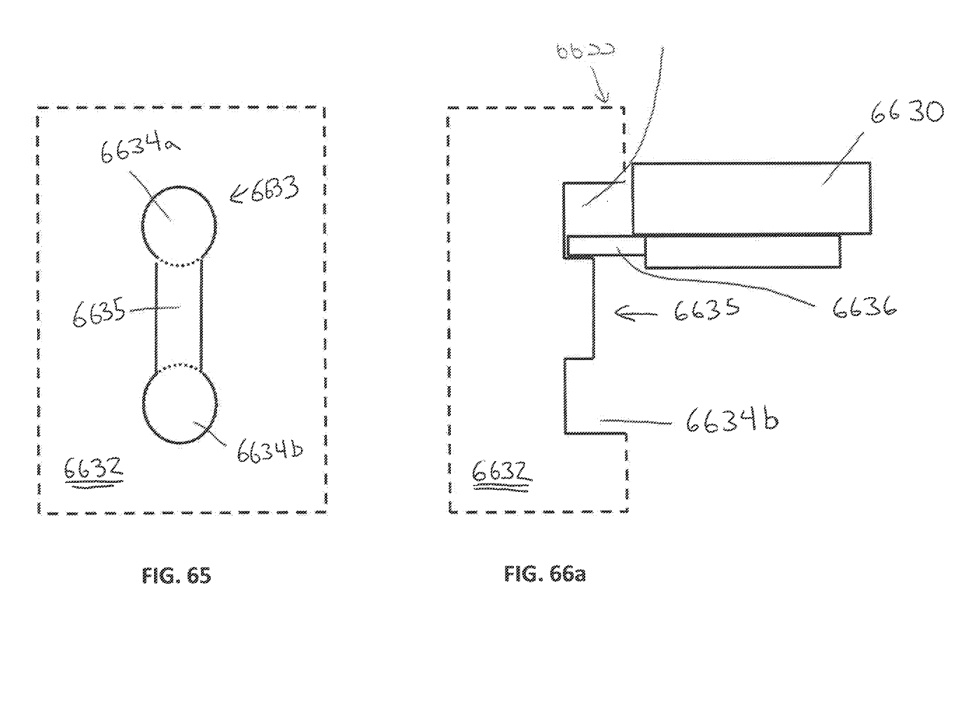

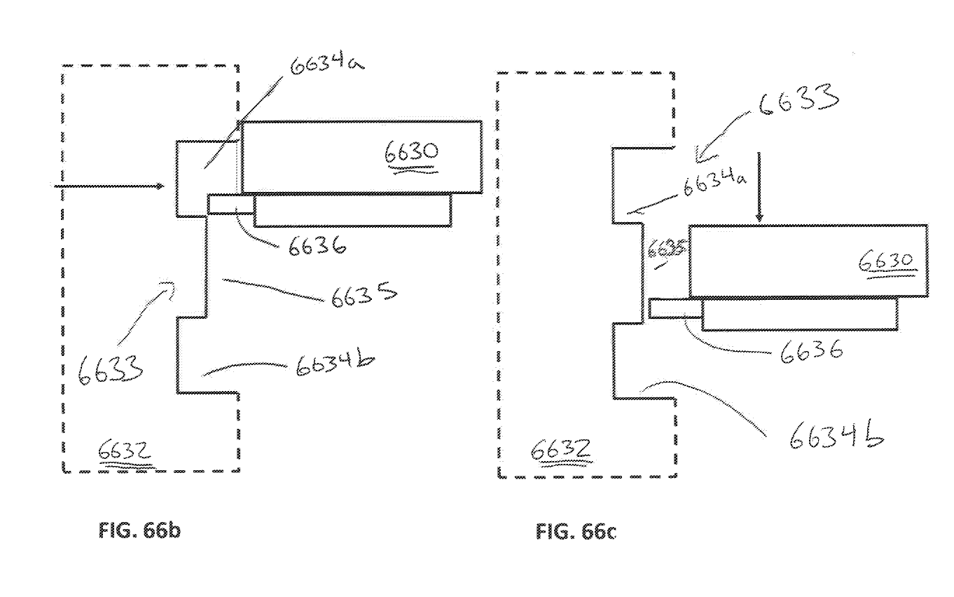

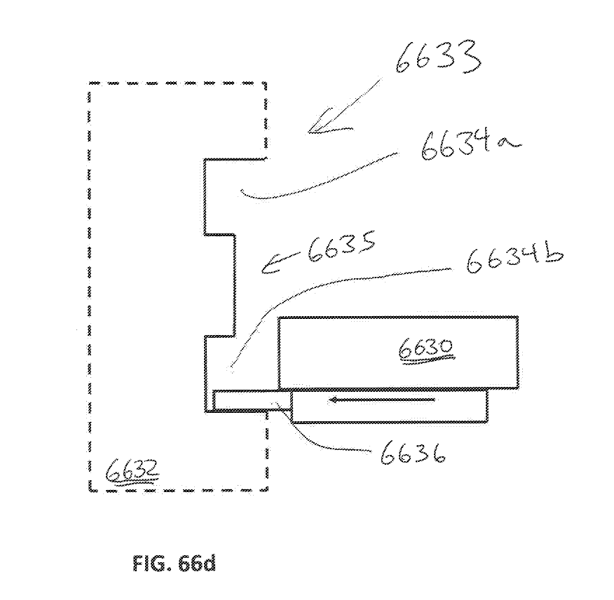

In an exemplary aspect, illustrated in FIGS. 65-66a-d, the adjustable shelf retaining components can include multiple mounts 6633 along the sides of the interior walls 6632 of the magazine 6600. The mounts 6633 should be aligned within the same plane for each shelf 6630. The mounts 6633 include bores 6634a, 6634b vertically aligned with one another that are connected by a slot 6635. The bores 6634 can have a depth greater than the depth of the connecting slot 6635. The shelves 6632 include pegs 6636 on their sides that are configured to fit within the bores 6634, as well as travel within the slot 6635. The number of pegs 6636 corresponds to the number of mounts 6633 for each shelf 6632. For example, each shelf 6630 can include two pegs 6636 on each side of the shelf 6630, with two mounts 6633 on each interior wall 6632 for each shelf 6630. The pegs 6636 can be spring loaded.

To adjust the height of the shelves 6632, the springs of the pegs 6636 are compressed (FIG. 66a-b), allowing the peg 6636 to travel within the slot 6635 (FIG. 66c) to another bore 6634b. When the peg 6636 reaches the bore 6634b, the spring releases and keeps the peg 6636 from exiting the bore 6634 into the slot 6635 (FIG. 66d). As illustrated, the mounts 6633 have two bores 6634, allowing the shelves 6632 to be adjusted between two different heights within the magazine 6600. The mounts 6633, through the positioning of the two bores 6634, allow individual shelves 6630 to be positioned at two different heights within the magazine 6600. In an exemplary aspect, the bores 6635 of each mount 6633 are separated by 5 mm, and the mounts 6633 are spaced apart vertically from one by 5 mm, making the default height between the shelves 6630 10 mm, and allowing each shelf 6630 to be adjusted 5 mm.

In another aspect, the mounts 6633 can be oriented in a different fashion along the inner walls 6632. In an example, the slots 6635 of mounts on one inner wall 6632 can be oriented at an angle between the bores 6634a-b. In an exemplary example, the slot 6635 can be oriented at 450. The peg 6636 can be slid into the top bore 6634a, slide down the slot 6635 to the bottom bore 6634b, effectively keeping it in place. In such aspects, the pegs 6636 assigned for use with such mounts 6633 do not need to be spring loaded. With such an approach, a user can move and unlock the shelf 6630 only using two spring loaded pegs 6636, with the other non-spring loaded pegs 6636 resting in the bore 6634b, confined there by the off angled slot 6635. A user could then raise or lower the entire shelf by unlocking the two spring loaded pegs 6636, rather than four.

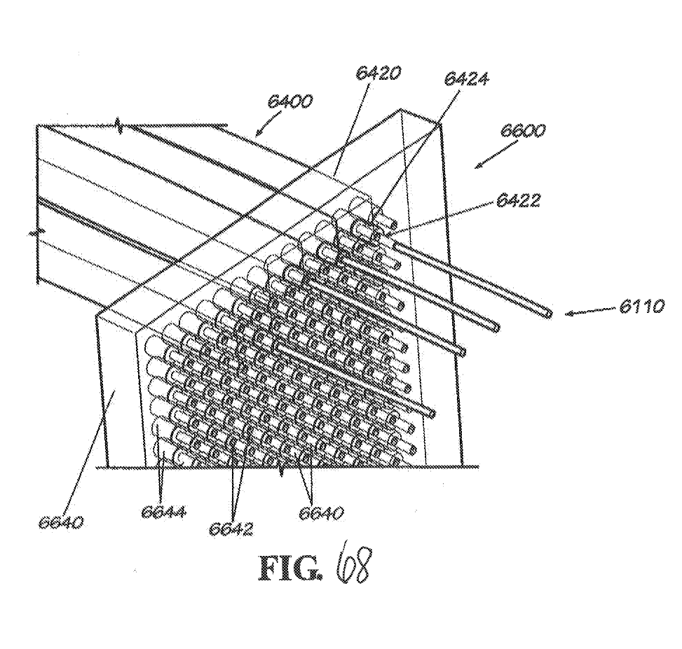

In an aspect, as shown in FIG. 67, the shelves 6630 can include cartridge securing means. In an aspect, the cartridge securing means are configured to prevent the cartridges 6100 from shifting from side to side on the shelves 6630. In another aspect, the cartridge securing means can assist in aligning the cartridges 6100, and more specifically the catheters 6110, with openings provided in a cartridge template 6640, discussed below. In an aspect, the cartridge securing means can include a protrusion and groove combination that the cartridges engage. In this aspect, the exterior bottom side of the shell 6400 of the cartridge 6100 can include a groove that receives a protrusion found on the shelf 6630. In an example of this aspect, the grooves found of the shelves 6630 can be uniformly distributed across the width of the shelf. In other aspects, other securing mechanisms can be used.

A cartridge template 6640 can be found at the distal end 6610 of the magazine 6600, as shown in FIGS. 64 and 68. The cartridge template 6640 can include numerous openings 6642. In an aspect, the openings 6632 are distributed uniformly across the template 6640. In an exemplary aspect, the openings 6632 are distributed in a 10 mm×10 mm grid. The openings 6642 of the template 6630 are configured to receive the I-RSBT catheters 6110 and allow them to transverse the openings 6642 in an axial direction of the catheter 6110. In an aspect, the openings 6642 can comprise a two tier opening 6642, with the first tier 6644 configured to engage cylinder extension 6424 of the shell 6400 and the second tier 6646 configured to allow the catheter 6110 to transverse the opening 6644 in the axial direction. Different securing means can be used to secure the magazine 6600 to prevent moving when it is in use. In an aspect, fasteners, clamps, vises, and the like can be used to secure the magazine 6600 in place.

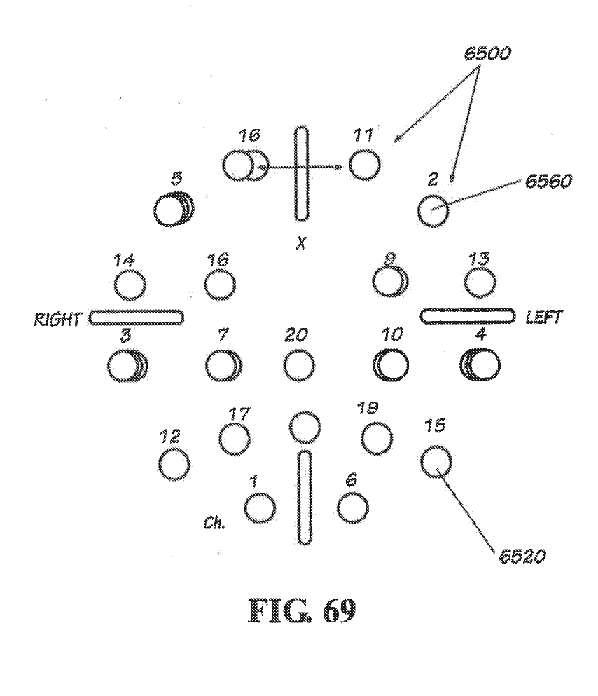

In an aspect, the I-RSBT catheter 6110 of catheter control cartridge 6100 is configured to engage with a needle 6500, shown in FIGS. 69-71. The needles 6500 can include, but are not limited to, interstitial needles and intracavitary applicators. The needle 6500 has a distal end 6510 and a proximal end 6520 that both provide access into a tubular body 6530 of the needle 6500. The dimensions of the needle 6500 can vary based upon the particular application. In an aspect, the diameter of the tubular body 6530 is of sufficient size to receive the I-RSBT catheter 6110 with a small space between the outer surface of the catheter 6110 and the inner surface of the tubular body 6530 of the needle 6500. The needle 6500 can be comprised of a number of different materials. In an exemplary aspect, the needle 6500 can be made of nitinol, given that such material is ultrasound imaging, CT and MRI compatible, as well as sterilizable. Other materials, including, but not limited to, plastic, titanium, and stainless steel can be used.

The needle 6500 can be placed within the subject under ultrasound guidance, other inter-body guidance techniques, or other known guidance methods. The distal end 6510 of the needle 6500 is placed within the body of the subject, with the proximal end 6520 exposed outside the subject, providing access for the I-RSBT catheter 6110 to enter the subject. The alignment and placement of the needles 6500 is dependent on the location of the tumor being treated, and where the user believes the I-RSBT needs to be delivered. In an aspect, FIG. 68-70 show the needed position of the needles 6500 in a subject, with the proximal ends 6520 exposed outside of the body of the subject. As shown, twenty needles 6500 are being used to treat the tumor, with each needle 6500 can be assigned an identifying number (as shown, numbers 1-20). Based upon the components of the I-RSBT delivery system 6000, the catheters 6110 can be aligned with the magazine 6600 to apply the I-RSBT at the locations identified above.

In an aspect, the spacing of the needles 6500 within the subject, along with the cross sectional size of the catheter control cartridges as set up within the magazine 6600, can dictate the number of catheter control cartridges 6100, and cartridges 6100 alignment, that can be used at a single time. For example, if the cross sectional area of the cartridges 6100 is 9.5 mm×9.5 mm (with the catheter 6110 being oriented within the center of the shell 6400), and any two needles 6500 are spaced closer than 9.5 mm to one another, the I-RSBT will need to be delivered at different times for those two needles 6500.

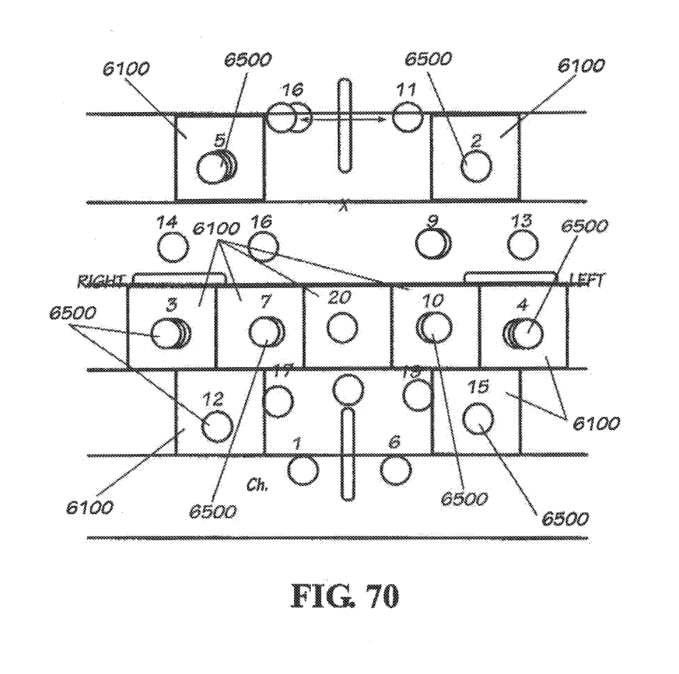

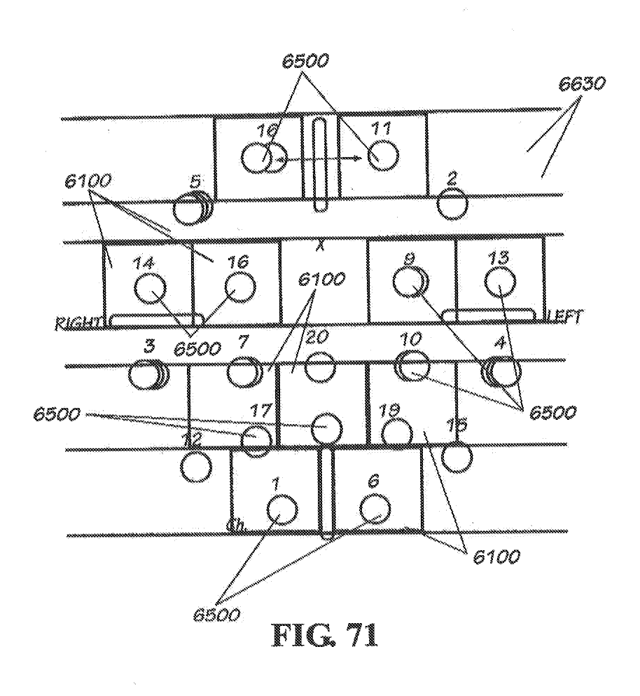

In an aspect, FIGS. 70-71 illustrate a case of 1-RSBT application in which I-RSBT is applied to the subject at two different instances. As shown in FIGS. 70-71, the needles 6500 can be positioned in different planes and at different spacing from one another. In round 1 (shown in FIG. 70), nine cartridges 6100 are aligned with nine needles 6500 to apply the I-RSBT application. As shown, the nine cartridges 6100 are positioned at various positions of the shelves 6630. FIG. 71 shows round 2 of the application of I-RSBT. In this round, ten cartridges 6100 are used with ten needles 6500 to apply the I-RSBT. As shown, the cartridges 6100 are aligned in different positions on the shelves 6630. In addition, the shelves 6630 have been aligned differently (i.e., adjustment of height of the shelves 6630 within the magazine 6600) in comparison to the shelves 6630 as shown in FIG. 70 (round 1). The height of the shelves 6300 can be adjusted in order for the cartridges 6100 to be aligned with the ten needles 6500 that were unused in round 1. In an aspect as shown in FIG. 70, the needles 6500 may not be aligned directly with the center of the cartridges 6100. In such instances, using catheters 6100 made of flexible material (e.g., nitinol) can allow the cartridges 6100 to be used.

In an aspect, the I-RSBT delivery system 6000 can employ a controller 6700 to control the application of I-RSBT. In an aspect, the controller 6700 can be configured to connect and interface with the motors 6200 of each control cartridge 6100 that is being used. In an aspect, the controller 6700 can include a driver capable of controlling each motor being used. In other aspects, the controller 6700 can include a computer 2400 as described above. Various applications associated with the computers 2400, 5101 can control the motors 6200 of the control cartridges 6100 to apply the needed doses of I-RSBT.

In an aspect, when the motors 6200 of the cartridges 6100 are activated, the drive shaft 6210 will begin to rotate, rotating the lead screw 6150. As the lead screw 6150 rotates, the threaded surface of the lead screw 6150 engages the threaded surface of the screw nut 6450. Since the cartridge 6100, by way of the shell 6400, is secured on the shelf 6630 through the securing means, the lead screw 6150, catheter 6110 and carriage 6300 advance towards the distal end 6410 of the shell 6400 and the template 6640. The catheters 6110 can then engage the openings 6642 of the template 6640, and exit into the needles 6500 at the proximal ends 6520. As the lead screw 6150 continues to rotate, the catheter 6110 rotates as well, delivering the radiation in a helical pattern. The controller 6700 can control the activation of the motor 6200 until the catheter 6100 reaches the appropriate depth and the window 6116 and shield 6614 of the catheter 6100 is positioned in the correct direction to apply the radiation to the desire location within the subject. The controller 6700 can control this operation automatically, through the use of various applications, or can be controlled manually to stop the motors 6200 based upon the user providing input based upon observations (e.g., ultrasound guidance, CT or MRI guidance).

While the systems, devices, apparatuses, protocols, processes, and methods have been described in connection with exemplary embodiments and specific illustrations, it is not intended that the scope be limited to the particular embodiments set forth, as the embodiments herein are intended in all respects to be illustrative rather than restrictive.

To the extent necessary to understand or complete the disclosure of the present invention, all publications, patents, and patent applications mentioned herein are expressly incorporated by reference to the same extent as though each were individually so incorporated.

Unless otherwise expressly stated, it is in no way intended that any protocol, procedure, process, or method set forth herein be construed as requiring that its acts or steps be performed in a specific order. Accordingly, in the subject specification, where description of a process or method does not actually recite an order to be followed by its acts or steps or it is not otherwise specifically recited in the claims or descriptions of the subject disclosure that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification or annexed drawings, or the like.

It will be apparent to those skilled in the art that various modifications and variations can be made in the subject disclosure without departing from the scope or spirit of the subject disclosure. Other embodiments of the subject disclosure will be apparent to those skilled in the art from consideration of the specification and practice of the subject disclosure as disclosed herein. It is intended that the specification and examples be considered as non-limiting illustrations only, with a true scope and spirit of the subject disclosure being indicated by the following claims.

What is claimed is:

1. A method for rotating shield brachytherapy (RSBT), comprising:
selecting a plurality of radiation shields for RSBT, each one of the plurality of radiation shields having a specific radiation emission angle; and
applying a treatment plan by delivering radiation over a predetermined period through a specific sequence of the plurality of the plurality of radiation shields.

2. The method of claim 1, wherein the selecting action comprises determining the patient-specific sequence of radiation shields, and wherein the patient-specific sequence comprises an ordered combination of the plurality of radiation shields.

3. The method of claim 1, wherein the selecting action comprises selecting at least one radiation shield of the plurality of radiation shields based at least on one or more of a plurality of radiation source positions, shape of a volume of tissue to be treated, or a predetermined radiation dosage.

4. The method of claim 1, wherein each one of the radiation shields has a specific radiation emission angle.

5. The method of claim 1, wherein each one of the radiation shields is formed from a high-atomic-number compound, and wherein the high-atomic-number compound comprises one of tungsten, lead, silver, gold, platinum, or bismuth.

6. A medical device, comprising:
a shaft formed from at least two materials, wherein a first material of the at least two materials permits transmission of a first amount of radiation, and a second material of the at least two materials permits transmission of a second amount of radiation; and
a coating that covers at least a portion of the shaft, the coating configured to reduce toxicity of the shaft when inserted into a subject submitted to interstitial brachytherapy.

7. The medical device of claim 6, wherein the first material and the second material have a common interface defining a plane that contains a longitudinal axis of the shaft.

8. The medical device of claim 7, wherein the first material defines a first azimuthal angle at a cross-sectional plane of the shaft, and wherein the second material defines a second azimuthal angle at the cross-sectional plane, the sum of the first azimuthal angle and the second azimuthal angle is approximately 360 degrees.

9. The medical device of claim 6, wherein the first material defines a first azimuthal angle at a cross-sectional plane of the shaft, the first azimuthal angle is greater than zero and less than approximately 360 degrees.

10. The medical device of claim 6, wherein the second material defines a second azimuthal angle at a cross-sectional plane of the shaft, the second azimuthal angle is greater than zero and less than approximately 360 degrees.

11. The medical device of claim 6, wherein the shaft has a circular cross-section with an on-center lumen having a circular cross-section.

12. The medical device of claim 6, wherein the shaft has a circular cross-section with an off-center lumen having a circular cross-section.

13. The medical device of claim 6, wherein the first material is a first high-density material, and wherein the second material is a second high-density material.

14. The medical device of claim 13, wherein the control unit comprises a grasping mechanism.

15. The medical device of claim 13, further comprising a control unit configured to couple to the docking device, the control unit is configured to rotate the shaft according to a predetermined treatment plan.

16. The medical device of claim 6, further comprising a docking device attached to the shaft.

17. An apparatus for providing therapeutic radiation, the apparatus comprising:
a capsule;
a radiation source within the capsule; and
a wire attached to the capsule.

18. The apparatus of claim 17, wherein the radiation source is Gadolinium-153.

19. The apparatus of claim 18, wherein the radiation source has a specific activity within the range of 151 Ci/g and 250 Ci/g.

20. The apparatus of claim 18, wherein the radiation source has a specific activity greater than 150 Ci/g.

21. The apparatus of claim 17, wherein the radiation source is configured to provide a high-dose-rate of radiation to a target, the high-dose-rate of radiation comprises radiation of at least 100 keV.

22. The apparatus of claim 17, further comprising an applicator tube configured to guide the capsule to a target.

23. The apparatus of claim 22, further comprising an afterloader system, the after loader configured to advance the capsule through the applicator tube by moving the wire.

24. The apparatus of claim 17, wherein the applicator tube has a maximum thickness of less than 0.33 millimeters.

25. A method of forming a therapeutic radiation capsule, the method comprising:
enclosing a radiation source in a capsule; and
attaching the capsule to a wire, the wire configured to guide the capsule to a target through an applicator tube.

26. The method of claim 25, wherein the radiation source is Gadolinium-153.

27. The method of claim 25, wherein the radiation source has a specific activity within the range of 151 Ci/g and 250 Ci/g.

28. The method of claim 25, wherein the radiation source is configured to provide a high-dose-rate of radiation to a target, the high-dose-rate of radiation comprises radiation of at least 100 keV.

29. A method of providing therapeutic radiation, the method comprising:
identifying a target;
providing an applicator tube configured to guide a capsule proximate to the target, the capsule enclosing a radiation source and having a wire attached to the capsule; and
guiding the capsule through the applicator tube proximate to the target by use of the wire.

30. The method of claim 29, wherein the radiation source comprises multiple gadolinium-153 wires each with a rotating shielded catheter.

31. The method of claim 29, further comprising releasing the radiation source from the capsule.

32. The method of claim 29, wherein the radiation source is Gadolinium-153.

33. The method of claim 32, wherein the radiation source has a specific activity greater than 150 Ci/g.

34. The method of claim 32, wherein the radiation source is configured to provide a high-dose-rate of radiation to a target, the high-dose-rate of radiation comprises radiation of at least 100 keV.

35. The method of claim 29, wherein a maximum diameter of the capsule is less than 1 mm.

36. The method of claim 29, wherein the wire is connected to an afterloader system, the after loader system configured to advance the capsule through the applicator tube by moving the wire.

37. A method for selecting an emission angle for use in single rotating-shield brachytherapy, the method comprising:
calculating a dose;
optimizing the calculated dose;
generating a first treatment plan based on the optimized dose;
generating a second treatment plan; and
selecting one of the first treatment plan or the second treatment plan.

38. The method of claim 37, further comprising receiving data indicative of a radiation treatment and a topology of a region to be treated.

39. The method of claim 37, further comprising receiving input parameters for azimuthal emission angle and an azimuthal emission step ratio for at least one anchor plan.

40. The method of claim 37, wherein the at least one anchor plan comprises three anchor plans.

41. The method of claim 37, further comprising generating output metrics.

42. The method of claim 41, wherein generating output metrics comprises generating at least one Pareto plot.

43. The method of claim 37, wherein optimizing the calculated dose comprises performing at least one of surface optimization and dose-volume optimization.

44. The method of claim 43, wherein dose-volume optimization comprises performing one or more simulated annealing calculations.

45. The method of claim 44, wherein performing one or more simulated annealing calculations comprises calculating one or more initial solutions by a surface optimization calculation, the surface optimization calculation optimizing a dose homogeneity on a high risk clinical target volume (HR-CTV) by a gradient-based least squares calculation.

46. A catheter delivery system configured to deliver I-RSBT in numerous locations, the system comprising:
a. at least one catheter control cartridge comprising:
i. a catheter comprising a radiation source;
ii. an advancing mechanism coupled to the catheter; and
iii. at least one motor configured to drive the advancing mechanism; and
b. a cartridge magazine holder configured to contain the at least one catheter control cartridge, the cartridge magazine holder comprising:
i. a template comprising at least one opening to receive the catheter of the at least one catheter control cartridge; and
ii. at least one shelf to support the at least one catheter control cartridge.

47. The catheter delivery system of claim 46, wherein the catheter further comprises an outer tube and a shield having a specific radiation emission angle, wherein the shield is configured to be retained within the catheter between the radiation source and the catheter.

48. The catheter delivery system of claim 47, wherein the catheter further comprises a radiation catheter configured to retain the radiation source, wherein the shield is placed between the outer tube and the radiation catheter.

49. The catheter delivery system of claim 48, wherein the radiation source is Gadolinium-153.

50. The catheter delivery system of claim 48, wherein the radiation source has a specific activity within the range of 151 Ci/g and 250 Ci/g.

51. The catheter delivery system of claim 47, wherein the advancing mechanism is configured to rotate the catheter to deliver radiation in a helical pattern.

52. The catheter delivery system of claim 47, further comprising at least one needle comprising an interior, an opening, and a hollow interior, wherein the at least one needle is configured to be placed within a body of a subject at a location and receive the catheter.

53. The catheter delivery system of claim 46, wherein the at least one catheter control cartridge further comprises a carriage comprising a proximal end and a distal end, the proximal end connected to the motor, and a shell comprising a hollow body, a proximal end with a proximal opening configured to receive the carriage and the catheter, and a distal end with a distal opening configured to receive the catheter, wherein the motor is further configured to drive the advancing mechanism and the catheter into the shell through the proximal opening and the catheter through the distal opening.

54. The catheter delivery system of claim 53, wherein the shell further comprises an advancing mechanism receiver secured approximate the distal end of the shell, wherein the advancing mechanism receiver is configured to assist in controlling the movement of the advancing member within the shell.

55. The catheter delivery system of claim 54, wherein the advancing mechanism further comprises a lead screw comprising a distal end and a proximal end, wherein the advancing mechanism receiver further comprises a screw nut configured to interact with the lead screw, wherein the catheter further comprises a distal end and a proximal end, wherein the proximal end of the catheter is removably coupled to the distal end of the lead screw, wherein the motor comprises a drive shaft configured to be coupled to the proximal end of the lead screw and to rotate the lead screw, wherein the carriage is configured to interact with the interior of the shell to prevent the shell from rotating when the drive shaft is driving the lead screw.

56. The catheter delivery system of claim 53, wherein the distal end of the shell further comprises a radiation shield.

57. The catheter delivery system of claim 53, wherein the distal end of the shell further comprises a cylinder extension aligned with the distal opening, wherein the at least one opening of the template further comprises a two tier opening, wherein the first tier is configured to receive the cylinder extension of the shell to secure the at least one catheter control cartridge in place within the cartridge magazine on the at least one shelf.

58. The catheter delivery system of claim 46, wherein the at least one catheter control cartridge further comprises a shell configured to receive the catheter, the shell comprising a removable side allowing access to the catheter when the catheter is within the shell.

59. The catheter delivery system of claim 46, wherein the at least one catheter control cartridge further comprises a shell configured to receive the catheter, wherein the shell comprises cartridge securing means configured to secure the at least one catheter control cartridge to the at least one shelf.

60. The catheter delivery system of claim 46, wherein the cartridge magazine further comprises inner walls and an adjustable shelf retaining component configured to allow the height of the at least one shelf to be adjustable along the inner walls of the cartridge magazine.

61. The catheter delivery system of claim 60, wherein the at least one shelf comprises a plurality of shelves, wherein at least a portion of the plurality of shelves is independently controllable.

62. An I-RSBT catheter delivery system configured to deliver I-RSBT in numerous precise locations over a small volume of a subject, the system comprising:
a. a plurality of needles, wherein at least one of the plurality of needles comprises a proximal end, a distal end, a hollow interior, and a proximal opening at the proximal end that extends into the hollow interior, wherein a portion of the needle is configured to be placed at one of the numerous precise locations of the subject;
b. a plurality of catheter control cartridges, at least a portion of the catheter control cartridges comprising:
i. a RSBT catheter configured to be inserted into the hollow interior of the needle, the RSBT catheter comprising;
A. a distal end;
B. a proximal end;
C. an outer tube configured to be smaller than the interior of the needle comprising a distal end and a proximal end;
D. a shield having an opening and comprised of a material of a first density;
E. a window secured approximate to the opening and comprised a material with a second density, wherein the first density is greater than the second density; and
F. a radiation source, wherein the shield and the window are positioned between the radiation source and the outer tube;
ii. a lead screw comprising;
A. a distal end, wherein the proximal end of the RSBT catheter is removably coupled to the distal end of the lead screw;
B. a proximal end; and
C. a threaded surface;
iii. a motor comprising:
A. a drive shaft configured for bi-directional rotation, wherein the proximal end of the lead screw is removably coupled to the drive shaft to be driven rotationally by the drive shaft;
B. a housing; and
C. a controller, wherein the controller controls the drive shaft;
iv. a carriage comprising:
A. a proximal end coupled to the housing of the motor,
B. supporting structures configured to provide access to the lead screw and the RSBT catheter; and
C. a distal end;
v. a shell configured to receive the carriage and the RSBT catheter, the shell comprising:
A. a hollow body comprising at least one removable side, wherein the hollow body is configured to match the shape of the carriage;
B. a proximal end having a proximal opening configured to receive the catheter and the carriage;
C. a distal end having a distal opening configured to receive the catheter; and
D. a nut screw secured approximate to the proximal opening and configured to pull or push the lead screw as the lead screw is rotated and move the catheter forward or backwards into and out of the shell, wherein the shape of the hollow body prevents the carriage from rotating within the hollow body of the shell;
c. a cartridge magazine holder comprising:
i. a distal end;
ii. a template located at the distal end, the template comprising:
A. a plurality of openings configured to receive the catheter of the at least a portion of the plurality of catheter control cartridges, wherein the plurality of openings are uniformly distributed across the template;
iii. a proximal end;
iv. side walls;
v. a plurality of shelves; and
vi. and a height adjustment mechanism to independently adjust the height of one of the plurality of shelves; and
d. a system controller configured to control the controller of the motors of the at least a portion of the plurality of catheter control cartridges, wherein the system controller can control the movement of multiple catheters of the at least a portion of the plurality of catheter control cartridges at the same time such that the catheter is positioned within the needle at a desired depth and a desired angle within the body of the subject.
